(19) United States
(12) Patent Application Publication
(10) Pub. No.: US 2023/0398125 A1
(43) Pub. Date: Dec. 14, 2023

(54) TREATING HEPATITIS

(71) Applicant: Albireo AB, Göteborg (SE)

(72) Inventors: Erik Lindström, Göteborg (SE); Britta Bonn, Göteborg (SE); Jan Mattsson, Kullavik (SE)

(21) Appl. No.: 18/207,996

(22) Filed: Jun. 9, 2023

Related U.S. Application Data
(60) Provisional application No. 63/350,693, filed on Jun. 9, 2022.

(51) Int. Cl. A61K 31/554; A61P 31/22; A61K 31/675

(52) U.S. Cl. CPC A61K 31/554; A61P 31/22; A61K 31/675

(57) ABSTRACT

Provided herein are methods for treating hepatitis B and/or D with an Na+/taurocholate co-transporting polypeptide (NTCP) inhibitor such as a compound of formula (I), or a pharmaceutically acceptable salt thereof, or a compound of formula (II), or a pharmaceutically acceptable salt thereof. Such methods can include decreasing the concentration of hepatitis B DNA, decreasing the concentration of hepatitis D DNA, decreasing hepatitis B surface antigen, and decreasing hepatitis B core antigen (HBcAg).

### CROSS-REFERENCE TO RELATED APPLICATIONS

This application claims priority to U.S. Provisional Application No. 63/350,693 filed on Jun. 9, 2022, which is incorporated herein by reference in its entirety.

### TECHNICAL FIELD

The present disclosure is related to methods for treating hepatitis B and/or D with one or more NTCP inhibitors such as 1,5-benzothiazepine and 1,2,5-benzothiadiazepine derivatives, or pharmaceutically acceptable salts thereof. The present disclosure also relates to methods for decreasing hepatitis B and/or D replication in hepatocytes as well as decreasing entry of hepatitis B viral particles and/or hepatitis D viral particles into hepatocytes using such inhibitors.

### BACKGROUND

Hepatitis B virus (HBV) infection is a major global public health problem (see, e.g., World Health Organization Hepatitis B fact sheet, 2021, available at: who.int/news-room/fact-sheets/detail/hepatitis-b). Globally, HBV is estimated to infect 296 million worldwide, and HDV is estimated to infect 48-60 million people. HBV results in the death of nearly 1 million people per year. HBV and HDV infect hepatocytes, and chronic HBV and HDV infections can cause severe liver disease. HDV depends on HBV to replicate and consequently only propagates when coinfecting with HBV.

Certain 1,5-benzothiazepine and 1,2,5-benzothiadiazepine derivatives are potent inhibitors of apical sodium-dependent bile acid transporter (ASBT) and/or Na+/taurocholate co-transporting polypeptide (NTCP; also known as liver bile acid transporter (LBAT)). Na+-taurocholate cotransporting polypeptide (NTCP) is a bile acid (BA) transporter at the hepatocyte-sinusoidal membrane. NTCP mediates uptake of bile acids into hepatocytes. NTCP also acts as a host receptor for hepatitis B and D viruses (HBV/HDV). Pharmacologic inhibition of NTCP may represent an approach for preventing HBV and HDV infection.

### SUMMARY

Provided herein are methods for treating hepatitis B (HBV) in a subject in need thereof, the method comprising orally administering to the subject a therapeutically effective amount of (Z)-3-((3-butyl-2- methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

Also provided herein are methods for preventing or decreasing entry of a hepatitis B viral particle into a hepatocyte in a subject in need thereof, the method comprising administering to the subject a therapeutically effective amount of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

Also provided herein are methods for decreasing hepatitis B viral replication in a hepatocyte in a subject in need thereof, the method comprising administering to the subject a therapeutically effective amount of (Z)-3-((3-butyl-2-methyl-7-(methylthio)-1,1-dioxido-5-phenyl-2,3,4,5-tetrahydro -1,2,5-benzothiadiazepin-8-yl)oxy)-2-fluoroacrylic acid, or a pharmaceutically acceptable salt thereof.

In some embodiments, the subject has hepatitis D.

Also provided herein are methods for preventing hepatitis D infection in a subject having hepatitis B, the method comprising orally administering to the subject a therapeutically effective amount of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

Also provided herein are methods for treating hepatitis D (HDV) in a subject in need thereof, the method comprising orally administering to the subject a therapeutically effective amount of (Z)-3-((3-butyl-2- methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

Also provided herein are methods for preventing or decreasing entry of a hepatitis D viral particle into a hepatocyte in a subject in need thereof, the method comprising administering to the subject a therapeutically effective amount of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

Also provided herein are methods for decreasing hepatitis D viral replication in a hepatocyte in a subject in need thereof, the method comprising administering to the subject a therapeutically effective amount of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

In some embodiments, the method further comprises administering an additional anti-viral agent.

Also provided herein are methods for treating HBV in a subject in need thereof, the method comprising orally administering to the subject a therapeutically effective amount of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, and an additional anti-viral agent.

Also provided herein are methods method for treating HDV in a subject in need thereof, the method comprising orally administering to the subject a therapeutically effective amount of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, and an additional anti-viral agent.

In some embodiments, the additional anti-viral agent is selected from the group consisting of: entecavir, tenofovir, tenofovir disoproxil, tenofovir alafenamide, lamivudine, adefovir, adefovir dipivoxil, telbivudine, bulevirtide, an interferon, and a combination thereof. In some embodiments, the interferon is pegylated interferon, interferon alpha, or a combination thereof. In some embodiments, the additional anti-viral agent is tenofovir disoproxil.

In some embodiments, the subject has hepatitis B. In some embodiments, the subject has chronic hepatitis B. In some embodiments, the subject has chronic hepatitis D.

In some embodiments, the concentration of one or more biomarkers selected from HBV DNA, hepatitis B surface antigen (HBsAg), hepatitis B core antigen (HBcAg), hepatitis B e antigen (HBeAg), HDV DNA, and hepatitis D antigen (HDAg) in the serum of the subject decreases after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

In some embodiments, the concentration of HBV DNA in the serum of the subject decreases after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HBV DNA is determined in a serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HBV DNA in the serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, is decreased as compared to a reference concentration of HBV DNA. In some embodiments, the reference concentration of HBV DNA is a level of HBV DNA in a serum sample obtained from the subject prior to administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

In some embodiments, the concentration of HBV DNA in the serum of the subject is decreased by about 10% to about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HBV DNA in the serum of the subject is decreased by about 5%, about 10%, about 25%, about 50%, about 75%, or about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HBV DNA in the serum of the subject is undetectable after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

In some embodiments, the concentration of hepatitis B surface antigen (HBsAg) in the serum of the subject decreases after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HBsAg is determined in a serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HBsAg in the serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, is decreased as compared to a reference concentration of HBsAg. In some embodiments, the reference concentration of HBsAg is a concentration of HBsAg in a serum sample obtained from the subject prior to administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

In some embodiments, the concentration of HBsAg in the serum of the subject is decreased by about 10% to about to about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HBsAg in the serum of the subject is decreased by about 5%, about 10%, about 25%, about 50%, about 75%, or about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HBsAg in the serum of the subject is undetectable after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

In some embodiments, the concentration of hepatitis B core antigen (HBcAg) in the serum of the subject decreases after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HBcAg is determined in a sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HBcAg in the serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, is decreased as compared to a reference concentration of HBcAg. In some embodiments, the reference concentration of HBcAg is a concentration of HBcAg in a serum sample obtained from the subject prior to administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

In some embodiments, the concentration of HBcAg in the serum of the subject is decreased by about 10% to about to about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HBcAg in the serum of the subject is decreased by about 5%, about 10%, about 25%, about 50%, about 75%, or about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HBcAg in the serum of the subject is undetectable after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

In some embodiments, the concentration of hepatitis B e antigen (HBeAg) in the serum of the subject decreases after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HBeAg is determined in a serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HBeAg in the serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, is decreased as compared to a reference concentration of HBeAg. In some embodiments, the reference concentration of HBeAg is a concentration of HBeAg in a serum sample obtained from the subject prior to administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

In some embodiments, the concentration of HBeAg in the serum of the subject is decreased by about 10% to about to about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HBeAg in the serum of the subject is decreased by about 5%, about 10%, about 25%, about 50%, about 75%, or about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HBeAg in the serum of the subject is undetectable after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

In some embodiments, the concentration of HDV DNA in the serum of the subject decreases after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HDV DNA is determined in a serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HDV DNA in the serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl) oxy)-2-fluoroacrylic acid, or a pharmaceutically acceptable salt thereof, is decreased as compared to a reference concentration of HDV DNA. In some embodiments, the reference concentration of HDV DNA is a concentration of HDV DNA in a serum sample obtained from the subject prior to administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

In some embodiments, the concentration of HDV DNA in the serum of the subject is decreased by about 10% to about to about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HDV DNA in the serum of the subject is decreased by about 5%, about 10%, about 25%, about 50%, about 75%, or about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HDV DNA in the serum of the subject is undetectable after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

In some embodiments, the concentration of hepatitis D antigen (HDAg) in the serum of the subject decreases after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HDAg is determined in a serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HDAg in the serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, is decreased as compared to a reference concentration of HDAg. In some embodiments, n the reference concentration of HDAg is a concentration of HDAg in a serum sample obtained from the subject prior to administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

In some embodiments, the concentration of HDAg in the serum of the subject is decreased by about 10% to about to about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HDAg in the serum of the subject is decreased by about 5%, about 10%, about 25%, about 50%, about 75%, or about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof. In some embodiments, the concentration of HDAg in the serum of the subject is undetectable after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

In some embodiments, the subject is administered (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, within 18 hours of exposure to hepatitis B. In some embodiments, the subject is administered (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, within 18 hours of exposure to hepatitis D.

In some embodiments, a therapeutically effective amount of (R)-(Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, is administered to the subject.

In some embodiments, a therapeutically effective amount of (S)-(Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, is administered to the subject.

The details of one or more embodiments of the disclosure are set forth in the accompanying drawings and the description below. Other features, objects, and advantages of the invention will be apparent from the description and drawings, and from the claims. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

### DESCRIPTION OF DRAWINGS

FIG. 1 is a schematic depicting NTCP in the enterohepatic circulation and HBV/HDV entry receptor.

FIG. 2A is a plot showing the Kᵢ values for NTCP.

FIG. 2B is a plot showing the Kᵢ values for ASBT.

FIG. 3A is a plot showing inhibition of NTCP with Myrcludex B after washout. Mean (SD) values (n=3) are depicted.

FIG. 3B is a plot showing inhibition of NTCP with Compound 1 after washout. Mean (SD) values (n=3) are depicted.

FIGS. 4A and 4B are plots showing inhibition of bile acid transport in NTCP-expressing HEK293 cells. Mean (SD) values for quadruplicate determinations from a representative experiment are depicted. *P<0.01 vs control by Student's t-test.

FIGS. 4C and 4D are plots showing inhibition of preS1 peptide binding in NTCP-expressing HEK293 cells. Mean (SD) values for quadruplicate determinations from a representative experiment are depicted. *P<0.01 vs control by Student's t-test.

FIGS. 5A and 5B are plots showing inhibition of M. fascicularis Ntcp.

FIG. 6 is a plot showing Compound 1 cytotoxicity in HepG2 cells expressing NTCP.

FIG. 7A is a plot showing Compound 1 inhibition of HBV in HepG2 cells expressing NTCP.

FIG. 7B is a plot showing Compound 1 inhibition of HDV in HepG2 cells expressing NTCP.

FIG. 7C is a plot showing Compound 1 inhibition of HBV in HepG2 cells expressing NTCP.

FIGS. 7D and 7E are plots showing Compound 1 inhibition of in vitro HBV infection via secreted HBeAg. Data represent means ±SD of triplicates; 50%: 50% of control (w/o inhibitor); significantly different from uninhibited control with p>0.05 (ns), p≤0.05 (*), p≤0.01 (**), p≤0.001 (***), p≤0.0001 (****).

FIGS. 7F and 7G are plots showing Compound 1 inhibition of in vitro HBV infection via HBc-positive cells. Data represent means±SD of triplicates; 50%: 50% of control (w/o inhibitor); significantly different from uninhibited control with p>0.05 (ns), p≤0.05 (*), p≤0.01 (**), p≤0.001 (***), p≤0.0001 (****).

FIG. 8A is a plot showing inhibition of HBV infection with Compound 1 or tenofovir disoproxil fumarate (TDF) in primary human hepatocytes when given 18 hours pre-infection with HBV. Mean values shown (n=3).

FIG. 8B is a plot showing inhibition of HBV infection with Compound 1 or tenofovir disoproxil fumarate (TDF) in primary human hepatocytes when given 18 hours post-infection with HBV. Mean values shown (n=3).

FIG. 8C is a plot showing cytotoxicity of Compound 1 or tenofovir disoproxil fumarate (TDF) in primary human hepatocytes. Mean values shown (n=3).

FIG. 8D is a plot showing inhibition of HBV infection with Compound 1 at various concentrations in combination with TDF.

FIG. 9 is a plot showing a summary of antiviral data with Compound 1. Mean values, n=3. Line represents a non-linear regression fit to the merged data from the three different studies. Data point from 10-11 M concentration in Study #01 was excluded for curve fitting purposes.

FIG. 10 is a plot showing pharmacokinetics of Compound 1 in humanized mice. Mean (SEM) values (n=3) are depicted. p.o., orally.

FIG. 11A is a plot showing serum HBV DNA concentrations in humanized mice during and after treatment with Compound 1.

FIG. 11B is a plot showing serum HBV DNA concentrations in humanized mice during treatment with Compound 1.

FIG. 12A is a plot showing serum HBsAg concentrations in humanized mice during and after treatment with Compound 1.

FIG. 12B is a plot showing serum HBsAg concentrations in humanized mice during treatment with Compound 1.

FIG. 12C is a plot showing serum HBeAg concentrations in humanized mice during treatment with Compound 1.

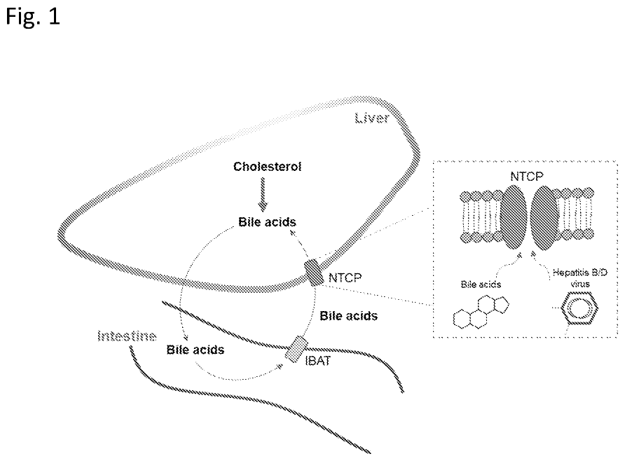

### DETAILED DESCRIPTION

HBV and HDV use Na+-taurocholate cotransporting polypeptide (NTCP also called liver bile acid transporter (LBAT); gene symbol SLC10A1) to enter and infect human hepatocytes. See, Yan H, et al. eLife. 2012;1:e00049. NTCP and other members of the SLC10 family of solute carrier proteins, such as the apical sodium dependent bile acid transporter (ASBT, also called ileal bile acid transporter (IBAT), ISBT, ABAT or NTCP2; gene symbol SLC10A2), control the transport of bile acids in the human body. In the liver, bile acids are efficiently extracted from portal blood by the liver bile acid transporter (LBAT) and re-secreted across the canalicular membrane by the bile salt export pump (BSEP; gene symbol ABCB11). The reabsorption of bile acids in the ileum is handled by the apical sodium-dependent bile acid transporter (ASBT), where it is commonly referred to as ileal bile acid transporter (IBAT). Both NTCP and ASBT function as electrogenic sodium-solute cotransporters that move two or more Na+ ions per molecule of solute.

LBAT also functions as a cellular receptor for viral entry of the hepatitis B virus (HBV) and hepatitis D virus (HDV), which in turn is a major cause of liver disease and hepatocellular carcinoma. NTCP interacts with a key region in the pre-S1 domain of the HBV envelope L protein. Yan H, et al. eLife. 2012;1:e00049. It has been suggested that residues 157 to 165 of NTCP are important for binding to the receptor-binding region of the preS1 domain of the L protein of HBV and that these residues contribute to NTCP-mediated HBV and HDV infections. Yan H, et al. eLife. 2012;1:e00049.

Provided herein are methods of treating hepatitis B (HBV) and/or hepatitis D (HDV) in a subject in need thereof, comprising administering to the subject one or more NTCP inhibitors (e.g., any of the NTCP inhibitors described herein). Also provided herein are methods for preventing or decreasing entry of a hepatitis B viral particle and/or hepatitis D viral particle into a hepatocyte in a subject in need thereof, comprising administering to the subject one or more NTCP inhibitors (e.g., any of the NTCP inhibitors described herein). Also provided herein are methods for decreasing hepatitis B and/or hepatitis D viral replication in a hepatocyte in a subject in need thereof, comprising administering to the subject one or more NTCP inhibitors (e.g., any of the NTCP inhibitors described herein).

An “NTCP inhibitor” as used herein includes any compound that exhibits NTCP inactivation activity (e.g., inhibiting or decreasing). In some embodiments, the NTCP inhibitor reduces NTCP activity. In some embodiments, the NTCP inhibitor prevents or reduces production and/or function of NTCP. In some embodiments, an NTCP inhibitor is a dual inhibitor, i.e., it inhibits NTCP as well as IBAT. In some embodiments, an NTCP inhibitor is selective for NTCP over other members of the SLC10 family of solute carrier proteins, such as IBAT. In some embodiments, an NTCP inhibitor has a molecular weight of less than about 1,000 g/mol.

The ability of compounds to act as inhibitors of NTCP may be demonstrated by assays known in the art. The activity of the compounds and compositions provided herein as NTCP inhibitors can be assayed in vitro, in vivo, or in a cell line. In vitro assays include assays that determine inhibition of bile salt transport by NTCP. Assays can include, for example, those described in U.S. Pat. No. 11,180,465.

NTCP inhibitors as described herein include compounds of formula (I):

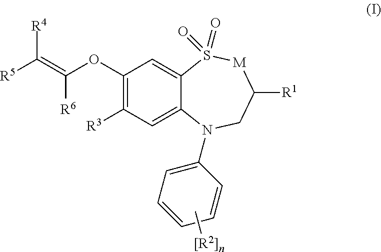

wherein
M is selected from —CH₂— and —NR⁷—;
R¹ is C₁₋₄ alkyl;
R² is independently selected from the group consisting of hydrogen, halogen, hydroxy, C₁₋₄ alkyl, C₁₋₄ haloalkyl, C₁₋₄ alkoxy, cyano, nitro, amino, N-(C₁₋₄ alkyl)amino, N,N-di(C₁₋₄ alkyl)amino, N-(aryl-C₁₋₄ alkyl)amino, C₁₋₆ alkylcarbonylamino, C₃₋₆ cycloalkylcarbonylamino, N-(C₁₋₄ alkyl)amino-carbonyl, N,N-di(C₁₋₄ alkyl)aminocarbonyl, C₁₋₄ alkyloxycarbonylamino, C₃₋₆ cycloalkyloxycarbonylamino, C₁₋₄ alkylsulfonamido and C₃₋₆ cycloalkylsulfonamido;
n is an integer 1, 2 or 3;
R³ is selected from the group consisting of hydrogen, halogen, cyano, C₁₋₄ alkyl, C₃₋₆ cycloalkyl, C₁₋₄ alkoxy, C₃₋₆ cycloalkyloxy, C₁₋₄ alkylthio, C₃₋₆ cycloalkylthio, amino, N-(C₁₋₄ alkyl)amino and N,N-di(C₁₋₄ alkyl)amino;
one of R⁴ and R⁵ is carboxyl, and the other of R⁴ and R⁵ is selected from the group consisting of hydrogen, fluoro, C₁₋₄ alkyl and C₁₋₄ haloalkyl;
R⁶ is selected from the group consisting of hydrogen and C₁₋₄ alkyl; and
R⁷ is selected from the group consisting of hydrogen and C₁₋₄ alkyl;
   or a pharmaceutically acceptable salt thereof.

In some embodiments, R¹ is C₂₋₄ alkyl. In some embodiments, R¹ is n-propyl. In some embodiments, R¹ is n-butyl.

In some embodiments, R² is selected from the group consisting of hydrogen, fluoro, chloro, bromo, hydroxy, methoxy, amino, methylamino, dimethylamino, isopropylcarbonylamino, tert-butylcarbonylamino, tert-butylaminocarbonyl, tert-butoxycarbonylamino, methylsulfonamido and cyclopropylsulfonamido. In some embodiments, n is 1, i.e. the phenyl-ring is substituted with only one substituent R². In some embodiments, R² is in the para-position.

In some embodiments, R³ is selected from the group consisting of hydrogen, fluoro, chloro, bromo, methyl, cyclopropyl, methoxy, ethoxy, methylthio, ethylthio, amino, methylamino and dimethylamino.

In some embodiments, R⁴ is hydrogen or fluoro.

In some embodiments, R⁵ is carboxyl.

In some embodiments, R⁶ is hydrogen.

In some embodiments, R⁷ is hydrogen or methyl.

In some embodiments, the compound of formula (I) is a compound of formula (I-a):

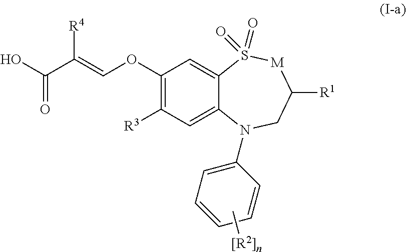

wherein
M is selected from the group consisting of —CH₂—, —NH— and —NCH₃—;
R¹ is C₂₋₄ alkyl;
R² is independently selected from the group consisting of hydrogen, halogen, hydroxy, C₁₋₄ alkyl, C₁₋₄ haloalkyl, C₁₋₄ alkoxy, amino, N-(C₁₋₄ alkyl)amino, N,N-di(C₁₋₄ alkyl)amino, C₁₋₆ alkylcarbonylamino, C₃₋₆ cycloalkylcarbonylamino, N-(C₁₋₄ alkyl)aminocarbonyl, N,N-di(C₁₋₄ alkyl)aminocarbonyl, C₁₋₄ alkyloxycarbonylamino, C₁₋₄ alkylsulfonamido, and C₃₋₆ cycloalkylsulfonamido;
n is an integer 1 or 2;
R³ is selected from the group consisting of hydrogen, halogen, C₁₋₄ alkyl, C₃₋₆ cycloalkyl, C₁₋₄ alkoxy, C₁₋₄ alkylthio, amino, N-(C₁₋₄ alkyl)amino and N,N-di(C₁₋₄ alkyl)amino;
R⁴ is hydrogen or fluoro;
   or a pharmaceutically acceptable salt thereof.

In some embodiments, the compound of formula (I) is a compound of formula (I-b):

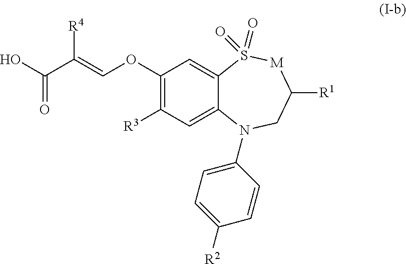

wherein
M is selected from the group consisting of —CH₂—, —NH— and —NCH₃—;
R¹ is C₂₋₄ alkyl, more preferably n-propyl or n-butyl;
R² is independently selected from the group consisting of hydrogen, fluoro, chloro, bromo, hydroxy, methoxy, amino, methylamino, dimethylamino, isopropylcarbonylamino, tert-butylcarbonylamino, tert-butylaminocarbonyl, tert-butoxycarbonylamino, methylsulfonamido and cyclopropylsulfonamido;
R³ is selected from the group consisting of fluoro, chloro, bromo, methyl, cyclopropyl, methoxy, ethoxy, methylthio, ethylthio, amino, methylamino and dimethylamino;
R⁴ is hydrogen or fluoro;
   or a pharmaceutically acceptable salt thereof.

In some embodiments, the compound of formula (I) is a compound of formula (I-b), as defined above, wherein M and R¹ to R⁴ are as indicated in Table 1 below, or a pharmaceutically acceptable salt thereof:

TABLE 1

| M | R¹ | R² | R³ | R⁴ |
|---|---|---|---|---|
| CH₂ | CH₂CH₂CH₃ | H | SCH₃ | H |
| CH₂ | CH₂CH₂CH₃ | H | SCH₃ | F |
| CH₂ | CH₂CH₂CH₃ | F | SCH₃ | H |
| CH₂ | CH₂CH₂CH₃ | F | SCH₃ | F |
| NH | CH₂CH₂CH₃ | H | SCH₃ | H |
| NH | CH₂CH₂CH₃ | H | SCH₃ | F |
| NH | CH₂CH₂CH₃ | F | SCH₃ | H |
| NH | CH₂CH₂CH₃ | F | SCH₃ | F |
| NCH₃ | CH₂CH₂CH₃ | H | SCH₃ | H |
| NCH₃ | CH₂CH₂CH₃ | H | SCH₃ | F |
| NCH₃ | CH₂CH₂CH₃ | F | SCH₃ | H |
| NCH₃ | CH₂CH₂CH₃ | F | SCH₃ | F |
| CH₂ | CH₂CH₂CH₃ | H | SCH₂CH₃ | H |
| CH₂ | CH₂CH₂CH₃ | H | SCH₂CH₃ | F |
| CH₂ | CH₂CH₂CH₃ | F | SCH₂CH₃ | H |
| CH₂ | CH₂CH₂CH₃ | F | SCH₂CH₃ | F |
| NH | CH₂CH₂CH₃ | H | SCH₂CH₃ | H |
| NH | CH₂CH₂CH₃ | H | SCH₂CH₃ | F |
| NH | CH₂CH₂CH₃ | F | SCH₂CH₃ | H |
| NH | CH₂CH₂CH₃ | F | SCH₂CH₃ | F |
| NCH₃ | CH₂CH₂CH₃ | H | SCH₂CH₃ | H |
| NCH₃ | CH₂CH₂CH₃ | H | SCH₂CH₃ | F |
| NCH₃ | CH₂CH₂CH₃ | F | SCH₂CH₃ | H |
| NCH₃ | CH₂CH₂CH₃ | F | SCH₂CH₃ | F |
| CH₂ | CH₂CH₂CH₃ | H | Cl | H |
| CH₂ | CH₂CH₂CH₃ | H | Cl | F |
| CH₂ | CH₂CH₂CH₃ | F | Cl | H |
| CH₂ | CH₂CH₂CH₃ | F | Cl | F |
| NH | CH₂CH₂CH₃ | H | Cl | H |
| NH | CH₂CH₂CH₃ | H | Cl | F |
| NH | CH₂CH₂CH₃ | F | Cl | H |
| NH | CH₂CH₂CH₃ | F | Cl | F |
| NCH₃ | CH₂CH₂CH₃ | H | Cl | H |
| NCH₃ | CH₂CH₂CH₃ | H | Cl | F |
| NCH₃ | CH₂CH₂CH₃ | F | Cl | H |
| NCH₃ | CH₂CH₂CH₃ | F | Cl | F |
| CH₂ | CH₂CH₂CH₃ | H | F | H |
| CH₂ | CH₂CH₂CH₃ | H | F | F |
| CH₂ | CH₂CH₂CH₃ | F | F | H |
| CH₂ | CH₂CH₂CH₃ | F | F | F |
| NH | CH₂CH₂CH₃ | H | F | H |
| NH | CH₂CH₂CH₃ | H | F | F |
| NH | CH₂CH₂CH₃ | F | F | H |
| NH | CH₂CH₂CH₃ | F | F | F |
| NCH₃ | CH₂CH₂CH₃ | H | F | H |
| NCH₃ | CH₂CH₂CH₃ | H | F | F |
| NCH₃ | CH₂CH₂CH₃ | F | F | H |
| NCH₃ | CH₂CH₂CH₃ | F | F | F |
| CH₂ | CH₂CH₂CH₃ | H | Br | H |
| CH₂ | CH₂CH₂CH₃ | H | Br | F |
| CH₂ | CH₂CH₂CH₃ | F | Br | H |
| CH₂ | CH₂CH₂CH₃ | F | Br | F |
| NH | CH₂CH₂CH₃ | H | Br | H |
| NH | CH₂CH₂CH₃ | H | Br | F |
| NH | CH₂CH₂CH₃ | F | Br | H |
| NH | CH₂CH₂CH₃ | F | Br | F |
| NCH₃ | CH₂CH₂CH₃ | H | Br | H |
| NCH₃ | CH₂CH₂CH₃ | H | Br | F |
| NCH₃ | CH₂CH₂CH₃ | F | Br | H |
| NCH₃ | CH₂CH₂CH₃ | F | Br | F |
| CH₂ | CH₂CH₂CH₃ | H | N(CH₃)₂ | H |
| CH₂ | CH₂CH₂CH₃ | H | N(CH₃)₂ | F |
| CH₂ | CH₂CH₂CH₃ | F | N(CH₃)₂ | H |
| CH₂ | CH₂CH₂CH₃ | F | N(CH₃)₂ | F |
| NH | CH₂CH₂CH₃ | H | N(CH₃)₂ | H |
| NH | CH₂CH₂CH₃ | H | N(CH₃)₂ | F |
| NH | CH₂CH₂CH₃ | F | N(CH₃)₂ | H |
| NH | CH₂CH₂CH₃ | F | N(CH₃)₂ | F |
| NCH₃ | CH₂CH₂CH₃ | H | N(CH₃)₂ | H |
| NCH₃ | CH₂CH₂CH₃ | H | N(CH₃)₂ | F |
| NCH₃ | CH₂CH₂CH₃ | F | N(CH₃)₂ | H |
| NCH₃ | CH₂CH₂CH₃ | F | N(CH₃)₂ | F |
| CH₂ | CH₂CH₂CH₂CH₃ | H | SCH₃ | H |
| CH₂ | CH₂CH₂CH₂CH₃ | H | SCH₃ | F |
| CH₂ | CH₂CH₂CH₂CH₃ | F | SCH₃ | H |
| CH₂ | CH₂CH₂CH₂CH₃ | F | SCH₃ | F |
| NH | CH₂CH₂CH₂CH₃ | H | SCH₃ | H |
| NH | CH₂CH₂CH₂CH₃ | H | SCH₃ | F |
| NH | CH₂CH₂CH₂CH₃ | F | SCH₃ | H |
| NH | CH₂CH₂CH₂CH₃ | F | SCH₃ | F |
| NCH₃ | CH₂CH₂CH₂CH₃ | H | SCH₃ | H |
| NCH₃ | CH₂CH₂CH₂CH₃ | H | SCH₃ | F |
| NCH₃ | CH₂CH₂CH₂CH₃ | F | SCH₃ | H |
| NCH₃ | CH₂CH₂CH₂CH₃ | F | SCH₃ | F |
| CH₂ | CH₂CH₂CH₂CH₃ | H | SCH₂CH₃ | H |
| CH₂ | CH₂CH₂CH₂CH₃ | H | SCH₂CH₃ | F |
| CH₂ | CH₂CH₂CH₂CH₃ | F | SCH₂CH₃ | H |
| CH₂ | CH₂CH₂CH₂CH₃ | F | SCH₂CH₃ | F |
| NH | CH₂CH₂CH₂CH₃ | H | SCH₂CH₃ | H |
| NH | CH₂CH₂CH₂CH₃ | H | SCH₂CH₃ | F |
| NH | CH₂CH₂CH₂CH₃ | F | SCH₂CH₃ | H |
| NH | CH₂CH₂CH₂CH₃ | F | SCH₂CH₃ | F |
| NCH₃ | CH₂CH₂CH₂CH₃ | H | SCH₂CH₃ | H |
| NCH₃ | CH₂CH₂CH₂CH₃ | H | SCH₂CH₃ | F |
| NCH₃ | CH₂CH₂CH₂CH₃ | F | SCH₂CH₃ | H |
| NCH₃ | CH₂CH₂CH₂CH₃ | F | SCH₂CH₃ | F |
| CH₂ | CH₂CH₂CH₂CH₃ | H | Cl | H |
| CH₂ | CH₂CH₂CH₂CH₃ | H | Cl | F |
| CH₂ | CH₂CH₂CH₂CH₃ | F | Cl | H |
| CH₂ | CH₂CH₂CH₂CH₃ | F | Cl | F |
| NH | CH₂CH₂CH₂CH₃ | H | Cl | H |
| NH | CH₂CH₂CH₂CH₃ | H | Cl | F |
| NH | CH₂CH₂CH₂CH₃ | F | Cl | H |
| NH | CH₂CH₂CH₂CH₃ | F | Cl | F |
| NCH₃ | CH₂CH₂CH₂CH₃ | H | Cl | H |
| NCH₃ | CH₂CH₂CH₂CH₃ | H | Cl | F |
| NCH₃ | CH₂CH₂CH₂CH₃ | F | Cl | H |
| NCH₃ | CH₂CH₂CH₂CH₃ | F | Cl | F |
| CH₂ | CH₂CH₂CH₂CH₃ | H | F | H |
| CH₂ | CH₂CH₂CH₂CH₃ | H | F | F |
| CH₂ | CH₂CH₂CH₂CH₃ | F | F | H |
| CH₂ | CH₂CH₂CH₂CH₃ | F | F | F |
| NH | CH₂CH₂CH₂CH₃ | H | F | H |
| NH | CH₂CH₂CH₂CH₃ | H | F | F |
| NH | CH₂CH₂CH₂CH₃ | F | F | H |
| NH | CH₂CH₂CH₂CH₃ | F | F | F |
| NCH₃ | CH₂CH₂CH₂CH₃ | H | F | H |
| NCH₃ | CH₂CH₂CH₂CH₃ | H | F | F |
| NCH₃ | CH₂CH₂CH₂CH₃ | F | F | H |
| NCH₃ | CH₂CH₂CH₂CH₃ | F | F | F |
| CH₂ | CH₂CH₂CH₂CH₃ | H | Br | H |
| CH₂ | CH₂CH₂CH₂CH₃ | H | Br | F |
| CH₂ | CH₂CH₂CH₂CH₃ | F | Br | H |
| CH₂ | CH₂CH₂CH₂CH₃ | F | Br | F |
| NH | CH₂CH₂CH₂CH₃ | H | Br | H |
| NH | CH₂CH₂CH₂CH₃ | H | Br | F |
| NH | CH₂CH₂CH₂CH₃ | F | Br | H |
| NH | CH₂CH₂CH₂CH₃ | F | Br | F |
| NCH₃ | CH₂CH₂CH₂CH₃ | H | Br | H |
| NCH₃ | CH₂CH₂CH₂CH₃ | H | Br | F |
| NCH₃ | CH₂CH₂CH₂CH₃ | F | Br | H |
| NCH₃ | CH₂CH₂CH₂CH₃ | F | Br | F |
| CH₂ | CH₂CH₂CH₂CH₃ | H | N(CH₃)₂ | H |
| CH₂ | CH₂CH₂CH₂CH₃ | H | N(CH₃)₂ | F |
| CH₂ | CH₂CH₂CH₂CH₃ | F | N(CH₃)₂ | H |
| CH₂ | CH₂CH₂CH₂CH₃ | F | N(CH₃)₂ | F |
| NH | CH₂CH₂CH₂CH₃ | H | N(CH₃)₂ | H |
| NH | CH₂CH₂CH₂CH₃ | H | N(CH₃)₂ | F |
| NH | CH₂CH₂CH₂CH₃ | F | N(CH₃)₂ | H |
| NH | CH₂CH₂CH₂CH₃ | F | N(CH₃)₂ | F |
| NCH₃ | CH₂CH₂CH₂CH₃ | H | N(CH₃)₂ | H |
| NCH₃ | CH₂CH₂CH₂CH₃ | H | N(CH₃)₂ | F |
| NCH₃ | CH₂CH₂CH₂CH₃ | F | N(CH₃)₂ | H |
| NCH₃ | CH₂CH₂CH₂CH₃ | F | N(CH₃)₂ | F |
| CH₂ | CH₂CH₂CH₂CH₃ | C═ONHC(CH₃)₃ | SCH₃ | H |
| CH₂ | CH₂CH₂CH₂CH₃ | NHC═OC(CH₃)₃ | SCH₃ | F |
| CH₂ | CH₂CH₂CH₂CH₃ | NHC═OCH(CH₃)₂ | SCH₃ | H |
| CH₂ | CH₂CH₂CH₂CH₃ | Cl | SCH₃ | F |
| NH | CH₂CH₂CH₂CH₃ | C═ONHC(CH₃)₃ | SCH₃ | H |
| NH | CH₂CH₂CH₂CH₃ | NHC═OC(CH₃)₃ | SCH₃ | F |
| NH | CH₂CH₂CH₂CH₃ | NHC═OCH(CH₃)₂ | SCH₃ | H |
| NH | CH₂CH₂CH₂CH₃ | Cl | SCH₃ | F |
| NCH₃ | CH₂CH₂CH₂CH₃ | C═ONHC(CH₃)₃ | SCH₃ | H |
| NCH₃ | CH₂CH₂CH₂CH₃ | NHC═OC(CH₃)₃ | SCH₃ | F |
| NCH₃ | CH₂CH₂CH₂CH₃ | NHC═OCH(CH₃)₂ | SCH₃ | H |
| NCH₃ | CH₂CH₂CH₂CH₃ | Cl | SCH₃ | F |
| CH₂ | CH₂CH₂CH₂CH₃ | C═ONHC(CH₃)₃ | SCH₂CH₃ | H |
| CH₂ | CH₂CH₂CH₂CH₃ | NHC═OC(CH₃)₃ | SCH₂CH₃ | F |
| CH₂ | CH₂CH₂CH₂CH₃ | NHC═OCH(CH₃)₂ | SCH₂CH₃ | H |
| CH₂ | CH₂CH₂CH₂CH₃ | Cl | SCH₂CH₃ | F |
| NH | CH₂CH₂CH₂CH₃ | C═ONHC(CH₃)₃ | SCH₂CH₃ | H |
| NH | CH₂CH₂CH₂CH₃ | NHC═OC(CH₃)₃ | SCH₂CH₃ | F |
| NH | CH₂CH₂CH₂CH₃ | NHC═OCH(CH₃)₂ | SCH₂CH₃ | H |
| NH | CH₂CH₂CH₂CH₃ | Cl | SCH₂CH₃ | F |
| NCH₃ | CH₂CH₂CH₂CH₃ | C═ONHC(CH₃)₃ | SCH₂CH₃ | H |
| NCH₃ | CH₂CH₂CH₂CH₃ | NHC═OC(CH₃)₃ | SCH₂CH₃ | F |
| NCH₃ | CH₂CH₂CH₂CH₃ | NHC═OCH(CH₃)₂ | SCH₂CH₃ | H |
| NCH₃ | CH₂CH₂CH₂CH₃ | Cl | SCH₂CH₃ | F |
| CH₂ | CH₂CH₂CH₂CH₃ | C═ONHC(CH₃)₃ | Cl | H |
| CH₂ | CH₂CH₂CH₂CH₃ | NHC═OC(CH₃)₃ | Cl | F |
| CH₂ | CH₂CH₂CH₂CH₃ | NHC═OCH(CH₃)₂ | Cl | H |
| CH₂ | CH₂CH₂CH₂CH₃ | Cl | Cl | F |
| NH | CH₂CH₂CH₂CH₃ | C═ONHC(CH₃)₃ | Cl | H |
| NH | CH₂CH₂CH₂CH₃ | NHC═OC(CH₃)₃ | Cl | F |
| NH | CH₂CH₂CH₂CH₃ | NHC═OCH(CH₃)₂ | Cl | H |
| NH | CH₂CH₂CH₂CH₃ | Cl | Cl | F |
| NCH₃ | CH₂CH₂CH₂CH₃ | C═ONHC(CH₃)₃ | Cl | H |
| NCH₃ | CH₂CH₂CH₂CH₃ | NHC═OC(CH₃)₃ | Cl | F |
| NCH₃ | CH₂CH₂CH₂CH₃ | NHC═OCH(CH₃)₂ | Cl | H |
| NCH₃ | CH₂CH₂CH₂CH₃ | Cl | Cl | F |
| CH₂ | CH₂CH₂CH₂CH₃ | C═ONHC(CH₃)₃ | F | H |
| CH₂ | CH₂CH₂CH₂CH₃ | NHC═OC(CH₃)₃ | F | F |
| CH₂ | CH₂CH₂CH₂CH₃ | NHC═OCH(CH₃)₂ | F | H |
| CH₂ | CH₂CH₂CH₂CH₃ | Cl | F | F |
| NH | CH₂CH₂CH₂CH₃ | C═ONHC(CH₃)₃ | F | H |
| NH | CH₂CH₂CH₂CH₃ | NHC═OC(CH₃)₃ | F | F |
| NH | CH₂CH₂CH₂CH₃ | NHC═OCH(CH₃)₂ | F | H |
| NH | CH₂CH₂CH₂CH₃ | Cl | F | F |
| NCH₃ | CH₂CH₂CH₂CH₃ | C═ONHC(CH₃)₃ | F | H |
| NCH₃ | CH₂CH₂CH₂CH₃ | NHC═OC(CH₃)₃ | F | F |
| NCH₃ | CH₂CH₂CH₂CH₃ | NHC═OCH(CH₃)₂ | F | H |
| NCH₃ | CH₂CH₂CH₂CH₃ | Cl | F | F |
| CH₂ | CH₂CH₂CH₂CH₃ | C═ONHC(CH₃)₃ | Br | H |
| CH₂ | CH₂CH₂CH₂CH₃ | NHC═OC(CH₃)₃ | Br | F |
| CH₂ | CH₂CH₂CH₂CH₃ | NHC═OCH(CH₃)₂ | Br | H |
| CH₂ | CH₂CH₂CH₂CH₃ | Cl | Br | F |
| NH | CH₂CH₂CH₂CH₃ | C═ONHC(CH₃)₃ | Br | H |
| NH | CH₂CH₂CH₂CH₃ | NHC═OC(CH₃)₃ | Br | F |
| NH | CH₂CH₂CH₂CH₃ | NHC═OCH(CH₃)₂ | Br | H |
| NH | CH₂CH₂CH₂CH₃ | Cl | Br | F |
| NCH₃ | CH₂CH₂CH₂CH₃ | C═ONHC(CH₃)₃ | Br | H |
| NCH₃ | CH₂CH₂CH₂CH₃ | NHC═OC(CH₃)₃ | Br | F |
| NCH₃ | CH₂CH₂CH₂CH₃ | NHC═OCH(CH₃)₂ | Br | H |
| NCH₃ | CH₂CH₂CH₂CH₃ | Cl | Br | F |
| CH₂ | CH₂CH₂CH₂CH₃ | C═ONHC(CH₃)₃ | N(CH₃)₂ | H |
| CH₂ | CH₂CH₂CH₂CH₃ | NHC═OC(CH₃)₃ | N(CH₃)₂ | F |
| CH₂ | CH₂CH₂CH₂CH₃ | NHC═OCH(CH₃)₂ | N(CH₃)₂ | H |
| CH₂ | CH₂CH₂CH₂CH₃ | Cl | N(CH₃)₂ | F |
| NH | CH₂CH₂CH₂CH₃ | C═ONHC(CH₃)₃ | N(CH₃)₂ | H |
| NH | CH₂CH₂CH₂CH₃ | NHC═OC(CH₃)₃ | N(CH₃)₂ | F |
| NH | CH₂CH₂CH₂CH₃ | NHC═OCH(CH₃)₂ | N(CH₃)₂ | H |
| NH | CH₂CH₂CH₂CH₃ | Cl | N(CH₃)₂ | F |
| NCH₃ | CH₂CH₂CH₂CH₃ | C═ONHC(CH₃)₃ | N(CH₃)₂ | H |
| NCH₃ | CH₂CH₂CH₂CH₃ | NHC═OC(CH₃)₃ | N(CH₃)₂ | F |
| NCH₃ | CH₂CH₂CH₂CH₃ | NHC═OCH(CH₃)₂ | N(CH₃)₂ | H |
| NCH₃ | CH₂CH₂CH₂CH₃ | Cl | N(CH₃)₂ | F |

In some embodiments, the compound of formula (I) is selected from the group consisting of:
(E)-3-((3-butyl-7-(methylthio)-1,1-dioxido-5-phenyl-2,3,4,5-tetrahydro-1,5-benzothiazepin-8-yl)oxy)acrylic acid;
(R)-(E)-3-((3-butyl-7-(methylthio)-1,1-dioxido-5-phenyl-2,3,4,5-tetrahydro-1,5-benzothiazepin-8-yl)oxy)acrylic acid;
(S)-(E)-3-((3-butyl-7-(methylthio)-1,1-dioxido-5-phenyl-2,3,4,5-tetrahydro-1,5-benzothiazepin-8-yl)oxy)acrylic acid;
(Z)-3-((3-butyl-7-(methylthio)-1,1-dioxido-5-phenyl-2,3,4,5-tetrahydro-1,5-benzothiazepin-8-yl)oxy)-2-fluoroacrylic acid;
(S)-(Z)-3-((3-butyl-7-(methylthio)-1,1-dioxido-5-phenyl-2,3,4,5-tetrahydro-1,5-benzothiazepin-8-yl)oxy)-2-fluoroacrylic acid;
(R)-(Z)-3-((3-butyl-7-(methylthio)-1,1-dioxido-5-phenyl-2,3,4,5-tetrahydro-1,5-benzothiazepin-8-yl)oxy)-2-fluoroacrylic acid;
(Z)-3-((3-ethyl-7-(methylthio)-1,1-dioxido-5-phenyl-2,3,4,5-tetrahydro-1,5-benzothiazepin-8-yl)oxy)-2-fluoroacrylic acid;
(E)-3-((3-ethyl-7-(methylthio)-1,1-dioxido-5-phenyl-2,3,4,5-tetrahydro-1,5-benzothiazepin-8-yl)oxy)acrylic acid;
(E)-3-((3-butyl-7-(methylthio)-1,1-dioxido-5-phenyl-2,3,4,5-tetrahydro-1,2,5-benzothiadiazepin-8-yl)oxy)acrylic acid;
(Z)-3-((3-butyl-7-(methylthio)- 1,1-dioxido-5-phenyl-2,3,4,5-tetrahydro-1,2,5-benzothiadiazepin-8-yl)oxy)-2-fluoroacrylic acid;
(Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid;
(S)-(Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid;
(R)-(Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid;
(E)-3-((3-butyl-2-methyl-7-(methylthio)-1,1-dioxido-5-phenyl-2,3,4,5-tetrahydro-1,2,5-benzothiadiazepin-8-yl)oxy)acrylic acid;
(S)-(E)-3-((3-butyl-2-methyl-7-(methylthio)- 1,1-dioxido-5-phenyl-2,3,4,5-tetrahydro-1,2,5-benzothiadiazepin-8-yl)oxy)acrylic acid;
(R)-(E)-3-((3-butyl-2-methyl-7-(methylthio)- 1,1-dioxido-5-phenyl-2,3,4,5-tetrahydro-1,2,5-benzothiadiazepin-8-yl)oxy)acrylic acid;
(E)-3-((3-butyl-7-(ethylthio)-2-methyl-1,1-dioxido-5-phenyl-2,3,4,5-tetrahydro-1,2,5-benzothiadiazepin-8-yl)oxy)acrylic acid;
(S)-(E)-3-((3-butyl-7-(ethylthio)-2-methyl-1,1-dioxido-5-phenyl-2,3,4,5-tetrahydro-1,2,5-benzothiadiazepin-8-yl)oxy)acrylic acid;
(R)-(E)-3-((3-butyl-7-(ethylthio)-2-methyl-1,1-dioxido-5-phenyl-2,3,4,5-tetrahydro-1,2,5-benzothiadiazepin-8-yl)oxy)acrylic acid;
(E) -3-((3-butyl-5-(4-fluorophenyl)-2-methyl-7-(methylthio)-1,1-dioxido-2,3,4,5-tetrahydro-1,2,5-benzothiadiazepin-8-yl)oxy)acrylic acid;
(S)-(E)-3-((3-butyl-5-(4-fluorophenyl)-2-methyl-7-(methylthio)-1,1-dioxido-2,3,4,5-tetrahydro-1,2,5-benzothiadiazepin-8-yl)oxy)acrylic acid;
(R)-(E)-3-((3-butyl-5-(4-fluorophenyl)-2-methyl-7-(methylthio)-1,1-dioxido-2,3,4,5-tetrahydro-1,2,5-benzothiadiazepin-8-yl)oxy)acrylic acid;
(E)-3-((3-butyl-7-(ethylthio)-5-(4-fluorophenyl)-2-methyl-1,1-dioxido-2,3,4,5-tetrahydro-1,2,5-benzothiadiazepin-8-yl)oxy)acrylic acid;
(S)-(E)-3-((3-butyl-7-(ethylthio)-5-(4-fluorophenyl)-2-methyl-1,1-dioxido-2,3,4,5-tetrahydro-1,2,5-benzothiadiazepin-8-yl)oxy)acrylic acid;
(R)-(E) -3-((3-butyl-7-(ethylthio)-5-(4-fluorophenyl)-2-methyl-1,1-dioxido-2,3,4,5-tetrahydro-1,2,5-benzothiadiazepin-8-yl)oxy)acrylic acid;
(E)-3-((3-butyl-5-(4-fluorophenyl)-7-(methylthio)-1,1-dioxido-2,3,4,5-tetrahydro-1,5-benzothiazepin-8-yl)oxy)acrylic acid;
(S)-(E)-3-((3-butyl-5-(4-fluorophenyl)-7-(methylthio)-1,1-dioxido-2,3,4,5-tetrahydro-1,5-benzothiazepin-8-yl)oxy)acrylic acid;
(R)-(E)-3-((3-butyl-5-(4-fluorophenyl)-7-(methylthio)-1,1-dioxido-2,3,4,5-tetrahydro-1,5-benzothiazepin-8-yl)oxy)acrylic acid;
(E)-3-((3-Butyl-5-(4-fluorophenyl)-7-(methylthio)-1,1-dioxido-2,3,4,5-tetrahydro-1,2,5-benzothiadiazepin-8-yl)oxy)acrylic acid;
(E)-3-((3-Butyl-7-(ethylthio)-1,1-dioxido-5-phenyl-2,3,4,5-tetrahydro-1,5-benzothiazepin-8-yl)oxy)acrylic acid;
(E)-3-((3-Butyl-7-(ethylthio)-1,1-dioxido-5-phenyl-2,3,4,5-tetrahydro-1,2,5-benzothiadiazepin-8-yl)oxy)acrylic acid;
(E)-3-((3-Butyl-7-(ethylthio)-5-(4-orophenyl)-1,1-dioxido-2,3,4,5-tetrahydro-1,5-benzothiazepin-8-yl)oxy)acrylic acid;
(Z)-3-((3-butyl-5-(4-fluorophenyl)-7-(methylthio)-1,1-dioxido-2,3,4,5-tetrahydro-1,5-benzothiazepin-8-yl)oxy)-2-fluoroacrylic acid;
(Z)-3-((3-butyl-7-(ethylthio)-1,1-dioxido-5-phenyl-2,3,4,5-tetrahydro-1,5-benzothiazepin-8-yl)oxy)-2-fluoroacrylic acid;
(Z)-3-((3-butyl-7-(ethylthio)-5-(4-fluorophenyl)-1,1-dioxido-2,3,4,5-tetrahydro-1,5-benzothiazepin-8-yl)oxy)-2-fluoroacrylic acid;
(Z)-3-((3-butyl-5-(4-fluorophenyl)-2-methyl-7-(methylthio)-1,1-dioxido-2,3,4,5-tetrahydro-1,2,5-benzothiadiazepin-8-yl)oxy)-2-fluoroacrylic acid;
(Z)-3-((3-butyl-7-(ethylthio)-5-(4-fluorophenyl)-2-methyl-1,1-dioxido-2,3,4,5-tetrahydro-1,2,5-benzothiadiazepin-8-yl)oxy)-2-fluoroacrylic acid;
(Z)-3-((3-butyl-7-(ethylthio)-2-methyl-1,1-dioxido-5-phenyl-2,3,4,5-tetrahydro-1,2,5-benzothiadiazepin-8-yl)oxy)-2-fluoroacrylic acid;
(Z)-3-((3-butyl-5-(4-fluorophenyl)-7-(methylthio)-1,1-dioxido-2,3,4,5-tetrahydro-1,2,5-benzothiadiazepin-8-yl)oxy)-2-fluoroacrylic acid;
(Z)-3-((3-butyl-7-(ethylthio)-1,1-dioxido-5-phenyl-2,3,4,5-tetrahydro-1,2,5-benzothiadiazepin-8-yl)oxy)-2-fluoroacrylic acid;
(Z)-3-((3-butyl-7-(ethylthio)-5-(4-fluorophenyl)-1,1-dioxido-2,3,4,5-tetrahydro-1,2,5-benzothiadiazepin-8-yl)oxy)-2-fluoroacrylic acid; and
(E)-3-((3-butyl-7-(ethylthio)-5-(4-fluorophenyl)-1,1-dioxido-2,3,4,5-tetrahydro-1,2,5-benzothiadiazepin-8-yl)oxy)acrylic acid;
or a pharmaceutically acceptable salt thereof.

In some embodiments, the compound of formula (I) is (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid:

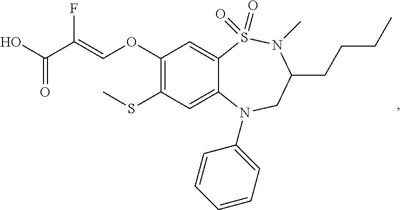 [SMILES: CCCCC1CN(c2ccccc2)c2cc(SC)c(O/C=C(\F)C(=O)O)cc2S(=O)(=O)N1C]

or a pharmaceutically acceptable salt thereof (Z)-3-((3-butyl-2-methyl-7-(methylthio)-1,1-dioxido-5-phenyl-2,3,4,5-tetrahydro-1,2,5-benzothiadiazepin-8-yl)oxy)-2 -fluoroacrylic acid is also referred to herein as “Compound 1.”

In some embodiments, the compound of formula (I) is (S)-(Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, is administered to the subject. In some embodiments, Compound 1 is (S)-(Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid.

In some embodiments, the compound of formula (I) is (R)-(Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, is administered to the subject. In some embodiments, Compound 1 is (R)-(Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid.

Compounds of formula (I) can be prepared as described in U.S. Pat. No. 11,180,465.

NTCP inhibitors as described herein also include a compound of formula (II)

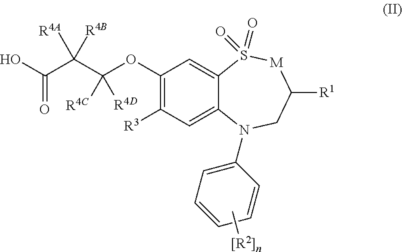

wherein
M is selected from —CH₂— and —NR⁵—;
R¹ is C₁₋₄ alkyl;
R² is independently selected from the group consisting of hydrogen, halogen, hydroxy, C₁₋₄ alkyl, C₁₋₄ haloalkyl, C₁₋₄ alkoxy, C₁₋₄ haloalkoxy, cyano, nitro, amino, N-(C₁₋₄ alkyl)amino, N,N-di(C₁₋₄ alkyl)amino, C₁₋₆ alkylcarbonylamino, C₃₋₆ cycloalkylcarbonylamino, N-(C₁₋₄ alkyl)aminocarbonyl, N,N-di(C₁₋₄ alkyl)aminocarbonyl, C₁₋₄ alkyloxycarbonylamino, C₃₋₆ cycloalkyloxycarbonylamino, C₁₋₄ alkylsulfonamido and C₃₋₆ cycloalkylsulfonamido;
n is an integer 1, 2 or 3;
R³ is selected from the group consisting of hydrogen, halogen, cyano, C₁₋₄ alkyl, C₃₋₆ cycloalkyl, C₁₋₄ alkoxy, C₃₋₆ cycloalkyloxy, C₁₋₄ alkylthio, C₃₋₆ cycloalkylthio, amino, N-(C₁₋₄ alkyl)amino and N,N-di(C₁₋₄ alkyl)amino;
R^{4A} and R^{4B} are each independently selected from the group consisting of hydrogen, halogen, hydroxy, C₁₋₄ alkyl and C₁₋₄ alkoxy; or R^{4A} and R^{4B}, together with the carbon atom to which they are attached, form a 3- to 5-membered saturated carbocyclic ring;
R^{4C} and R^{4D} are each independently selected from the group consisting of hydrogen and C₁₋₄ alkyl; and
R⁵ is selected from the group consisting of hydrogen and C₁₋₄ alkyl; or a pharmaceutically acceptable salt thereof.

In some embodiments, R¹ is C₂₋₄ alkyl. In some embodiments, R¹ is n-propyl. In some embodiments, R¹ is n-butyl.

In some embodiments, R² is selected from the group consisting of hydrogen, fluoro, chloro, bromo, hydroxy, methoxy, amino, methylamino and dimethylamino. In some embodiments, n is 1, i.e. the phenyl-ring is substituted with only one substituent R². In some embodiments, R² is in the para-position.

In some embodiments, R³ is selected from the group consisting of fluoro, chloro, bromo, methyl, cyclopropyl, methoxy, ethoxy, methylthio, ethylthio, amino, methylamino and dimethylamino.

In some embodiments, R^{4A} and R^{4B} are each independently selected from the group consisting of hydrogen, halogen, hydroxy, C₁₋₄ alkyl and C₁₋₄ alkoxy, or R^{4A} and R^{4B}, together with the carbon atom to which they are attached, form a cyclopropyl ring. In some embodiments, R^{4A} and R^{4B} are each independently fluoro, methyl or methoxy, or together with the carbon atom to which they are attached form a cyclopropyl ring.

In some embodiments, R^{4C} and R^{4D} are each independently hydrogen or methyl. In some embodiments, R^{4C} and R^{4D} are each hydrogen.

In some embodiments, R⁵ is hydrogen. In some embodiments, R⁵ is methyl.

In some embodiments, the compound of formula (II) is a compound of formula (II-a):

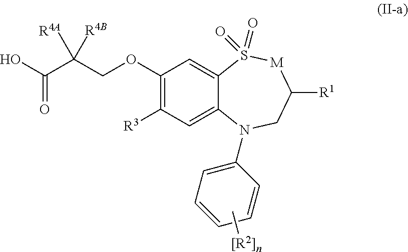

wherein
M is selected from the group consisting of —CH₂—, —NH— and —NCH₃—;
R¹ is C₂₋₄ alkyl;
R² is independently selected from the group consisting of hydrogen, halogen, hydroxy, C₁₋₄ alkyl, C₁₋₄ haloalkyl, C₁₋₄ alkoxy, C₁₋₄ haloalkoxy, amino, N-(C₁₋₄ alkyl)amino, N,N-di(C₁₋₄ alkyl)amino;
n is an integer 1 or 2;
R³ is selected from the group consisting of halogen, C₁₋₄ alkyl, C₃₋₆ cycloalkyl, C₁₋₄ alkoxy, C₁₋₄ alkylthio, amino, N-(C₁₋₄ alkyl)amino and N,N-di(C₁₋₄ alkyl)amino;
R^{4A} and R^{4B} are each independently selected from the group consisting of hydrogen, halogen, hydroxy, C₁₋₄ alkyl and C₁₋₄ alkoxy, or R^{4A} and R^{4B}, together with the carbon atom to which they are attached, form a cyclopropyl ring;
or a pharmaceutically acceptable salt thereof.

In some embodiments, the compound of formula (II) is a compound of formula (II-b):

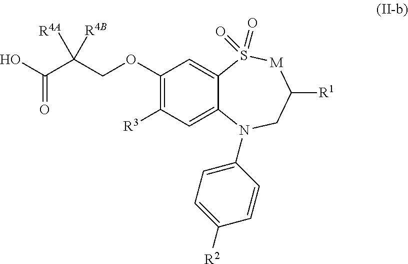

wherein
M is selected from the group consisting of —CH₂—, —NH— and —N(CH₃)—;
R¹ is C₂₋₄ alkyl, more preferably n-propyl or n-butyl;
R² is independently selected from the group consisting of hydrogen, fluoro, chloro, bromo, hydroxy, methoxy, amino, methylamino, dimethylamino;
R³ is selected from the group consisting of fluoro, chloro, bromo, methyl, cyclopropyl, methoxy, ethoxy, methylthio, ethylthio, amino, methylamino and dimethylamino;
R^{4A} and R^{4B} are each independently hydrogen, fluoro, methyl, methoxy or ethoxy, or together with the carbon atom to which they are attached form a cyclopropyl ring;
or a pharmaceutically acceptable salt thereof.

Compounds of formula (II-b), as defined above, also include compounds wherein M, R¹, R², R³, R^{4A} and R^{4B} are as indicated in Table 2 below, or a pharmaceutically acceptable salt thereof:

TABLE 2

| M | R¹ | R² | R³ | R^{4A} | R^{4B} |
|---|---|---|---|---|---|
| CH₂ | CH₂CH₂CH₃ | H | SCH₃ | CH₃ | CH₃ |
| CH₂ | CH₂CH₂CH₃ | H | SCH₃ | —CH₂CH₂— | |
| CH₂ | CH₂CH₂CH₃ | H | SCH₃ | F | F |
| CH₂ | CH₂CH₂CH₃ | F | SCH₃ | CH₃ | CH₃ |
| CH₂ | CH₂CH₂CH₃ | F | SCH₃ | —CH₂CH₂— | |
| CH₂ | CH₂CH₂CH₃ | F | SCH₃ | F | F |
| NH | CH₂CH₂CH₃ | H | SCH₃ | CH₃ | CH₃ |
| NH | CH₂CH₂CH₃ | H | SCH₃ | —CH₂CH₂— | |
| NH | CH₂CH₂CH₃ | H | SCH₃ | F | F |
| NH | CH₂CH₂CH₃ | F | SCH₃ | CH₃ | CH₃ |
| NH | CH₂CH₂CH₃ | F | SCH₃ | —CH₂CH₂— | |
| NH | CH₂CH₂CH₃ | F | SCH₃ | F | F |
| NCH₃ | CH₂CH₂CH₃ | H | SCH₃ | CH₃ | CH₃ |
| NCH₃ | CH₂CH₂CH₃ | H | SCH₃ | —CH₂CH₂— | |
| NCH₃ | CH₂CH₂CH₃ | H | SCH₃ | F | F |
| NCH₃ | CH₂CH₂CH₃ | F | SCH₃ | CH₃ | CH₃ |
| NCH₃ | CH₂CH₂CH₃ | F | SCH₃ | —CH₂CH₂— | |
| NCH₃ | CH₂CH₂CH₃ | F | SCH₃ | F | F |
| CH₂ | CH₂CH₂CH₃ | H | SCH₂CH₃ | CH₃ | CH₃ |
| CH₂ | CH₂CH₂CH₃ | H | SCH₂CH₃ | —CH₂CH₂— | |
| CH₂ | CH₂CH₂CH₃ | H | SCH₂CH₃ | F | F |
| CH₂ | CH₂CH₂CH₃ | F | SCH₂CH₃ | CH₃ | CH₃ |
| CH₂ | CH₂CH₂CH₃ | F | SCH₂CH₃ | —CH₂CH₂— | |
| CH₂ | CH₂CH₂CH₃ | F | SCH₂CH₃ | F | F |
| NH | CH₂CH₂CH₃ | H | SCH₂CH₃ | CH₃ | CH₃ |
| NH | CH₂CH₂CH₃ | H | SCH₂CH₃ | —CH₂CH₂— | |
| NH | CH₂CH₂CH₃ | H | SCH₂CH₃ | F | F |
| NH | CH₂CH₂CH₃ | F | SCH₂CH₃ | CH₃ | CH₃ |
| NH | CH₂CH₂CH₃ | F | SCH₂CH₃ | —CH₂CH₂— | |
| NH | CH₂CH₂CH₃ | F | SCH₂CH₃ | F | F |
| NCH₃ | CH₂CH₂CH₃ | H | SCH₂CH₃ | CH₃ | CH₃ |
| NCH₃ | CH₂CH₂CH₃ | H | SCH₂CH₃ | —CH₂CH₂— | |
| NCH₃ | CH₂CH₂CH₃ | H | SCH₂CH₃ | F | F |
| NCH₃ | CH₂CH₂CH₃ | F | SCH₂CH₃ | CH₃ | CH₃ |
| NCH₃ | CH₂CH₂CH₃ | F | SCH₂CH₃ | —CH₂CH₂— | |
| NCH₃ | CH₂CH₂CH₃ | F | SCH₂CH₃ | F | F |
| CH₂ | CH₂CH₂CH₃ | H | N(CH₃)₂ | CH₃ | CH₃ |
| CH₂ | CH₂CH₂CH₃ | H | N(CH₃)₂ | —CH₂CH₂— | |
| CH₂ | CH₂CH₂CH₃ | H | N(CH₃)₂ | F | F |
| CH₂ | CH₂CH₂CH₃ | F | N(CH₃)₂ | CH₃ | CH₃ |
| CH₂ | CH₂CH₂CH₃ | F | N(CH₃)₂ | —CH₂CH₂— | |
| CH₂ | CH₂CH₂CH₃ | F | N(CH₃)₂ | F | F |
| NH | CH₂CH₂CH₃ | H | N(CH₃)₂ | CH₃ | CH₃ |
| NH | CH₂CH₂CH₃ | H | N(CH₃)₂ | —CH₂CH₂— | |
| NH | CH₂CH₂CH₃ | H | N(CH₃)₂ | F | F |
| NH | CH₂CH₂CH₃ | F | N(CH₃)₂ | CH₃ | CH₃ |
| NH | CH₂CH₂CH₃ | F | N(CH₃)₂ | —CH₂CH₂— | |
| NH | CH₂CH₂CH₃ | F | N(CH₃)₂ | F | F |
| NCH₃ | CH₂CH₂CH₃ | H | N(CH₃)₂ | CH₃ | CH₃ |
| NCH₃ | CH₂CH₂CH₃ | H | N(CH₃)₂ | —CH₂CH₂— | |
| NCH₃ | CH₂CH₂CH₃ | H | N(CH₃)₂ | F | F |
| NCH₃ | CH₂CH₂CH₃ | F | N(CH₃)₂ | CH₃ | CH₃ |
| NCH₃ | CH₂CH₂CH₃ | F | N(CH₃)₂ | —CH₂CH₂— | |
| NCH₃ | CH₂CH₂CH₃ | F | N(CH₃)₂ | F | F |
| CH₂ | CH₂CH₂CH₂CH₃ | H | SCH₃ | CH₃ | CH₃ |
| CH₂ | CH₂CH₂CH₂CH₃ | H | SCH₃ | —CH₂CH₂— | |
| CH₂ | CH₂CH₂CH₂CH₃ | H | SCH₃ | F | F |
| CH₂ | CH₂CH₂CH₂CH₃ | F | SCH₃ | CH₃ | CH₃ |
| CH₂ | CH₂CH₂CH₂CH₃ | F | SCH₃ | —CH₂CH₂— | |
| CH₂ | CH₂CH₂CH₂CH₃ | F | SCH₃ | F | F |
| NH | CH₂CH₂CH₂CH₃ | H | SCH₃ | CH₃ | CH₃ |
| NH | CH₂CH₂CH₂CH₃ | H | SCH₃ | —CH₂CH₂— | |
| NH | CH₂CH₂CH₂CH₃ | H | SCH₃ | F | F |
| NH | CH₂CH₂CH₂CH₃ | F | SCH₃ | CH₃ | CH₃ |
| NH | CH₂CH₂CH₂CH₃ | F | SCH₃ | —CH₂CH₂— | |
| NH | CH₂CH₂CH₂CH₃ | F | SCH₃ | F | F |
| NCH₃ | CH₂CH₂CH₂CH₃ | H | SCH₃ | CH₃ | CH₃ |
| NCH₃ | CH₂CH₂CH₂CH₃ | H | SCH₃ | —CH₂CH₂— | |
| NCH₃ | CH₂CH₂CH₂CH₃ | H | SCH₃ | F | F |
| NCH₃ | CH₂CH₂CH₂CH₃ | F | SCH₃ | CH₃ | CH₃ |
| NCH₃ | CH₂CH₂CH₂CH₃ | F | SCH₃ | —CH₂CH₂— | |
| NCH₃ | CH₂CH₂CH₂CH₃ | F | SCH₃ | F | F |
| CH₂ | CH₂CH₂CH₂CH₃ | H | SCH₂CH₃ | CH₃ | CH₃ |
| CH₂ | CH₂CH₂CH₂CH₃ | H | SCH₂CH₃ | —CH₂CH₂— | |
| CH₂ | CH₂CH₂CH₂CH₃ | H | SCH₂CH₃ | F | F |
| CH₂ | CH₂CH₂CH₂CH₃ | F | SCH₂CH₃ | CH₃ | CH₃ |
| CH₂ | CH₂CH₂CH₂CH₃ | F | SCH₂CH₃ | —CH₂CH₂— | |
| CH₂ | CH₂CH₂CH₂CH₃ | F | SCH₂CH₃ | F | F |
| NH | CH₂CH₂CH₂CH₃ | H | SCH₂CH₃ | CH₃ | CH₃ |
| NH | CH₂CH₂CH₂CH₃ | H | SCH₂CH₃ | —CH₂CH₂— | |
| NH | CH₂CH₂CH₂CH₃ | H | SCH₂CH₃ | F | F |
| NH | CH₂CH₂CH₂CH₃ | F | SCH₂CH₃ | CH₃ | CH₃ |
| NH | CH₂CH₂CH₂CH₃ | F | SCH₂CH₃ | —CH₂CH₂— | |
| NH | CH₂CH₂CH₂CH₃ | F | SCH₂CH₃ | F | F |
| NCH₃ | CH₂CH₂CH₂CH₃ | H | SCH₂CH₃ | CH₃ | CH₃ |
| NCH₃ | CH₂CH₂CH₂CH₃ | H | SCH₂CH₃ | —CH₂CH₂— | |
| NCH₃ | CH₂CH₂CH₂CH₃ | H | SCH₂CH₃ | F | F |
| NCH₃ | CH₂CH₂CH₂CH₃ | F | SCH₂CH₃ | CH₃ | CH₃ |
| NCH₃ | CH₂CH₂CH₂CH₃ | F | SCH₂CH₃ | —CH₂CH₂— | |
| NCH₃ | CH₂CH₂CH₂CH₃ | F | SCH₂CH₃ | F | F |
| CH₂ | CH₂CH₂CH₂CH₃ | H | N(CH₃)₂ | CH₃ | CH₃ |
| CH₂ | CH₂CH₂CH₂CH₃ | H | N(CH₃)₂ | —CH₂CH₂— | |
| CH₂ | CH₂CH₂CH₂CH₃ | H | N(CH₃)₂ | F | F |
| CH₂ | CH₂CH₂CH₂CH₃ | F | N(CH₃)₂ | CH₃ | CH₃ |
| CH₂ | CH₂CH₂CH₂CH₃ | F | N(CH₃)₂ | —CH₂CH₂— | |
| CH₂ | CH₂CH₂CH₂CH₃ | F | N(CH₃)₂ | F | F |
| NH | CH₂CH₂CH₂CH₃ | H | N(CH₃)₂ | CH₃ | CH₃ |
| NH | CH₂CH₂CH₂CH₃ | H | N(CH₃)₂ | —CH₂CH₂— | |
| NH | CH₂CH₂CH₂CH₃ | H | N(CH₃)₂ | F | F |
| NH | CH₂CH₂CH₂CH₃ | F | N(CH₃)₂ | CH₃ | CH₃ |
| NH | CH₂CH₂CH₂CH₃ | F | N(CH₃)₂ | —CH₂CH₂— | |
| NH | CH₂CH₂CH₂CH₃ | F | N(CH₃)₂ | F | F |
| NCH₃ | CH₂CH₂CH₂CH₃ | H | N(CH₃)₂ | CH₃ | CH₃ |
| NCH₃ | CH₂CH₂CH₂CH₃ | H | N(CH₃)₂ | —CH₂CH₂— | |
| NCH₃ | CH₂CH₂CH₂CH₃ | H | N(CH₃)₂ | F | F |
| NCH₃ | CH₂CH₂CH₂CH₃ | F | N(CH₃)₂ | CH₃ | CH₃ |
| NCH₃ | CH₂CH₂CH₂CH₃ | F | N(CH₃)₂ | —CH₂CH₂— | |
| NCH₃ | CH₂CH₂CH₂CH₃ | F | N(CH₃)₂ | F | F |

In some embodiments, the compound of formula (II) is selected from the group consisting of:
3-((3-Butyl-2-methyl-7-(methylthio)-1,1-dioxido-5-phenyl-2,3,4,5-tetrahydro-1,2,5-benzothiadiazepin-8-yl)oxy)-2,2-dimethylpropanoic acid;
(S)-3-((3-Butyl-2-methyl-7-(methylthio)-1,1-dioxido-5-phenyl-2,3,4,5-tetrahydro-1,2,5-benzothiadiazepin-8-yl)oxy)-2,2-dimethylpropanoic acid;
(R)-3-((3-butyl-2-methyl-7-(methylthio)-1,1-dioxido-5-phenyl-2,3,4,5-tetrahydro-1,2,5-benzothiadiazepin-8-yl)oxy)-2,2-dimethylpropanoic acid;
1-(((3-Butyl-2-methyl-7-(methylthio)-1,1-dioxido-5-phenyl-2,3,4,5-tetrahydro-1,2,5-benzothiadiazepin-8-yl)oxy)methyl)cyclopropane-1-carboxylic acid;
(S)-1-(((3-Butyl-2-methyl-7-(methylthio)-1,1-dioxido-5-phenyl-2,3,4,5-tetrahydro-1,2,5-benzothiadiazepin-8-yl)oxy)methyl)cyclopropane-1-carboxylic acid;
(R)-1-(((3-butyl-2-methyl-7-(methylthio)-1,1-dioxido-5-phenyl-2,3,4,5-tetrahydro-1,2,5-benzothiadiazepin-8-yl)oxy)methyl)cyclopropane-1-carboxylic acid;
3-((3-Butyl-7-(ethylthio)-2-methyl-1,1-dioxido-5-phenyl-2,3,4,5-tetrahydro-1,2,5-benzothiadiazepin-8-yl)oxy)-2,2-dimethylpropanoic acid;
(S)-3-((3-Butyl-7-(ethylthio)-2-methyl-1,1-dioxido-5-phenyl-2,3,4,5-tetrahydro-1,2,5-benzothiadiazepin-8-yl)oxy)-2,2-dimethylpropanoic acid;
(R)-3-((3-butyl-7-(ethylthio)-2-methyl-1,1-dioxido-5-phenyl-2,3,4,5-tetrahydro-1,2,5-benzothiadiazepin-8-yl)oxy)-2,2-dimethylpropanoic acid;
1-(((3-Butyl-7-(ethylthio)-2-methyl-1,1-dioxido-5-phenyl-2,3,4,5-tetrahydro-1,2,5-benzothiadiazepin-8-yl)oxy)methyl)cyclopropane-1-carboxylic acid;
(S)-1-(((3-butyl-7-(ethylthio)-2-methyl-1,1-dioxido-5-phenyl-2,3,4,5-tetrahydro-1,2,5-benzothiadiazepin-8-yl)oxy)methyl)cyclopropane-1-carboxylic acid;
(R)-1-(((3-butyl-7-(ethylthio)-2-methyl-1,1-dioxido-5-phenyl-2,3,4,5-tetrahydro-1,2,5-benzothiadiazepin-8-yl)oxy)methyl)cyclopropane-1-carboxylic acid;
3-((3-butyl-5-(4-fluorophenyl)-7-(methylthio)-1,1-dioxido-2,3,4,5-tetrahydro-1,5-benzothiazepin-8-yl)oxy)-2,2-dimethylpropanoic acid;
1-(((3-butyl-5-(4-fluorophenyl)-7-(methylthio)-1,1-dioxido-2,3,4,5-tetrahydro-1,5-benzothiazepin-8-yl)oxy)methyl)cyclopropane-1-carboxylic acid;
1-(((3-butyl-5-(4-fluorophenyl)-2-methyl-7-(methylthio)-1,1-dioxido-2,3,4,5-tetrahydro-1,2,5-benzothiadiazepin-8-yl)oxy)methyl)cyclopropane-1-carboxylic acid;
(S)-1-(((3-butyl-5-(4-fluorophenyl)-2-methyl-7-(methylthio)-1,1-dioxido-2,3,4,5-tetrahydro-1,2,5-benzothiadiazepin-8-yl)oxy)methyl)cyclopropane-1-carboxylic acid;
(R)-1-(((3-butyl-5-(4-fluorophenyl)-2-methyl-7-(methylthio)-1,1-dioxido-2,3,4,5-tetrahydro-1,2,5-benzothiadiazepin-8-yl)oxy)methyl)cyclopropane-1-carboxylic acid;
3-((3-butyl-5-(4-fluorophenyl)-2-methyl-7-(methylthio)-1,1-dioxido-2,3,4,5 -tetrahydro-1,2,5-benzothiadiazepin-8-yl)oxy)-2,2-dimethylpropanoic acid;
(S)-3-((3-butyl-5-(4-fluorophenyl)-2-methyl-7-(methylthio)-1,1-dioxido-2,3,4,5-tetrahydro-1,2,5-benzothiadiazepin-8-yl)oxy)-2,2-dimethylpropanoic acid;
(R)-3-((3-butyl-5-(4-fluorophenyl)-2-methyl-7-(methylthio)-1,1-dioxido-2,3,4,5-tetrahydro-1,2,5-benzothiadiazepin-8-yl)oxy)-2,2-dimethylpropanoic acid;
1-(((3-butyl-7-(ethylthio)-1,1-dioxido-5-phenyl-2,3,4,5-tetrahydro-1,2,5-benzothiadiazepin-8-yl)oxy)methyl)cyclopropane-1-carboxylic acid;
1-(((3-butyl-5-(4-fluorophenyl)-7-(methylthio)-1,1-dioxido-2,3,4,5-tetrahydro-1,2,5-benzothiadiazepin-8-yl)oxy)methyl)cyclopropane-1-carboxylic acid;
(S)-1-(((3-butyl-5-(4-fluorophenyl)-7-(methylthio)-1,1-dioxido-2,3,4,5-tetrahydro-1,2,5 -benzothiadiazepin-8-yl)oxy)methyl)cyclopropane-1-carboxylic acid;
(R)-1-(((3-butyl-5-(4-fluorophenyl)-7-(methylthio)-1,1-dioxido-2,3,4,5-tetrahydro-1,2,5-benzothiadiazepin-8-yl)oxy)methyl)cyclopropane-1-carboxylic acid;
1-(((3-butyl-7-(ethylthio)-5-(4-fluorophenyl)-1,1-dioxido-2,3,4,5-tetrahydro-1,2,5-benzothiadiazepin-8-yl)oxy)methyl)cyclopropane-1-carboxylic acid;
(S)-1-(((3-butyl-7-(ethylthio)-5-(4-fluorophenyl)-1,1-dioxido-2,3,4,5-tetrahydro-1,2,5-benzothiadiazepin-8-yl)oxy)methyl)cyclopropane-1-carboxylic acid;
(R)-1-(((3-butyl-7-(ethylthio)-5-(4-fluorophenyl)-1,1-dioxido-2,3,4,5-tetrahydro-1,2,5-benzothiadiazepin-8-yl)oxy)methyl)cyclopropane-1-carboxylic acid;
3-((3-butyl-2-methyl-7-(methylthio)-1,1-dioxido-5-phenyl-2,3,4,5-tetrahydro-1,2,5-benzothiadiazepin-8-yl)oxy)-2,2-difluoropropanoic acid;
(S)-3-((3-butyl-2-methyl-7-(methylthio)-1,1-dioxido-5-phenyl-2,3,4,5-tetrahydro-1,2,5-benzothiadiazepin-8-yl)oxy)-2,2-difluoropropanoic acid;
(R)-3-((3-butyl-2-methyl-7-(methylthio)-1,1-dioxido-5-phenyl-2,3,4,5-tetrahydro-1,2,5-benzothiadiazepin-8-yl)oxy)-2,2-difluoropropanoic acid;
3-((3-Butyl-2-methyl-7-(methylthio)-1,1-dioxido-5-phenyl-2,3,4,5-tetrahydro-1,2,5-benzothiadiazepin-8-yl)oxy)-2-methoxy-2-methylpropanoic acid;
(S)-3-(((R)-3-Butyl-2-methyl-7-(methylthio)-1,1-dioxido-5-phenyl-2,3,4,5-tetrahydro-1,2,5-benzothiadiazepin-8-yl)oxy)-2-methoxy-2-methylpropanoic acid;
(S)-3-(((S)-3-Butyl-2-methyl-7-(methylthio)-1,1-dioxido-5-phenyl-2,3,4,5-tetrahydro-1,2,5-benzothiadiazepin-8-yl)oxy)-2-methoxy-2-methylpropanoic acid;
(R)-3-(((R)-3-butyl-2-methyl-7-(methylthio)-1,1-dioxido-5-phenyl-2,3,4,5-tetrahydro-1,2,5-benzothiadiazepin-8-yl)oxy)-2-methoxy-2-methylpropanoic acid;
(R)-3-(((S)-3-butyl-2-methyl-7-(methylthio)-1,1-dioxido-5-phenyl-2,3,4,5-tetrahydro-1,2,5-benzothiadiazepin-8-yl)oxy)-2-methoxy-2-methylpropanoic acid;
3-((3-butyl-7-(methylthio)-1,1-dioxido-5-phenyl-2,3,4,5-tetrahydro-1,5-benzothiazepin-8-yl)oxy)propanoic acid;
(S)-3-((3-butyl-7-(methylthio)-1,1-dioxido-5-phenyl-2,3,4,5-tetrahydro-1,5-benzothiazepin-8-yl)oxy)propanoic acid;
(R)-3-((3-butyl-7-(methylthio)-1,1-dioxido-5-phenyl-2,3,4,5-tetrahydro-1,5-benzothiazepin-8-yl)oxy)propanoic acid;
3-((3-butyl-2-methyl-7-(methylthio)-1,1-dioxido-5-phenyl-2,3,4,5-tetrahydro-1,2,5-benzothiadiazepin-8-yl)oxy)-2-ethoxypropanoic acid;
3-((3-butyl-7-(methylthio)- 1,1-dioxido-5-phenyl-2,3,4,5-tetrahydro-1,5-benzothiazepin-8-yl)oxy)-2-hydroxypropanoic acid;
3-((3-ethyl-7-(methylthio)-1,1-dioxido-5-phenyl-2,3,4,5-tetrahydro-1,5-benzothiazepin-8-yl)oxy)propanoic acid;
3-((3-butyl-2-methyl-7-(methylthio)-1,1-dioxido-5-phenyl-2,3,4,5-tetrahydro-1,2,5-benzothiadiazepin-8-yl)oxy)-2-methoxypropanoic acid;
3-(((S)-3-butyl-2-methyl-7-(methylthio)-1,1-dioxido-5-phenyl-2,3,4,5-tetrahydro-1,2,5-benzothiadiazepin-8-yl)oxy)-2-methoxypropanoic acid;
3-(((R)-3-butyl-2-methyl-7-(methylthio)-1,1-dioxido-5-phenyl-2,3,4,5-tetrahydro-1,2,5-benzothiadiazepin-8-yl)oxy)-2-methoxypropanoic acid;
(S)-3-(((R)-3-butyl-2-methyl-7-(methylthio)-1,1-dioxido-5-phenyl-2,3,4,5-tetrahydro-1,2,5-benzothiadiazepin-8-yl)oxy)-2-methoxypropanoic acid;
(R)-3-(((R)-3-butyl-2-methyl-7-(methylthio)-1,1-dioxido-5-phenyl-2,3,4,5-tetrahydro-1,2,5-benzothiadiazepin-8-yl)oxy)-2-methoxypropanoic acid;
(S)-3-(((S)-3-butyl-2-methyl-7-(methylthio)-1,1-dioxido-5-phenyl-2,3,4,5-tetrahydro-1,2,5-benzothiadiazepin-8-yl)oxy)-2-methoxypropanoic acid;
(R)-3-(((S)-3-butyl-2-methyl-7-(methylthio)-1,1-dioxido-5-phenyl-2,3,4,5-tetrahydro-1,2,5-benzothiadiazepin-8-yl)oxy)-2-methoxypropanoic acid; and
3-((3-butyl-7-(ethylthio)-5-(4-fluorophenyl)-2-methyl-1,1-dioxido-2,3,4,5-tetrahydro-1,2,5-benzothiadiazepin-8-yl)oxy)-2-hydroxypropanoic acid;
or a pharmaceutically acceptable salt thereof.

In some embodiments, the compound of formula (II) is 3-((3-butyl-5-(4-fluorophenyl)-2-methyl-7-(methylthio)-1,1-dioxido-2,3,4,5-tetrahydro-1,2,5-benzothiadiazepin-8-yl)oxy)-2,2-dimethylpropanoic acid:

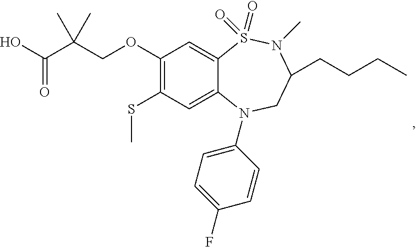 [SMILES: CCCCC1CN(c2ccc(F)cc2)c2cc(SC)c(OCC(C)(C)C(=O)O)cc2S(=O)(=O)N1C]

or a pharmaceutically acceptable salt thereof (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid is also referred to herein as “Compound 2.”

In some embodiments, the compound of formula (II) is (S)-3-((3-butyl-5-(4-fluorophenyl)-2-methyl-7-(methylthio)-1,1-dioxido-2,3,4,5-tetrahydro-1,2,5-benzothiadiazepin-8-yl)oxy)-2,2-dimethylpropanoic acid, or a pharmaceutically acceptable salt thereof, is administered to the subject. In some embodiments, Compound 2 is ((S)-3-((3-butyl-5-(4-fluorophenyl)-2-methyl-7-(methylthio)-1,1-dioxido-2,3,4,5-tetrahydro-1,2,5-benzothiadiazepin-8-yl)oxy)-2,2-dimethylpropanoic acid.

In some embodiments, the compound of formula (II) is (R)-3-((3-butyl-5-(4-fluorophenyl)-2-methyl-7-(methylthio)-1,1-dioxido-2,3,4,5-tetrahydro-1,2,5-benzothiadiazepin-8-yl)oxy)-2,2-dimethylpropanoic acid, or a pharmaceutically acceptable salt thereof, is administered to the subject. In some embodiments, Compound 2 is (R)-3-((3-butyl-5-(4-fluorophenyl)-2-methyl-7-(methylthio)-1,1-dioxido-2,3,4,5-tetrahydro-1,2,5-benzothiadiazepin-8-yl)oxy)-2,2-dimethylpropanoic acid.

Compounds of formula (II) can be prepared as described in U.S. application Ser. No. 17/813,152.

An NTCP inhibitor as used herein can also include compounds disclosed in; International Publication Nos. WO 93/16055, WO 94/18183, WO 94/18184, WO 96/05188, WO 96/08484, WO 96/16051, WO 97/33882, WO 98/07449, WO 98/40375, WO 99/35135, WO 99/64409, WO 99/64410, WO 00/47568, WO 00/61568, WO 00/38725, WO 00/38726, WO 00/38727, WO 00/38728, WO 00/38729, WO 01/66533, WO 01/68096, WO 02/32428, WO 02/50051, WO 03/020710, WO 03/022286, WO 03/022825, WO 03/022830, WO 03/061663, WO 03/091232, WO 2004/006899, WO 2004/076430, WO 2007/009655, WO 2007/009656, WO 2008/058628, WO 2008/058630, WO 2011/137135, WO 2019/234077, WO 2020/161216, WO 2020/161217, WO 2021/110883, WO 2021/110884, WO 2021/110885, WO 2021/110886, WO 2021/110887, WO 2022/029101, WO 2022/253997, and WO 2022/117778; DE 19825804; EP 864582, EP 489423, EP 549967, EP 573848, EP 624593, EP 624594, EP 624595, EP 624596, EP 0864582, EP 1173205, EP 1535913 EP 3210977.

In some embodiments, the subject has hepatitis B. In some embodiments, the subject has acute hepatitis B. For example, a subject that has acute hepatitis B includes a subject that has had hepatitis B for less than 6 months. In some embodiments, the subject has chronic hepatitis B. For example, a subject that has chronic hepatitis B includes a subject that has had hepatitis B for six or more months.

In some embodiments, the subject has hepatitis D. In some embodiments, the subject has acute hepatitis D. For example, a subject that has acute hepatitis D includes a subject that has had hepatitis D for less than 6 months. In some embodiments, the subject has chronic hepatitis D. For example, a subject that has chronic hepatitis D includes a subject that has had hepatitis D for six or more months.

In some embodiments, the subject has hepatitis B and hepatitis D. In some embodiments, the subject has chronic hepatitis B and hepatitis D. In some embodiments, the subject has chronic hepatitis B and acute hepatitis D. In some embodiments, the subject has chronic hepatitis B and chronic hepatitis D.

HDV depends on HBV to replicate and consequently only propagates when coinfecting with HBV. Lempp F A, Urban S. Viruses. 2017;9:172. Co-infection with HDV and HBV is considered the most severe form of chronic viral hepatitis as it can lead to more rapid progression towards hepatocellular carcinoma and liver-related death. World Health Organization Hepatitis D fact sheet, 2021, available at: who.int/news-room/fact-sheets/detail/hepatitis-d. Accordingly, also provided herein are methods for preventing hepatitis D infection in a subject having hepatitis B, comprising administering to the subject one or more NTCP inhibitors (e.g., any of the NTCP inhibitors described herein).

In some of any of the above embodiments, the subject is administered an NTCP inhibitor (e.g., a compound of formula (I), or a pharmaceutically acceptable salt thereof, or a compound of formula (II), or a pharmaceutically acceptable salt thereof) after exposure to hepatitis B and/or hepatitis D. For example, the NTCP inhibitor (e.g., a compound of formula (I), or a pharmaceutically acceptable salt thereof, or a compound of formula (II), or a pharmaceutically acceptable salt thereof) can be administered within about 1 to about 30 hours of exposure to hepatitis B and/or hepatitis D. In some embodiments, the NTCP inhibitor (e.g., a compound of formula (I), or a pharmaceutically acceptable salt thereof, or a compound of formula (II), or a pharmaceutically acceptable salt thereof) can be administered within about 1 to about 2, about 1 to about 5, about 1 to about 10, about 1 to about 15, about 1 to about 20, about 1 to about 25, about 5 to about 10, about 5 to about 15, about 5 to about 20, about 5 to about 25, about 5 to about 30, about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 hours of exposure to hepatitis B and/or D. In some embodiments, the subject is administered an NTCP inhibitor (e.g., a compound of formula (I), or a pharmaceutically acceptable salt thereof, or a compound of formula (II), or a pharmaceutically acceptable salt thereof) within about 18 hours of exposure to hepatitis B and/or hepatitis D.

In some embodiments of any of the methods described herein, the presence of hepatitis B and/or D in a subject can be determined by one or more biomarkers. Non-limiting examples of such biomarkers for hepatitis B include the concentration of HBV DNA, the concentration of hepatitis B surface antigen (HBsAg), the concentration of hepatitis B core antigen (HBcAg), and the concentration of hepatitis B e antigen (HBeAg). Non-limiting examples of such biomarkers for hepatitis D include the concentration of HDV DNA and the concentration of hepatitis D antigen (HDAg). See, e.g., Coffin et al. New and Old Biomarkers for Diagnosis and Management of Chronic Hepatitis B Virus Infection. Gastroenterology. 2019 Jan;156(2):355-368.e3. Such biomarkers can be detected in a sample from the subject (e.g., a serum or biopsy sample).

In some embodiments of any of the methods described herein, an assay used to determine whether the subject has hepatitis B and/or hepatitis D, using a sample from a subject can include, but is not limited to, next generation sequencing, immunohistochemistry, Southern blotting, Western blotting, Northern blotting, and PCR-based amplification (e.g., RT-PCR and quantitative real-time RT-PCR). As is well-known in the art, hepatitis antigens such as HBsAg, HBcAg, HBeAg, and HDAg may be detected using an immunoassay. Non-limiting examples of such assays include enzyme-linked immunosorbent assays (ELISA), radioimmunoassay (RIA), enzyme immunoassay (EIA), electrochemiluminescence immunoassay (ECLIA), microparticle enzyme immunoassay (MEIA), and chemiluminescent microparticle immunoassay (CMIA). See, e.g., Tyas et al. Recent Advances of Hepatitis B Detection towards Paper-Based Analytical Devices. ScientificWorldJournal. 2021 Feb 26;2021:6643573. HBV DNA and/or HDV DNA can be detected using, for example, PCR-based amplification (e.g., RT-PCR and quantitative real-time RT-PCR). See, e.g., Coffin et al. Gastroenterology. 2019 Jan;156(2):355-368.e3.

In some embodiments, a biomarker as described herein (e.g., HBV DNA, HBsAg, HBcAg, HBeAg, HDV DNA, or HDAg) can be used to monitor the responsiveness of a subject to a particular therapy (e.g., an NTCP inhibitor such as a compound of formula (I), or a pharmaceutically acceptable salt thereof, or a compound of formula (II), or a pharmaceutically acceptable salt thereof). For example, prior to starting treatment with an NTCP inhibitor as described herein (e.g., a compound of formula (I), or a pharmaceutically acceptable salt thereof, or a compound of formula (II), or a pharmaceutically acceptable salt thereof), a sample can be obtained from the subject and the level of the biomarker determined in the sample. This sample can be considered a reference sample. The subject can then be administered one or more doses of an NTCP inhibitor as described herein (e.g., a compound of formula (I), or a pharmaceutically acceptable salt thereof, or a compound of formula (II), or a pharmaceutically acceptable salt thereof) and the levels of the biomarker can be monitored after administration. For example, the levels of the biomarker can be monitored after the first dose, second dose, third dose, etc. or after one week, two weeks, three weeks, four weeks, etc. If the concentration of the biomarker is decreased (i.e., reduced) relative to the reference sample, this is indicative of responsiveness to the therapy. In some embodiments, the level of the biomarker is decreased relative to the reference sample by about 1% to about 99%, about 1% to about 95%, about 1% to about 90%, about 1% to about 85%, about 1% to about 80%, about 1% to about 75%, about 1% to about 70%, about 1% to about 65%, about 1% to about 60%, about 1% to about 55%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 99%, about 10% to about 99%, about 15% to about 99%, about 20% to about 99%, about 25% to about 99%, about 30% to about 99%, about 35% to about 99%, about 40% to about 99%, about 45% to about 99%, about 50% to about 99%, about 55% to about 99%, about 60% to about 99%, about 65% to about 99%, about 70% to about 99%, about 75% to about 95%, about 80% to about 99%, about 90% to about 99%, about 95% to about 99%, about 5% to about 10%, about 5% to about 25%, about 10% to about 30%, about 20% to about 40%, about 25% to about 50%, about 35% to about 55%, about 40% to about 60%, about 50% to about 75%, about 60% to about 80%, about 65% to about 85%, about 70% to about 90%, or about 75% to about 95%. In some embodiments, the concentration of the biomarker is reduced such that it is below the detection limit of the instrument, i.e., the level of the biomarker is “undetectable.”

In some embodiments, the severity of hepatitis B and/or D is determined by one or more biomarkers indicative of one or more of liver damage, inflammation, liver fibrosis, and/or liver cirrhosis or scoring systems thereof. The concentration of such biomarkers can be determined by, for example, measuring, quantifying, and monitoring the expression level of the gene or mRNA encoding the biomarker and/or the peptide or protein of the biomarker. Non-limiting examples of biomarkers indicative of one or more of liver damage, inflammation, liver fibrosis, and/or liver cirrhosis and/or scoring systems thereof include the aspartate aminotransferase (AST) to platelet ratio index (APRI); the aspartate aminotransferase (AST) and alanine aminotransferase (ALT) ratio (AAR); the FIB-4 score, which is based on the APRI, alanine aminotransferase (ALT) levels, and age of the subject (see, e.g., McPherson et al., Gut 2010, vol. 59(9), p. 1265-9); hyaluronic acid; pro-inflammatory cytokines; a panel of biomarkers consisting of α2-macroglobulin, haptoglobin, apolipoprotein A1, bilirubin, gamma glutamyl transpeptidase (GGT) combined with a subject's age and gender to generate a measure of fibrosis and necroinflammatory activity in the liver (e.g., FIBROTEST®, FIBROSURE®), a panel of biomarkers consisting of bilirubin, gamma-glutamyltransferase, hyaluronic acid, α2-macroglobulin combined with the subject's age and sex (e.g., HEPASCORE®; see, e.g., Adams et al., Clin. Chem. 2005, vol. 51(10), p. 1867-1873), and a panel of biomarkers consisting of tissue inhibitor of metalloproteinase-1, hyaluronic acid, and α2-macroglobulin (e.g., FIBROSPECT®); a panel of biomarkers consisting of tissue inhibitor of metalloproteinases 1 (TIMP-1), amino-terminal propeptide of type III procollagen (PIIINP) and hyaluronic acid (HA) (e.g., the Enhanced Liver Fibrosis (ELF) score, see, e.g., Lichtinghagen R, et al., J Hepatol. 2013 Aug;59(2):236-42). In some embodiments, the presence of fibrosis in a subject having hepatitis B and/or hepatitis D is determined by one or more of the FIB-4 score, a panel of biomarkers consisting of α2-macroglobulin, haptoglobin, apolipoprotein A1, bilirubin, gamma glutamyl transpeptidase (GGT) combined with a subject's age and gender to generate a measure of fibrosis and necroinflammatory activity in the liver (e.g., FIBROTEST®, FIBROSURE®), a panel of biomarkers consisting of bilirubin, gamma-glutamyltransferase, hyaluronic acid, α2-macroglobulin combined with the subject's age and sex (e.g., HEPASCORE®; see, e.g., Adams et al., Clin. Chem. 2005, vol. 51(10), p. 1867-1873), and a panel of biomarkers consisting of tissue inhibitor of metalloproteinase-1, hyaluronic acid, and α2-macroglobulin (e.g., FIBROSPECT®); and a panel of biomarkers consisting of tissue inhibitor of metalloproteinases 1 (TIMP-1), amino-terminal propeptide of type III procollagen (PIIINP) and hyaluronic acid (HA) (e.g., the Enhanced Liver Fibrosis (ELF) score).

In some embodiments, the concentration of aspartate aminotransferase (AST) in a sample from the subject decreases or does not increase after administration of an NTCP inhibitor as described herein (e.g., a compound of formula (I), or a pharmaceutically acceptable salt thereof, or a compound of formula (II), or a pharmaceutically acceptable salt thereof). In some embodiments, the concentration of alanine aminotransferase (ALT) in a sample from the subject decreases or does not increase after administration of an NTCP inhibitor as described herein (e.g., a compound of formula (I), or a pharmaceutically acceptable salt thereof, or a compound of formula (II), or a pharmaceutically acceptable salt thereof).

In some embodiments, a decrease in the concentration of one or more biomarkers indicative of one or more of liver damage, inflammation, liver fibrosis, and/or liver cirrhosis during the period of time or after the period of time of administration of an NTCP inhibitor as described herein (e.g., a compound of formula (I), or a pharmaceutically acceptable salt thereof, or a compound of formula (II), or a pharmaceutically acceptable salt thereof) compared to prior to administration of the NTCP inhibitor indicates treatment of hepatitis B and/or D. For example, a decrease in the concentration of one or more biomarkers indicative of one or more of liver damage, inflammation, liver fibrosis, and/or liver cirrhosis by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% indicates treatment of hepatitis B and/or D. In some embodiments, the decrease in the concentration in the level of one or more biomarkers indicative of one or more of liver damage, inflammation, liver fibrosis, and/or liver cirrhosis following administration of the NTCP inhibitor is by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%.

In some embodiments, the “concentration” of an enzyme or antigen refers to the concentration of the enzyme within, e.g., blood or serum. For example, the level of AST or ALT can be expressed as Units/L.

Samples can be obtained from a subject at multiple times during a course of diagnosis, a course of monitoring, and/or a course of treatment (e.g., with an NTCP inhibitor as described herein) to determine one or more clinically relevant parameters including, without limitation, progression of the hepatitis B and/or hepatitis D and efficacy of a treatment. For example, a first sample can be obtained at a first time point and a second sample can be obtained at a second time point during a course of diagnosis, a course of monitoring, and/or a course of treatment. In some embodiments, the first time point can be a time point prior to diagnosing a subject with hepatitis B and/or hepatitis D (e.g., when the subject is healthy), and the second time point can be a time point after subject has developed hepatitis B and/or hepatitis D (e.g., the second time point can be used to diagnose the subject with the disease). In some embodiments, the first time point can be a time point prior to diagnosing a subject with a hepatitis B and/or hepatitis D (e.g., when the subject is healthy), after which the subject is monitored, and the second time point can be a time point after monitoring the subject. In some embodiments, the first time point can be a time point after diagnosing a subject with hepatitis B and/or hepatitis D, after which an NTCP inhibitor (e.g., a compound of formula (I), or a pharmaceutically acceptable salt thereof, or a compound of formula (II), or a pharmaceutically acceptable salt thereof) is administered to the subject, and the second time point can be a time point after the NTCP inhibitor (e.g., a compound of formula (I), or a pharmaceutically acceptable salt thereof, or a compound of formula (II), or a pharmaceutically acceptable salt thereof) is administered; in such cases, the second time point can be used to assess the efficacy of the NTCP inhibitor (e.g., if the concentrations of one or more biomarkers detected at the second time point are reduced or are undetectable compared to the first time point).

In some embodiments, the time difference between the first and second time points is about 1 day to about 1 year, about 1 day to about 11 months, about 1 day to about 10 months, about 1 day to about 9 months, about 1 day to about 8 months, about 1 day to about 7 months, about 1 day to about 6 months, about 1 day to about 5 months, about 1 day to about 4 months, about 1 day to about 3 months, about 1 day to about 10 weeks, about 1 day to about 2 months, about 1 day to about 6 weeks, about 1 day to about 1 month, about 1 day to about 25 days, about 1 day to about 20 days, about 1 day to about 15 days, about 1 day to about 10 days, about 1 day to about 5 days, about 2 days to about 1 year, about 5 days to about 1 year, about 10 days to about 1 year, about 15 days to about 1 year, about 20 days to about 1 year, about 25 days to about 1 year, about 1 month to about 1 year, about 6 weeks to about 1 year, about 2 months to about 1 year, about 3 months to about 1 year, about 4 months to about 1 year, about 5 months to about 1 year, about 6 months to about 1 year, about 7 months to about 1 year, about 8 months to about 1 year, about 9 months to about 1 year, about 10 months to about 1 year, about 11 months to about 1 year, about 1 day to about 7 days, about 1 day to about 14 days, about 5 days to about 10 days, about 5 day to about 20 days, about 10 days to about 20 days, about 15 days to about 1 month, about 15 days to about 2 months, about 1 week to about 1 month, about 2 weeks to about 1 month, about 1 month to about 3 months, about 3 months to about 6 months, about 4 months to about 6 months, about 5 months to about 8 months, or about 7 months to about 9 months.

In some embodiments provided herein, a biomarker as described herein (e.g., HBV DNA, HBsAg, HBcAg, HBeAg, HDV DNA, or HDAg) is detected in a serum sample from the subject. In some embodiments, the level of a biomarker in a serum sample is compared to the level of the biomarker in a reference sample, e.g., a sample obtained from the subject prior to starting treatment with an NTCP inhibitor as described herein. In some embodiments, the reference sample is a serum sample from the subject (i.e., a serum sample obtained from the subject prior to starting treatment with an NTCP inhibitor).

Some embodiments of the methods described herein further include administering one or more additional anti-viral agents. Accordingly, also provided herein are methods of treating hepatitis B (HBV) and/or hepatitis D (HDV) in a subject in need thereof, the methods comprising administering to the subject one or more NTCP inhibitors (e.g., any of the NTCP inhibitors described herein) and one or more additional anti-viral agents. The one or more NTCP inhibitors (e.g., a compound of formula (I), or a pharmaceutically acceptable salt thereof, or a compound of formula (II), or a pharmaceutically acceptable salt thereof) and the one or more additional anti-viral agents can be administered simultaneously, sequentially or separately.

Non-limiting examples of additional anti-viral agents include nucleoside reverse transcriptase inhibitors, bulevirtide (also known as hepcludex or Myrcludex-B), and interferon. Non-limiting examples of nucleoside reverse transcriptase inhibitors include tenofovir (e.g., VIREAD®), tenofovir alafenamide (e.g., VEMLIDY®), tenofovir disoproxil, lamivudine (e.g., EPIVIR®, ZEFFIX®, or HEPTODIN®), entecavir (e.g., BARACLUDE®), abacavir (e.g., ZIAGEN®), stavudine (e.g., ZERIT®), didanosine (e.g., VIDEX®), telbivudine (e.g., TYZEKA® or SEBIVO®), zidovudine (e.g., RETROVIR®), zalcitabine (e.g., HIVID®), adefovir (e.g., HESPERA®), adefovir dipivoxil, and emtricitabin (e.g., EMTRIVA®). Non-limiting examples of interferons include pegylated interferon (e.g., PEGASYS®) and interferon alpha (e.g., Intron A).

Also provided herein are methods of treating hepatitis B and/or D, comprising administering to a subject in need thereof a compound of formula I, or a pharmaceutically acceptable salt thereof, and an additional anti-viral agent (e.g., any of the additional anti-viral agents described herein), wherein the amounts of the compound of formula I, or a pharmaceutically acceptable salt thereof, and the additional anti-viral agent are together effective in treating hepatitis.

Also provided herein are methods of treating hepatitis B and/or D, comprising administering to a subject in need thereof a compound of formula II, or a pharmaceutically acceptable salt thereof, and an additional anti-viral agent (e.g., any of the additional anti-viral agents described herein), wherein the amounts of the compound of formula I, or a pharmaceutically acceptable salt thereof, and the additional anti-viral agent are together effective in treating hepatitis.

In some embodiments, the additional anti-viral agent is tenofovir disoproxil.

Also provided herein are methods of treating hepatitis B and/or D, comprising administering to a subject in need thereof a compound of formula I, or a pharmaceutically acceptable salt thereof, and tenofovir disoproxil, wherein the amounts of the compound of formula I, or a pharmaceutically acceptable salt thereof, and tenofovir disoproxil are together effective in treating hepatitis.

Also provided herein are methods of treating hepatitis B and/or D, comprising administering to a subject in need thereof a compound of formula II, or a pharmaceutically acceptable salt thereof, and tenofovir disoproxil, wherein the amounts of the compound of formula II, or a pharmaceutically acceptable salt thereof, and tenofovir disoproxil are together effective in treating hepatitis.

Also provided herein is a NTCP inhibitor (such as a compound of formula (I), or a pharmaceutically acceptable salt thereof, or a compound of formula (II), or a pharmaceutically acceptable salt thereof), for use in treating hepatitis B (HBV) and/or hepatitis D (HDV).

Also provided herein is a NTCP inhibitor (such as a compound of formula (I), or a pharmaceutically acceptable salt thereof, or a compound of formula (II), or a pharmaceutically acceptable salt thereof), for use in preventing or decreasing entry of a hepatitis B viral particle and/or hepatitis D viral particle into a hepatocyte.

Also provided herein is a NTCP inhibitor (such as a compound of formula (I), or a pharmaceutically acceptable salt thereof, or a compound of formula (II), or a pharmaceutically acceptable salt thereof), for use in decreasing hepatitis B and/or hepatitis D viral replication in a hepatocyte.

Also provided herein is the use of a NTCP inhibitor (such as a compound of formula (I), or a pharmaceutically acceptable salt thereof, or a compound of formula (II), or a pharmaceutically acceptable salt thereof), in the manufacture of a medicament for the treatment of hepatitis B (HBV) and/or hepatitis D (HDV).

Also provided herein is the use of a NTCP inhibitor (such as a compound of formula (I), or a pharmaceutically acceptable salt thereof, or a compound of formula (II), or a pharmaceutically acceptable salt thereof), in the manufacture of a medicament for preventing or decreasing entry of a hepatitis B viral particle and/or hepatitis D viral particle into a hepatocyte.

Also provided herein is the use of a NTCP inhibitor (such as a compound of formula (I), or a pharmaceutically acceptable salt thereof, or a compound of formula (II), or a pharmaceutically acceptable salt thereof), in the manufacture of a medicament for decreasing hepatitis B and/or hepatitis D viral replication in a hepatocyte.

In some embodiments, the methods described herein comprise administering an NTCP inhibitor as described herein (e.g., a compound of formula (I), or a pharmaceutically acceptable salt thereof, or a compound of formula (II), or a pharmaceutically acceptable salt thereof), as a pharmaceutical composition that includes the NTCP inhibitor and one or more pharmaceutically acceptable excipients, and optionally one or more additional therapeutic agents as described herein. In some embodiments, the method comprises orally administering the NTCP inhibitor (e.g., a compound of formula (I), or a pharmaceutically acceptable salt thereof, or a compound of formula (II), or a pharmaceutically acceptable salt thereof), as a pharmaceutical composition.

In some embodiments, the pharmaceutical composition comprises a therapeutically effective amount of an NTCP inhibitor as described herein (e.g., a compound of formula (I), or a pharmaceutically acceptable salt thereof, or a compound of formula (II), or a pharmaceutically acceptable salt thereof), and one or more pharmaceutically acceptable excipients. The excipients can include, but are not limited to, fillers, binders, disintegrants, glidants and lubricants. In general, pharmaceutical compositions may be prepared in a conventional manner using conventional excipients.

Examples of suitable fillers include, but are not limited to, dicalcium phosphate dihydrate, calcium sulfate, lactose (such as lactose monohydrate), sucrose, mannitol, sorbitol, cellulose, microcrystalline cellulose, dry starch, hydrolyzed starches and pregelatinized starch. In certain embodiments, the filler is mannitol and/or microcrystalline cellulose.

Examples of suitable binders include, but are not limited to, starch, pregelatinized starch, gelatin, sugars (such as sucrose, glucose, dextrose, lactose and sorbitol), polyethylene glycol, waxes, natural and synthetic gums (such as acacia gum and tragacanth gum), sodium alginate, cellulose derivatives (such as hydroxypropylmethylcellulose (or hypromellose), hydroxypropylcellulose and ethylcellulose) and synthetic polymers (such as acrylic acid and methacrylic acid copolymers, methacrylic acid copolymers, methyl methacrylate copolymers, aminoalkyl methacrylate copolymers, polyacrylic acid/polymethacrylic acid copolymers and polyvinylpyrrolidone (povidone)). In certain embodiments, the binder is hydroxypropylmethylcellulose (hypromellose).

Examples of suitable disintegrants include, but are not limited to, dry starch, modified starch (such as (partially) pregelatinized starch, sodium starch glycolate and sodium carboxymethyl starch), alginic acid, cellulose derivatives (such as sodium carboxymethylcellulose, hydroxypropyl cellulose, and low substituted hydroxypropyl cellulose (L-HPC)) and cross-linked polymers (such as carmellose, croscarmellose sodium, carmellose calcium and cross-linked PVP (crospovidone)). In certain embodiments, the disintegrant is croscarmellose sodium.

Examples of suitable glidants and lubricants include, but are not limited to, talc, magnesium stearate, calcium stearate, stearic acid, glyceryl behenate, colloidal silica, aqueous silicon dioxide, synthetic magnesium silicate, fine granulated silicon oxide, starch, sodium lauryl sulfate, boric acid, magnesium oxide, waxes (such as carnauba wax), hydrogenated oil, polyethylene glycol, sodium benzoate, polyethylene glycol, and mineral oil. In certain embodiments, the glidant or lubricant is magnesium stearate or colloidal silica.

The pharmaceutical composition may be conventionally coated with one or more coating layers. Enteric coating layers or coating layers for delayed or targeted release of the compound of formula (I) or formula (II), or pharmaceutically acceptable salts thereof, are also contemplated. The coating layers may comprise one or more coating agents, and may optionally comprise plasticizers and/or pigments (or colorants).

Example of suitable coating agents include, but are not limited to, cellulose-based polymers (such as ethylcellulose, hydroxypropylmethylcellulose (or hypromellose), hydroxypropylcellulo se, cellulose acetate phthalate, cellulose acetate succinate, hydroxypropyl methylcellulose acetate succinate and hydroxypropyl methylcellulose phthalate), vinyl-based polymers (such as polyvinyl alcohol) and polymers based on acrylic acid and derivatives thereof (such as acrylic acid and methacrylic acid copolymers, methacrylic acid copolymers, methyl methacrylate copolymers, aminoalkyl methacrylate copolymers, polyacrylic acid/polymethacrylic acid copolymers). In certain embodiments, the coating agent is hydroxypropylmethylcellulose. In other embodiments, the coating agent is polyvinyl alcohol.

Examples of suitable plasticizers include, but are not limited to, triethyl citrate, glyceryl triacetate, tributyl citrate, diethyl phthalate, acetyl tributyl citrate, dibutyl phthalate, dibutyl sebacate and polyethylene glycol. In certain embodiments, the plasticizer is polyethylene glycol.

Examples of suitable pigments include, but are not limited to, titanium dioxide, iron oxides (such as yellow, brown, red or black iron oxides) and barium sulfate.

The pharmaceutical composition may be in a form that is suitable for oral administration, for parenteral injection (including intravenous, subcutaneous, intramuscular and intravascular injection), for topical administration, or for rectal administration. In some embodiments, the pharmaceutical composition is in a form that is suitable for oral administration. Pharmaceutical compositions formulated for oral administration can include, e.g., tablets and capsules.

The dosage required for the therapeutic or prophylactic treatment will depend on the route of administration, the severity of the disease, the age and weight of the patient and other factors normally considered by the attending physician, when determining the appropriate regimen and dosage level for a particular patient.

The amount of the NTCP inhibitor (e.g., a compound of formula (I), or a pharmaceutically acceptable salt thereof, or a compound of formula (II), or a pharmaceutically acceptable salt thereof) to be administered will vary for the subject being treated, and may vary from about 1 μg/kg of body weight to about 50 mg/kg of body weight per day. A unit dose form, such as a tablet or capsule, will usually contain about 1 to about 250 mg of active ingredient, such as about 1 to about 100 mg, or such as about 1 to about 50 mg, or such as about 1 to about 20 mg, e.g. about 2.5 mg, or about 5 mg, or about 10 mg, or about 15 mg. The daily dose can be administered as a single dose or divided into one, two, three or more unit doses. An orally administered daily dose of a bile acid modulator is preferably within about 0.1 to about 250 mg, more preferably within about 1 to about 100 mg, such as within about 1 to about 5 mg, such as within about 1 to about 10 mg, such as within about 1 to about 15 mg, or such as within about 1 to about 20 mg.

Some compounds of formula (I) or formula (II), or pharmaceutically acceptable salts thereof, may show a higher free fraction in plasma. In some embodiments, the free fraction is greater than about 0.2%, such as greater than about 0.4%, such as greater than about 0.6%, such as greater than about 0.8%, such as greater than about 1.0%, such as greater than about 1.25%, such as greater than about 1.5%, such as greater than about 1.75%, such as greater than about 2.0%, such as greater than about 2.5%, such as greater than about 3%, such as greater than about 4%, such as greater than about 5%, such as greater than about 7.5%, such as greater than about 10%, or such as greater than about 20%.

Some compounds of formula (I) or formula (II), or pharmaceutically acceptable salts thereof, may be excreted in urine. In some embodiments, the fraction of the compound that is excreted in urine is greater than about 0.2%, such as greater than about 0.4%, such as greater than about 0.6%, such as greater than about 0.8%, such as greater than about 1.0%, such as greater than about 2%, such as greater than about 3%, such as greater than about 5%, such as greater than about 7.5%, such as greater than about 10%, such as greater than about 15%, such as greater than about 20%, such as greater than about 30%, or such as greater than about 50%.

Following absorption from the intestine, some compounds of formula (I) or formula (II), or pharmaceutically acceptable salts thereof, may be circulated via the enterohepatic circulation. In some embodiments, the fraction of the compound that is circulated via the enterohepatic circulation is greater than about 0.1%, such as greater than about 0.2%, such as greater than about 0.3%, such as greater than about 0.5%, such as greater than about 1.0%, such as greater than about 1.5%, such as greater than about 2%, such as greater than about 3%, such as greater than about 5%, such as greater than about 7%, such as greater than about 10%, such as greater than about 15%, such as greater than about 20%, such as greater than about 30% or such as greater than about 50%.

Some compounds of formula (I) or formula (II), or pharmaceutically acceptable salts thereof, may cause renal excretion of bile salts. In some embodiments, the fraction of circulating bile acids that is excreted by the renal route is greater than about 1%, such as greater than about 2%, such as greater than about 5%, such as greater than about 7%, such as greater than about 10%, such as greater than about 15%, such as greater than about 20%, or such as greater than about 25%.

Some compounds of formula (I) or formula (II), or pharmaceutically acceptable salts thereof, may show improved or optimal permeability. The permeability may be measured in Caco2 cells, and values are given as Papp (apparent permeability) values in cm/s. In some embodiments, the permeability is greater than at least about 0.1×10⁻⁶ cm/s, such as greater than about 0.2×10⁻⁶ cm/s, such as greater than about 0.4×10⁻⁶ cm/s, such as greater than about 0.7×10⁻⁶ cm/s, such as greater than about 1.0×10⁻⁶ cm/s, such as greater than about 2×10⁻⁶ cm/s, such as greater than about 3×10⁻⁶ cm/s, such as greater than about 5×10⁻⁶ cm/s, such as greater than about 7×10⁻⁶ cm/s, such as greater than about 10×10⁻⁶ cm/s, such as greater than about 15×10⁻⁶ cm/s.

Some compounds of formula (I) or formula (II), or pharmaceutically acceptable salts thereof, may show an improved or optimal bioavailability. In some embodiments, the oral bioavailability is greater than about 5%, such as greater than about 7%, such as greater than about 10%, such as greater than about 15%, such as greater than about 20%, such as greater than about 30%, such as greater than about 40%, such as greater than about 50%, such as greater than about 60%, such as greater than about 70% or such as greater than about 80%. In other embodiments, the oral bioavailability is between about 10 and about 90%, such as between about 20 and about 80%, such as between about 30 and about 70% or such as between about 40 and about 60%.

Some compounds of formula (I) or formula (II), or pharmaceutically acceptable salts thereof, may be a substrate to relevant transporters in the kidney.

Also provided herein are methods for preventing or decreasing entry of a hepatitis B viral particle and/or hepatitis D viral particle into a cell, the method comprising contacting the cell with an NTCP inhibitor as described herein (e.g., a compound of formula (I), or a pharmaceutically acceptable salt thereof, or a compound of formula (II), or a pharmaceutically acceptable salt thereof). Also provided herein are methods for decreasing hepatitis B and/or hepatitis D viral replication in a cell, the method comprising contacting the cell with an NTCP inhibitor as described herein (e.g., a compound of formula (I), or a pharmaceutically acceptable salt thereof, or a compound of formula (II), or a pharmaceutically acceptable salt thereof). Also provided herein are methods for preventing binding of the preS1 domain of the L protein of HBV to NTCP in a cell, the method comprising contacting the cell with an NTCP inhibitor as described herein (e.g., a compound of formula (I), or a pharmaceutically acceptable salt thereof, or a compound of formula (II), or a pharmaceutically acceptable salt thereof). In some embodiments, the contacting is in vitro. In some embodiments, the contacting is in vivo. In some embodiments, the cell is a hepatocyte. In some embodiments, the contacting is in vivo, wherein the method comprises administering an effective amount of an NTCP inhibitor as described herein (e.g., a compound of formula (I), or a pharmaceutically acceptable salt thereof, or a compound of formula (II), or a pharmaceutically acceptable salt thereof), to a hepatocyte of a subject.

### Definitions

As used herein, the term “halo” refers to fluoro, chloro, bromo and iodo.

As used herein, the term “C₁₋₆ alkyl” refers to a straight or branched alkyl group having from 1 to 6 carbon atoms, and the term “C₁₋₄ alkyl” refers to a straight or branched alkyl group having from 1 to 4 carbon atoms. Examples of C₁₋₄ alkyl include methyl, ethyl, n-propyl, isopropyl, n-butyl, isobutyl, sec-butyl and tert-butyl.

As used herein, the term “C₁₋₄ haloalkyl” refers to a straight or branched C₁₋₄ alkyl group, as defined herein, wherein one or more hydrogen atoms have been replaced with halogen. Examples of C₁₋₄ haloalkyl include chloromethyl, fluoroethyl and trifluoromethyl.

As used herein, the terms “C₁₋₄ alkoxy” and “C₁₋₄ alkylthio” refer to a straight or branched C₁₋₄ alkyl group attached to the remainder of the molecule through an oxygen or sulphur atom, respectively.

As used herein, the term “C₃₋₆ cycloalkyl” refers to a monocyclic saturated hydrocarbon ring having from 3 to 6 carbon atoms. Examples of C₃₋₆ cycloalkyl include cyclopropyl, cyclobutyl, cyclopentyl and cyclohexyl.

The term “aryl” denotes an aromatic monocyclic ring composed of 6 carbon atoms or an aromatic bicyclic ring system composed of 10 carbon atoms. Examples of aryl include phenyl, naphthyl and azulenyl.

The term “amino” refers to an —NH₂ group. As used herein, the terms “N-(C₁₋₄ alkyl)amino” and “N,N-di(C₁₋₄ alkyl)amino” refer to an amino group wherein one or both hydrogen atom(s), respectively, are replaced with a straight or branched C₁₋₄ alkyl group. Examples of N-(C₁₋₄ alkyl)amino include methylamino, ethylamino and tert-butylamino, and examples of N,N-di-(C₁₋₄ alkyl)amino include dimethylamino and diethylamino.

As used herein, the term “N-(aryl-C₁₋₄ alkyl)amino” refers to an amino group wherein a hydrogen atom is replaced with an aryl-C₁₋₄ alkyl group. Examples of N-(aryl-C₁₋₄ alkyl)amino include benzylamino and phenylethylamino. The term “C₁₋₆ alkylcarbonylamino” refers to an amino group wherein a hydrogen atom is replaced with a C₁₋₆ alkylcarbonyl group. Examples of C₁₋₆ alkanoylamino include acetylamino and tert-butylcarbonylamino. The term “C₁₋₄ alkyloxycarbonylamino” refers to an amino group wherein a hydrogen atom is replaced with a C₁₋₄ alkyloxycarbonyl group. An example of C₁₋₄ alkyloxycarbonylamino is tert-butoxycarbonylamino. The terms “C₁₋₄ alkylsulfonamido” and “C₃₋₆ cycloalkylsulfonamido” refer to an amino group wherein a hydrogen atom is replaced with a C₁₋₄ alkylsulfonyl or a C₃₋₆ cycloalkylsulfonyl group, respectively.

Some compounds of formula (I) or formula (II), or pharmaceutically acceptable salts thereof, may have chiral centers and/or geometric isomeric centers (E- and Z-isomers). It is to be understood that the invention encompasses all such optical isomers, diastereoisomers and geometric isomers that possess ASBT and/or LBAT inhibitory activity. The invention also encompasses any and all tautomeric forms of compounds of formula (I) or formula (II), or pharmaceutically acceptable salts thereof, that possess ASBT and/or LBAT inhibitory activity. Certain compounds of formula (I) or formula (II), or pharmaceutically acceptable salts thereof, may exist in unsolvated as well as solvated forms, such as, for example, hydrated forms. It is to be understood that the invention encompasses all such solvated forms that possess ASBT and/or LBAT inhibitory activity.

As used herein, the term “pharmaceutically acceptable” refers to those compounds, materials, compositions and/or dosage forms that are suitable for human pharmaceutical use and that are generally safe, non-toxic and neither biologically nor otherwise undesirable.

A suitable pharmaceutically acceptable salt of a compound disclosed herein is, for example, a base-addition salt of the compound which is sufficiently acidic, such as an alkali metal salt (e.g., a sodium or potassium salt), an alkaline earth metal salt (e.g., a calcium or magnesium salt), an ammonium salt, or a salt with an organic base which affords a physiologically acceptable cation, for example a salt with methylamine, dimethylamine, trimethylamine, piperidine, morpholine or tris-(2-hydroxyethyl)amine.

As used herein, the terms “treatment”, “treat”, and “treating” refer to reversing, alleviating, delaying the onset of, or inhibiting the progress of a disease or disorder, or one or more symptoms thereof, as described herein. In some embodiments, an NTCP inhibitor is administered after one or more symptoms have developed. In other embodiments, an NTCP inhibitor may be administered in the absence of symptoms. For example, an NTCP inhibitor may be administered to a susceptible individual prior to the onset of symptoms (e.g., in light of a history of symptoms and/or in light of genetic or other susceptibility factors). Administration of an NTCP inhibitor may also be continued after symptoms have resolved, for example to prevent or delay their recurrence.

As used herein, the terms “subject,” “individual,” or “patient,” are used interchangeably, refers to any animal, including mammals such as mice, rats, other rodents, rabbits, dogs, cats, swine, cattle, sheep, horses, primates, and humans. In some embodiments, the subject is a human. In some embodiments, the subject has experienced and/or exhibited at least one symptom of hepatitis B and/or hepatitis D.

As used herein, the term “contacting” refers to the bringing together of indicated moieties in an in vitro system or an in vivo system. For example, “contacting” NTCP with a compound provided herein includes the administration of a compound provided herein to subject, such as a human, having NTCP, as well as, for example, introducing a compound provided herein into a sample containing a cellular or purified preparation containing NTCP.

As used herein, the term “about” refers to a value or parameter herein that includes (and describes) embodiments that are directed to that value or parameter per se. For example, description referring to “about 20” includes description of “20.” Numeric ranges are inclusive of the numbers defining the range. Generally speaking, the term “about” refers to the indicated value of the variable and to all values of the variable that are within the experimental error of the indicated value (e.g., within the 95% confidence interval for the mean) or within 10 percent of the indicated value, whichever is greater.

### EXAMPLES

### Example 1. Inhibition of NTCP and IBAT In Vitro

The effect of (Z)-3-((3-butyl-2- methyl-7-(methylthio)-1,1-dioxido-5-phenyl-2,3,4,5-tetrahydro-1,2,5-benzothiadiazepin-8-yl)oxy)-2-fluoroacrylic acid (Compound 1) on NTCP- and IBAT-mediated bile acid transport using an over-expressed cell line system was evaluated.

### Methods

### Assay Procedure for NTCP

20,000 cells (Human & Mouse NTCP-overexpressing cells) were seeded in 96-well plate in 100 μL MEM-alpha medium supplemented with 10% FBS containing geneticin (1 mg/mL) and incubated at 37° C. in 5% CO₂ for 24 hrs. After incubation, media was decanted from the wells and cells were washed two times with 250 μL of basal MEM-alpha medium (FBS-free). After each wash, plates were tapped against a paper towel to ensure maximum removal of residual media. For Human NTCP (hNTCP), incubation mix was prepared by adding test inhibitor dilutions (3-fold serial dilution in DMSO, 10 concentrations) in MEM-alpha (without FBS) containing 0.3 μM ³H-taurocholic acid and 7.5 μM cold taurocholic acid (maintaining 0.2% final DMSO concentration). For Mouse NTCP, incubation mix was prepared by adding test inhibitor dilutions (3-fold serial dilution in DMSO, 10 concentrations) in MEM-alpha (without FBS) containing 0.3 μM ³H-taurocholic acid and 25 μM cold taurocholic acid (maintaining 0.2% final DMSO concentration). 50 μL of incubation mix containing test inhibitors was then added to the wells (in duplicate) and the plates were incubated for 20 min in a CO₂ incubator at 37° C. The wells were washed two times with 250 μL of chilled un-labelled 1 mM taurocholic acid dissolved in HEPES-buffered (10 mM) HBSS (pH 7.4).

The plates were tapped against a paper towel after every wash to ensure maximum removal of blocking buffer. 100 μL of MicroScint-20 was added to the wells and kept overnight at room temperature before reading the plates in MicroBeta2 liquid Scintillation Counter from PerkinElmer under 3H Test for I/L-BAT protocol (set at 120 seconds reading time per well, with normal plate orientation).

### Assay Procedure for IBAT

10,000 cells (Human & Mouse IBAT-overexpressing cells) were seeded in 96-wells plate in 100 μL MEM-alpha medium supplemented with 10% FBS containing puromycin (10 μg/mL) and incubated at 37° C. in 5% CO₂ for 48 hrs. After incubation media was decanted from the wells, cells were washed two times with 250 μL of basal MEM-alpha medium (FBS-free). After each wash, plates were tapped against paper towel to ensure maximum removal of residual media. Test inhibitor dilutions (highest test concentration being 10 μM, 3-fold serial dilution, 10 concentrations) prepared in DMSO were added to the incubation mix (maintaining 0.2% final DMSO concentration) containing 0.25 μM ³H-taurocholic acid and 5 μM of cold taurocholic acid. 50 μL of incubation mix containing test inhibitors was then added to the wells (in duplicate) and the plates were incubated for 20 min in a CO₂ incubator at 37° C.

After incubation, the reaction was stopped by keeping the plates on ice water mix for 2-3 minutes and then the incubation mix was aspirated completely from the wells. The wells were washed two times with 250 μL of chilled un-labelled 1 mM taurocholic acid dissolved in HEPES-buffered (10 mM) HBSS (pH 7.4). The plates were tapped against a paper towel after every wash to ensure maximum removal of blocking buffer. 100 μL of MicroScint-20 was added to the wells and kept overnight at room temperature before reading the plates in MicroBeta2 liquid Scintillation Counter from PerkinElmer under ³H Test for I/L-BAT protocol (set at 120 seconds reading time per well, with normal plate orientation).

### Statistical Analysis

Percentage inhibitions were calculated with respect to the assay controls. Further data analysis was performed using validated statistical software (GraphPad Prism) to calculate the IC₅₀ values for the test compound.

### Results

The IC₅₀ value for Compound 1 in human NTCP was 0.53±0.081 nM. The positive control myrcludex B (MyrB) behaved as expected.

The IC₅₀ value for Compound 1 in mouse NTCP was 1.82±0.25 nM. The positive control MyrB behaved as expected.

The IC₅₀ value for Compound 1 in human IBAT was 535±83.3 nM. The positive control AS0075 behaved as expected.

The IC₅₀ value for Compound 1 in mouse IBAT was 40.8±4.6 nM. The positive control AS0075 behaved as expected.

### Example 2. Inhibition of NTCP and HBV In Vitro

The in vitro potency of Compound lto inhibit bile acid transport was evaluated.

### Methods

### Assay Procedure in CHO Cells

Stably hNTCP-transfected CHO cells were seeded onto 24 well plates. After overnight incubation, CHO cells were treated with 10 mM sodium butyrate to induce NTCP transporter expression. Twenty-four hours later, media was aspirated, cells were washed with warm Hanks balanced salt solution (HBSS) containing sodium (+Na). Cells were incubated with increasing doses of Compound 1 (for example: 0, 10 nM, 100 nM, 1 μM, 10 μM), in the presence of tracer doses of 5 μm, 10 μM, or 25 μM [³H]taurocholate at 37° C. for <10 minutes. The cells were processed for radioactivity. The apparent Kᵢ and kinetic types of inhibition (competitive, uncompetitive, non-competitive, mixed) were determined by complementary Dixon/Cornish-Bowden plot analysis and Lineweaver-Burk plot analysis.

### In vitro Evaluation of the Time Course of Dissociation

This study was performed using a [³H]taurocholate uptake assay protocol. Stably transfected CHO cells expressing human NTCP (hNTCP) were seeded onto 24 well plates at a density of 1.5-2.0×105 cells/well in CHO cell media (DMEM/F12 media supplemented with 10% FBS, Penicillin/Streptomycin) plus Geneticin (G418) to maintain selection in culture. After overnight incubation, the media was replaced, and CHO cells were treated with 10 mM sodium butyrate to induce transporter expression (final concentration=10 μM). Twenty-four h later, the media was removed, and cells were washed with warm (37° C.) Hanks balanced salt solution (HBSS) containing sodium (Na+). Cells were then incubated with 1 μM of the test inhibitors or vehicle (DMSO) for 15 min in CHO cell media at 37° C. Following the 15 min inhibitor incubation period, media was removed, and the cells were washed again with warm Hanks balanced salt solution (HBSS) containing sodium (Na+). New CHO cell media (without compound) was added on the cells to begin the inhibitor “washout” period and the plates were returned to the tissue culture incubator.

For the [³H]taurocholate uptake assay, the media was aspirated at the end of each washout period, and cells were washed three times with warm Hanks balanced salt solution (HBSS) containing sodium (Na+). Cells were then incubated in HBSS containing 5 μM [³H]taurocholate at 37° C. on a slide warmer for 10 minutes. For the 0 minute timepoint, 1 μM of the test inhibitor was present during the 10 minute [³H]taurocholate uptake period. The cells were then moved onto ice, and the [³H]taurocholate plus inhibitor mix was removed by aspiration. The cells were washed with ice-cold HBSS+Choline (in place of Na+) and lysed with 0.1 N NaOH. Duplicate 100 μL aliquots of the lysate were transferred to vials with scintillation liquid and counted after neutralization and overnight quenching. Cell-associated protein was measured in duplicate using the BCA protein assay, and cell-associated radioactivity was normalized to protein content.

### Results

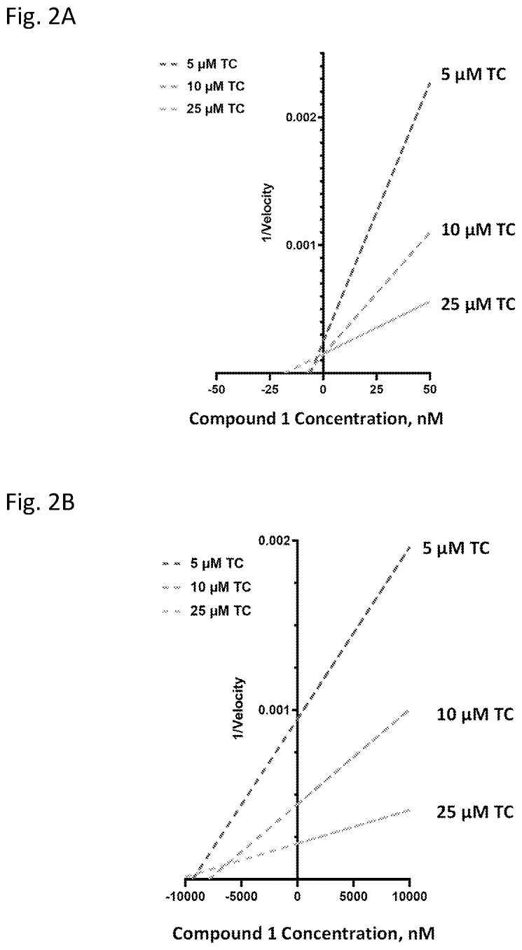

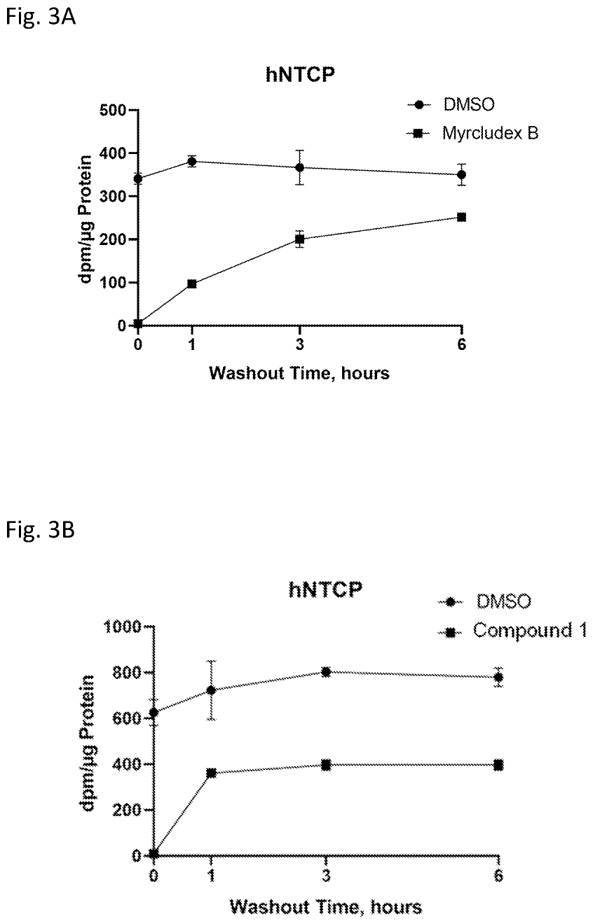

Compound 1 acted as a competitive inhibitor of transporter activity, with inhibitory constant values (Kᵢ) of 15 nmol/L and 2410 nmol/L for human NTCP (FIG. 2A) and ASBT (FIG. 2B), respectively. In vitro experiments demonstrated that Compound 1 displayed long-lasting inhibition of human NTCP BA transport activity (>50% inhibition at 6 hours postwashout) (FIGS. 3A (Myrcludex B) and 3B (Compound 1)).

### Example 3. HBV/HDV-Dervied preS1-Peptide Binding to NTCP

Myristoylated preS1 domain, comprising amino acids 2-48 of the large HBV envelope, is essential for virus binding to NTCP and can be used as surrogate parameter for HBV/HDV virus binding to NTCP. In this in vitro study, the potency and selectivity of Compound 1 to inhibit taurocholic acid transport via human NTCP and Macaca fascicularis Ntcp, HBV/HDV preS1-peptide binding to human NTCP and in vitro HDV and HBV infection of NTCP-HepG2 cells were investigated.

### Methods

### NTCP-HEK293 and NTCP-HepG2 Cell Lines

Human embryonic kidney (HEK293) cells were stably transfected with human NTCP, C-terminally tagged with the FLAG epitope (here referred to as NTCP-HEK293 cells). Cells were maintained at 37° C., 5% CO₂ and 95% humidity in DMEM/F-12 medium (Thermo Fisher Scientific, Waltham, Mass., USA) supplemented with 10% fetal calf serum (Sigma-Aldrich, St. Louis, Mo., USA), 4 mM L-glutamine, and penicillin/streptomycin. HepG2 cells stably transfected with NTCP-FLAG (here referred to as NTCP-HepG2) were cultured under the same conditions in DMEM with all supplements listed above, except for L-glutamine. For induction of the transgene, the medium was supplemented with 1 μg/mL tetracycline in the case of the NTCP-HEK293 cells or with 2 μg/mL doxycycline in the case of the NTCP-HepG2 cells. See also König A, et al. (2014) Kinetics of the bile acid transporter and hepatitis B virus receptor Na+/taurocholate cotransporting polypeptide (NTCP) in hepatocytes. Journal of Hepatology 61:867-875; Kirstgen M, et al. (2021) Hepatitis D Virus Entry Inhibitors Based on Repurposing Intestinal Bile Acid Reabsorption Inhibitors. Viruses 13(4):666; Kirstgen M, et al. (2021) Identification of novel HBV/HDV entry inhibitors by pharmacophore- and QSAR-guided virtual screening. Viruses 13:1489; and Grosser G, et al. (2021) Substrate Specificities and Inhibition Pattern of the Solute Carrier Family 10 Members NTCP, ASBT and SOAT. Front Mol Biosci 8:689757.

### M. fascicularis NTCP Transfected HEK293 Cells

GripTite 293 MSR cells (here referred to as HEK293 cells, Invitrogen), a modified HEK293 cell line expressing human macrophage scavenger receptor for stronger adhesion, were maintained at 37° C., 5% CO₂ and 95% humidity in DMEM/F-12 medium (Thermo Fisher Scientific) supplemented with 10% fetal calf serum (Sigma), 4 mM L-glutamine (PAA) and penicillin/streptomycin. HEK293 cells were transiently transfected with the M. fascicularis Ntcp cDNA construct for transport and peptide binding assays. Transfection was performed with Lipofectamine 2000 (Thermo Fisher Scientific). See also Müller SF, et al. (2018) Characterisation of the hepatitis B virus cross-species transmission pattern via Na+/taurocholate co-transporting polypeptides from 11 New World and Old World primate species. PLoS One 13(6):e0199200.

### Inhibitory Concentrations (IC₅₀) for [³H]TC Transport and [³H]preS1 Binding

Bile acid transport measurements were performed in the NTCP-HEK293 cells with tritium-labelled taurocholic acid (here referred to as [³H]TC) (20 Ci/mmol, 0.09 mCi/mL, Perkin Elmer, Waltham, USA). In parallel, peptide binding experiments were performed with a tritium-labelled myr-preS12-48 lipopeptide -HBV subgenotype D3- (here referred to as [³H]preS1) that was purchased from Pharmaron (120 Ci/mmol, 1 mCi/mL, Cardiff, UK). Briefly, cells were seeded onto polylysine-coated 96-well plates, induced with 1 μg tetracycline per mL, and grown to confluence over 72 h at 37° C. Then, cells were washed once with tempered phosphate-buffered saline (PBS, 137 mM NaCl, 2.7 mM KCl, 1.5 mM KH₂PO₄, 7.3 mM Na₂HPO₄, pH 7.4) at 37° C. and preincubated with 80 μL DMEM for 5 min at 37° C. The medium was replaced by 80 μL DMEM containing the respective inhibitor concentrations or solvent alone (100% uptake/binding control), and cells were further incubated for 5 min at 37° C. After this pre-incubation, bile acid transport experiments were started by adding 20 μL DMEM containing 5 μM [³H]TC (final concentration: 1 μM). Binding of [³H]preS1 was initiated by adding 20 μL DMEM containing 25 nM [³H]preS1 (final concentration: 5 nM). Experiments were stopped after 10 min by washing twice with ice-cold PBS. For 0% uptake/binding control, the NTCP-HEK293 cells were not induced with tetracycline (-tet). Cell-associated radioactivity of [³H]TC or [³H]preS1 was quantified by liquid scintillation counting in a Packard Microplate Scintillation Counter TopCount NXT (Packard Instrument Company, Meriden, USA). Transport rates and [³H]preS1 binding were determined in counts per minute (cpm). The mean of the 0% control was subtracted and the net [³H]TC transport rates and net [³H]preS1 binding rates, respectively, were expressed as % of control. IC₅₀ values were calculated from quadruplicate determinations by GraphPad Prism 6 (GraphPad, San Diego, Calif., USA). See also Kirstgen et al. 2020, 2021, Grosser et al. 2021.

### MTT Cytotoxicity Assay

The In Vitro Toxicology Assay Kit (Sigma-Aldrich) was used to perform a 3-[4,5-dimethylthiazole-2-yl]-2,5-diphenyltetrazolium bromide (MTT) assay to measure the cytotoxicity of the test item according to the manufacturer's protocol. Briefly, NTCP-HepG2 cells were incubated with 100 μL of the indicated concentrations of the test item solved in Hepatocyte Growth Medium (HGM) over 6 h at 37° C. After 6 h, medium was replaced by inhibitor-free HGM and cells were cultured for additional 24 h. Then medium was removed and 100 μL DMEM containing 0.5 mg/mL MTT was added and cells were incubated for 1 h at 37° C. Finally, the medium was replaced by 100 μL isopropyl alcohol (Sigma-Aldrich) and samples were measured by Glo-Max-Multi Detection System (Promega, Madison, Wis., USA). See also Lowjaga K A A, et al. (2020). Long-term trans-inhibition of the hepatitis B and D virus receptor NTCP by taurolithocholic acid. Am J Physiol Gastrointest Liver Physiol 320(1):G66-G80; and Kirstgen et al. 2021.

### In Vitro HDV Infection

HDV (HDV genotype 1, enveloped with HBV surface proteins of genotype D, see HBV below) production was done in vitro as described in the literature (Rasche A, et al. (2019) Highly diversified shrew hepatitis B viruses corroborate ancient origins and divergent infection patterns of mammalian hepadnaviruses. Proc Natl Acad Sci U S A 116(34):17007-17012; De Carvalho Dominguez Souza B F, et al. (2018) A novel hepatitis B virus species discovered in capuchin monkeys sheds new light on the evolution of primate hepadnaviruses. Journal of Hepatology 68:1114-1122). RT-qPCR was performed to determine HDV RNA genome equivalents. NTCP-HepG2 cells were pre-incubated for 5 min with Compound 1 in HGM in concentrations of 100 nM, 200 nM, and 400 nM. Infection experiments were performed in NTCP-HepG2 cells. Cells were cultured in 48-well plates in HGM consisting of William's E Medium (Thermo Fisher Scientific) containing 2% bovine serum albumin (BSA, Roth), 2 mM L-Glutamine, 100 μg/mL gentamicin, 10 nM dexamethasone, 1 mM sodium pyruvate, 1× Insulin-Transferrin-Selen, 2% DMSO, 4% polyethylene glycol, and 2 μg/mL doxycycline. HDV stock was diluted in HGM (HDV: 2×109 GE/well) and was added to the cells for 6 h. Subsequently, cells were washed with DMEM and cultured in HGM supplemented with 2% DMSO, 2% BSA and 2 μg/mL doxycycline. Every three days medium was changed until cells were fixed at day 9 post infection with 3% paraformaldehyde in PBS, for 30 min at room temperature (RT). Cells were permeabilized with 0.2% Triton X 100 in PBS for 30 min at RT, and blocked by incubation with 5% bovine serum albumin in PBS, for 30 min at RT. Then, cells were immunostained with purified human anti-HDV-positive serum at 37° C. for 1 h (1:400 dilution). Goat anti-human IgG secondary antibody coupled to Alexa Fluor fluorophore (1:400 dilution, Thermo Fisher Scientific) was added for 1 h at 37° C. for detection of Hepatitis Delta antigen (HDAg). Nuclei were stained with Hoechst 33342 (1 μg/mL, Thermo Fisher Scientific). The number of infected cells per well was determined by fluorescence microscopy.

### In Vitro HBV Infection

In a first study, NTCP-HepG2 cells were inoculated for 16 h with similar HBV genome equivalents/cell. HBV (genotype D) was produced in vitro as described in the literature (König et al. 2014). Infection was done in HGM supplemented with 2% DMSO and 4% polyethylenglycol (5×109 GE/well HBV). Thereafter, cells were washed twice with HGM and cultured until day 10 post infection in HGM supplemented with 2% DMSO and 2% FCS. Fixation was performed at 10 days post infection with 3.7% formaldehyde and 1% methanol at 4° C. for 30 min. Cells were permeabilized with 0.2% Triton X100 in PBS for 20 min at room temperature. Unspecific binding epitopes were blocked by incubation with 10% fetal calf serum in PBS for 45 min at 37° C. For detection of HBV core (HBc) protein expression, cells were incubated for 2 h at 37° C. with a polyclonal rabbit anti-HBcAg antiserum (1:500 dilution, Dako, Hamburg, Germany) in PBS and thereafter with anti-rabbit IgG AlexaFluor594 (1:200 dilution in PBS, Immuno Jackson) for 1 hat 37° C. Nuclei were stained with DAPI (10 μg/mL) in PBS. The number of infected cells per well was determined by fluorescence microscopy. In addition, HBeAg was quantified in the supernatants from day 7 to day 11 post infection.

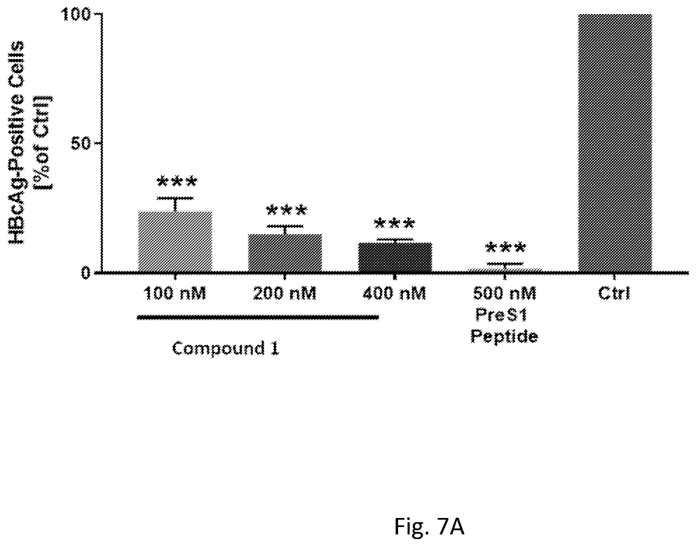

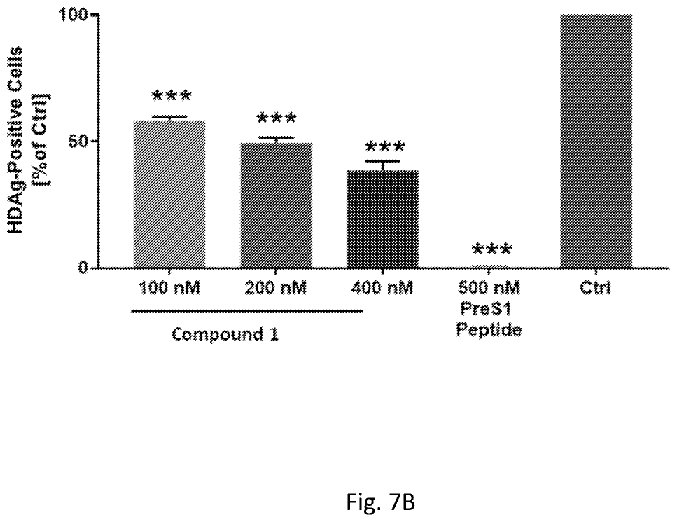

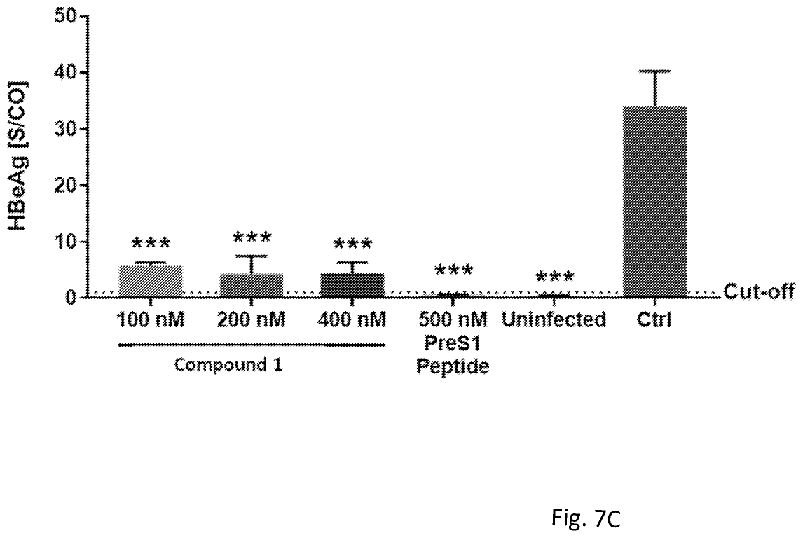

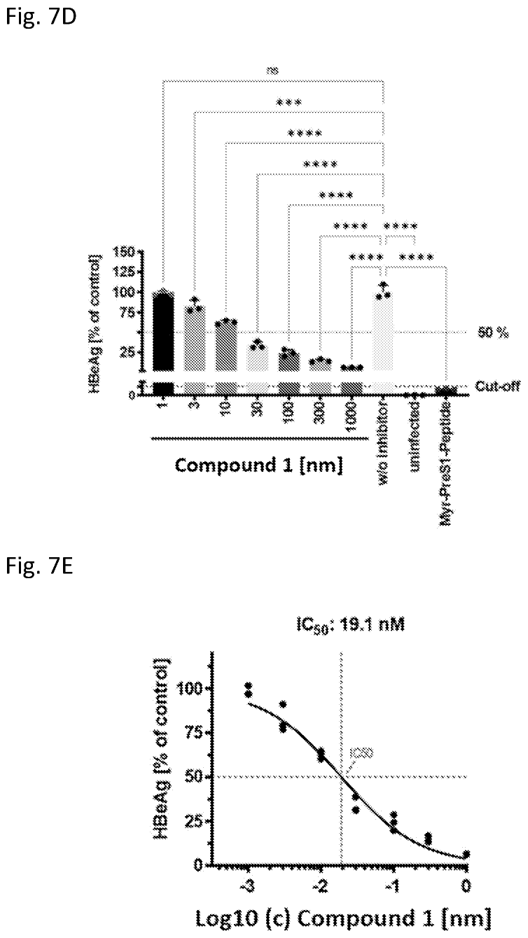

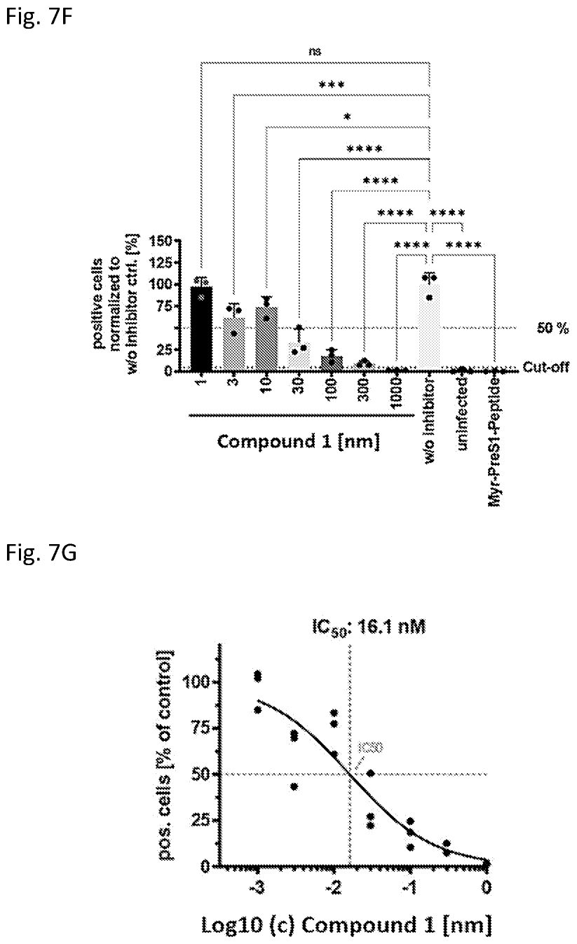

In a second study, recombinant HBV (genotype D) was produced in vitro as described in the literature (König et al. 2014). For infection experiments, NTCP-HepG2 cells (2×10⁵ cells per well) were pre-incubated for 5 min with Compound 1 or Myrcludex B in HGM at concentrations of 1, 3, 10, 30, 100, 300, and 1000 nM, respectively. See FIGS. 7D-7F. The myr-preS12-48 peptide (500 nM, genotype D) was used as control inhibitor. All infection experiments were performed in NTCP-HepG2 cells cultured in HGM in the presence of the corresponding compounds with different concentrations, 4% PEG-8000 (Sigma-Aldrich, Darmstadt, Germany), 2% DMSO (Carl Roth, Karlsruhe, Germany) and 100 ng/ml EGF (PeproTech, Hamburg, Germany) as final concentration each. Recombinant HBV was diluted in HGM to a final concentration of 2.5×10¹⁰ HBV-genome copies per well (7×10⁸ IU per well) and was added to the cells for 6 h. Subsequently, cells were washed with HGM and cultured in HGM supplemented with 2% DMSO. Medium was changed every 2-3 days until cells were fixed at day 9 p.i. with 3.8% paraformaldehyde (Sigma-Aldrich) in PBS, for 30 min at RT. The cells were immunostained at 37° C. for 1 h (1:400 dilution). For detection of HBV core (HBc) protein expression, a polyclonal anti-HBc antiserum from an immunized guinea pig (1:500 dilution, Eurogentec, Seraing, Belgium) was used as primary antibody and an Alexa Fluor 488-conjugated goat anti-guinea-pig-IgG-antibody (1:400 dilution, Thermo Fisher Scientific) as secondary antibody. Nuclei were stained with DAPI (0.5 μg/ml, Thermo Fisher Scientific). Analysis of immunofluorescence was performed with the ImageXpress Pico automated cell-imaging system (Molecular Devices, San José, USA). HBV-infected cells secrete the soluble HBeAg, which is a widely-accepted quantitative marker for productive HBV infection in cell culture experiments. In this study, HBeAg secreted from infected cells into the cell culture supernatant was determined of supernatant collected from day 5 to 9 p.i. using the Architect HBeAg assay, an automated in vitro diagnostic system (Abbott Laboratories, Wiesbaden, Germany).

### Statistics

Determination of IC₅₀ values was done by nonlinear regression analysis using the equation log(inhibitor) vs. response settings of the GraphPad Prism 6.0 software (GraphPad). Data of [³H]TC transport and [³H]preS1 binding are expressed as means±SD from quadruplicate determinations. Infection studies were performed in triplicate and data represent means±SD. Statistical analysis of the HBV/HDV infection experiments were performed by two-way ANOVA, followed by Dunnett's multiple comparison test by GraphPad Prism 9.0, considering p<0.01 as statistically significant.

### Results

### Inhibition of Human NTCP by Compound 1

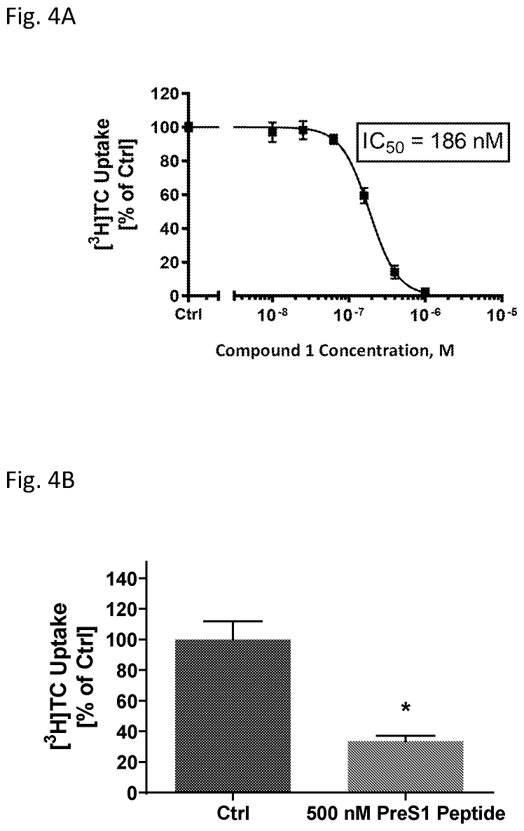

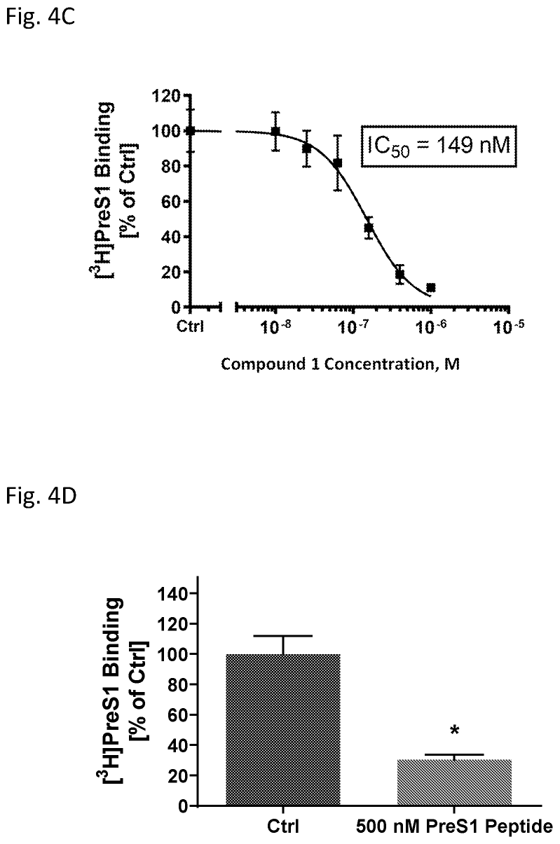

Compound 1 inhibited [3H]taurocholic acid uptake (half-maximal inhibitory concentration [IC₅₀], 186 nmol/L; FIGS. 4A) and [³H]preS1 peptide binding (IC₅₀, 149 nmol/L; FIGS. 4C) in a concentration-dependent manner. As control inhibitor 500 nM preS1 peptide was used in both assays, and significantly inhibited both assays as expected and as reported in the literature (see FIGS. 4B and 4D; König et al. 2014, Müller et al. 2018, Lowjaga et al. 2021).

### Inhibition of M. fascicularis Ntcp by Compound 1

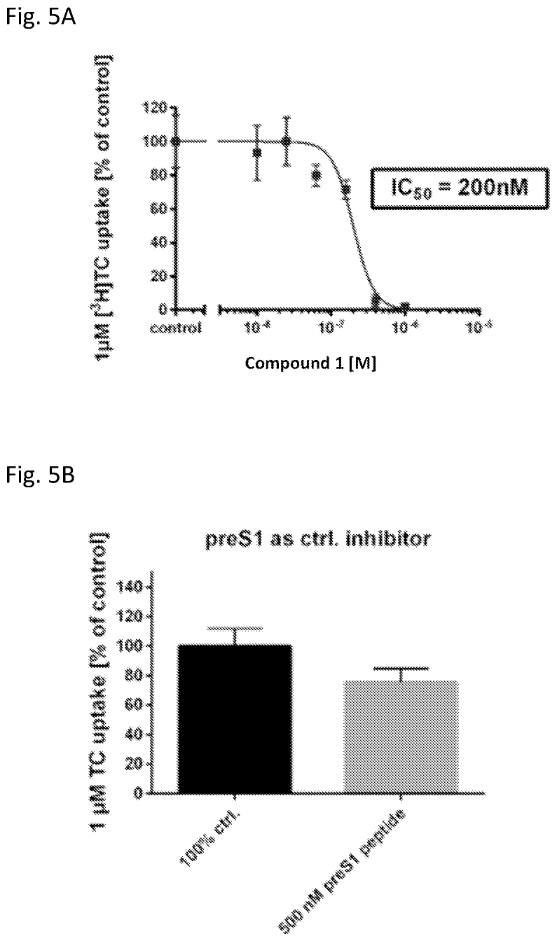

In addition, Compound 1 was used as inhibitor at the NTCP of Macaca fascicularis. This carrier was transiently transfected into HEK293 cells and the effect on [³H]TC transport inhibition was analyzed at increasing concentrations of 10, 25, 63, 158, 398, and 1000 nM. As shown in FIGS. 5A and 5B, Compound 1 potently inhibited the bile acid transport via M. fascicularis Ntcp in a concentration-dependent manner, with IC₅₀ of 200 nM.

### Cytotoxicity Assessment of Compound 1 in NTCP-HepG2 Cells

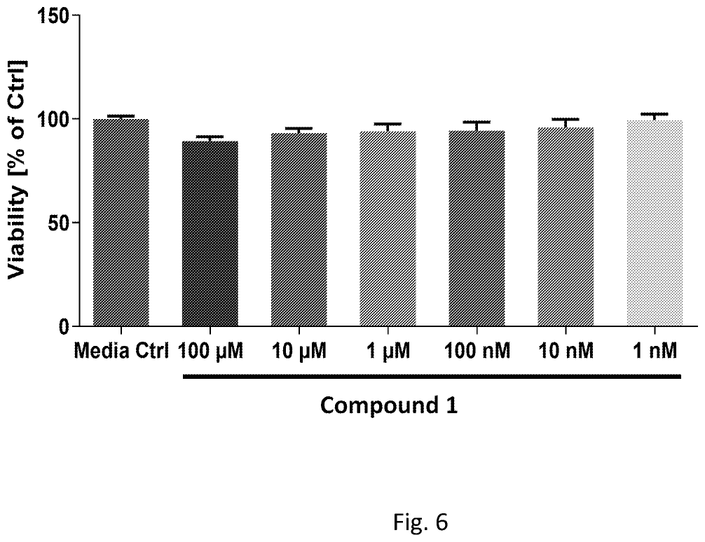

Cytotoxicity assessment of Compound 1 was done in HepG2 cells at a concentration range of 1 nM-100 μM. As shown in FIG. 6, Compound 1 showed no cytotoxic effects in HepG2 cells.

### Inhibition of In Vitro HBV/HDV Infection by Compound 1

In vitro inhibition of HBV/HDV infection by Compound 1 was analyzed in human NTCP-HepG2 hepatoma cells, which represent a well-established cell culture model for in vitro HBV/HDV infection (see König et al. 2014).

In the first study, different concentrations (100 nM, 200 nM, and 400 nM) of Compound 1 were used prior and during incubation of the virus inoculum with the cells. Compound 1 reduced the number of HBcAg-positive cells by 75%-90% (FIG. 7A), HDAg-positive cells by 40%-60% (FIG. 7B), and HBeAg levels in supernatant by 83%-87% (FIG. 7C). A similar approach with a preS1-peptide (aa 2-48, genotype D, 500 nM) resulted in almost complete inhibition of HDV/HBV infection. In the second study, NTCP-HepG2 cells were pre-incubated for 5 min with Compound 1 at concentrations of 1, 3, 10, 30, 100, 300, and 1000 nM. HBeAg secretion was above the cut off value even at the highest inhibitor concentration of 1000 nM in the case of compound A2342 (FIGS. 7D-7G). Nevertheless, an IC₅₀ value could be calculated. Compound 1 inhibited in vitro HBV infection with IC₅₀ values of 19.1 nM (HBeAg) (FIGS. 7D and 7E) and 16.1 nM (HBc) (FIGS. 7F and 7G).

### Discussion

The dual [³H]TC transport and [³H]preS1 peptide binding assay described in this Example has been used to characterize inhibitors of human NTCP regarding their inhibitory potency and selectivity towards virus binding inhibition (see Kirstgen et al. 2020, 2021). As the identical assay with the identical parameters was used in this Example y, the measured IC₅₀ values of Compound 1 can be directly compared to the published data. Compound 1 was equipotent to the viral preS1-peptide as inhibitor and was much more potent than the well-established NTCP inhibitor troglitazone.

At the selected concentrations, Compound 1 showed a strong inhibitory effect on HBV and HDV infection. Compound 1 potently blocked entry of HBV and HDV virus particles in NTCP-HepG2 cells in the nanomolar range.

### Example 4. Effect of Compound 1 on HBV Infection in Human Hepatocytes In Vitro

The antiviral effects of Compound 1 using HBV-infected human hepatocytes in vitro were evaluated.

### Methods

### Hepatocyte Incubations

A single vial of Primary Human Hepatocytes (PHH) was seeded in 48-well collagencoated plates according to the protocol provided by the hepatocyte cell provider (BiolVT). Cells were seeded at 0.14×106 cells in a total volume of 200 μL per well. The cells were incubated overnight at 37° C. in an environment of 5% CO₂ using InVitroGro HI media with Torpedo antibiotic mix. Unless otherwise stated, the diluted compounds were added to the cells 18 hours prior to infection (standard conditions).

Multiplicity of Infection (MOI) is defined as the number of HBV genome equivalents, as determined by the qPCR quantitation, added per cell in the respective growth format. The overnight media was removed from the cells and replaced with 190 μL of new, serum-free HI media/4% PEG8000 and 10 μL of the diluted virus stock (HepG2 AD38 genotype D) for a MOI of 500. Medium alone was added to the cells containing compound for the parallel cytotoxicity evaluation. After 18 hours of virus infection, the cells were gently washed five times with DPBS, followed by a new addition of 200 μL HI media containing compounds on day 1, day 4 and day 7 post-infection. In one set of experiments (Study #01), the effect of giving compound 18 h prior to infection, versus 18 h post-infection was studied. The supernatant removed from the cytotoxicity plates at each medium change was stored at −20° C. for shipment to the client. The supernatant was collected at 10 days post infection and stored at −20° C. for total extracellular HBV DNA copy number to be quantitatively determined by qPCR. Cell viability was measured by XTT tetrazolium dye at day 10 for the cytotoxicity plates.

### Quantitative PCR Detection of Total HBV DNA in the Supernatant

Ten microliters (10 μL) of cell culture supernatant from the collections on day 10 were diluted into 90 μL buffer consisting of 10 mM Tris, 40 μg/mL sheared salmon sperm DNA and boiled for 15 minutes. Quantitative PCR (qPCR) was performed in 384 well plates using a Bio-Rad CFX384 Touch Realtime-PCR Detection System and the supporting CFX Manager software. Five microliters (5 μL) of diluted and boiled cell culture supernatant for each sample and serial 10-fold dilutions of a quantitative DNA standard were subjected to real time qPCR using Platinum Quantitative PCR SuperMix-UDG (lnvitrogen) and specific DNA oligonucleotide primers (IDT, Coralville, Id.) HBV-AD38-qF1 (5′-CCG TCT GTG CCT TCT CAT CTG-3′), HBV-AD38-qR1 (5′- AGT CCA AGA GTY CTC TTA TRY AAG ACC TT-3′), and HBV-AD38-qP1 (5′-FAM-CCG TGT GCA/ZEN/CTT CGC TTC ACC TCT GC-3′BHQ1) final concentration of 0.2 μmol/L for each primer in a total reaction volume of 25 μL. The quantity of HBV DNA in each sample was interpolated from the standard curve and the data were imported into an Excel spreadsheet for analysis.

### Cell Toxicity

Following incubation at 37° C. in a 5% CO₂ incubator for ten days, the test plates were stained with the tetrazolium dye XTT (2,3-bis(2-methoxy-4-nitro-5-sulfophenyl}-5-[(phenylamino)carbonyl]-2H-tetrazolium hydroxide). XTT-tetrazolium was metabolized by the mitochondrial enzymes of metabolically active cells to a soluble formazan product. XTT solution was prepared daily as a stock of 1 mg/mL in RPMI1640. Phenazine methosulfate (PMS) solution was prepared at 0.15 mg/mL in PBS and stored in the dark at −20° C.

XTT/PMS stock was prepared immediately before use by adding 40 μL of PMS per mL of XTT solution. Fifty microliters of XTT/PMS were added to each well of the plate and the plate was re-incubated for 4 hours at 37° C. Plates were sealed with adhesive plate sealers and shaken gently or inverted several times to mix the soluble formazan product and the plate was read spectrophotometrically at 450/650 nm with a Molecular Devices Vmax plate reader.

### Experimental Designs

The experiments were designed as shown in Table 3.

TABLE 3

| | | Concentrations |
|---|---|---|
| Study # | Test Item | (nmol/L) |
| 01 | Tenofovir disoproxil fumarate | 0.32-1000 |
| | Compound 1 | 0.01-1000 |
| | myrcludex B | 0.01-1000 |
| 02 | Tenofovir disoproxil fumarate | 6.25-100 |
| | Compound 1 | 6.25-200 |
| | Tenofovir disoproxil fumarate + | All combinations of |
| | Compound 1 | above concentrations |
| 03 | Tenofovir disoproxil fumarate | 6.25-100 |
| | Compound 1 | 1.6-50 |
| | Tenofovir disoproxil fumarate + | All combinations of |
| | Compound 1 | above concentrations |

### Data Analysis

Each concentration of inhibitor and controls were analyzed in triplicate for HBV DNA copies and for cellular toxicity, respectively. From control wells without inhibitor, the mean of these three replicates was calculated and the HBV DNA mean was set to 100% viral control and the XTT mean was set to 100% cell viability. The effect of compound was then expressed as % of viral control and % cell viability, respectively.

In two sets of experiments, the effect of combining Compound 1 with tenofovir was studied. Effects of the drug combination were calculated based on the activity of the two compounds when tested alone. The expected additive antiviral protection (based on the dose response curves of the individual agents used alone) was subtracted from the experimentally determined antiviral activity at each combination concentration resulting in a positive value (synergy), a negative value (antagonism), or zero (additivity). Data were analyzed by the most stringent statistical means by assuming the compounds inhibited HBV replication by action at the same site (mutually exclusive). The results of the combination assays were expressed as a synergy volume μM2% calculated at the 95% confidence interval using the MacSynergy II template.

For these studies, synergy was defined as drug combinations yielding synergy volumes greater than 50 μM2%. Slightly synergistic activity and highly synergistic activity have been defined as yielding synergy volumes of 50 to 10 μM2% and >100 μM2%, respectively.

Synergy volumes between −50 and 50 μM2% were considered additive and synergy volumes less than −50 μM2% were considered antagonistic.

### Results

### Study #01

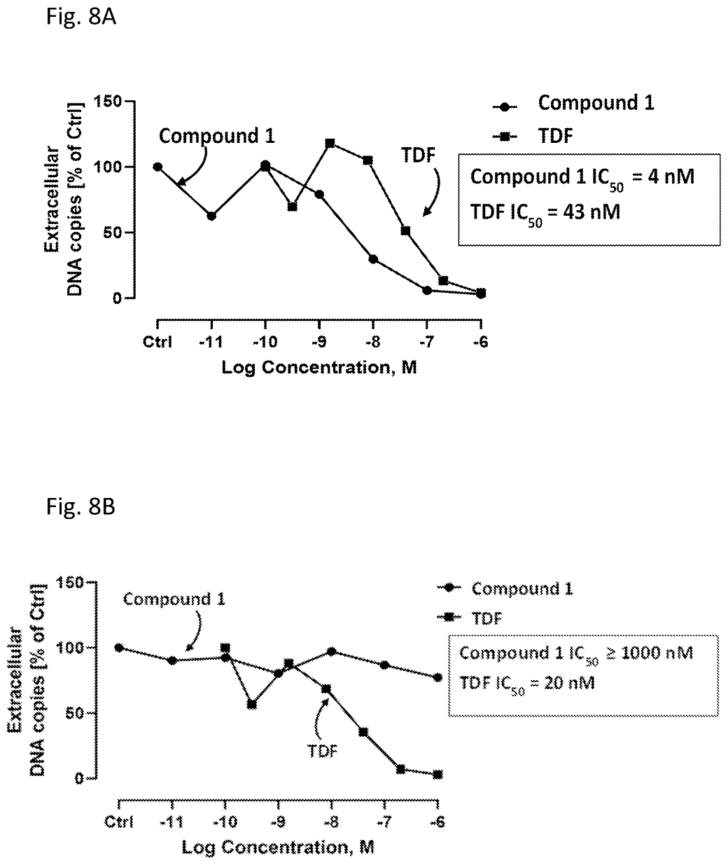

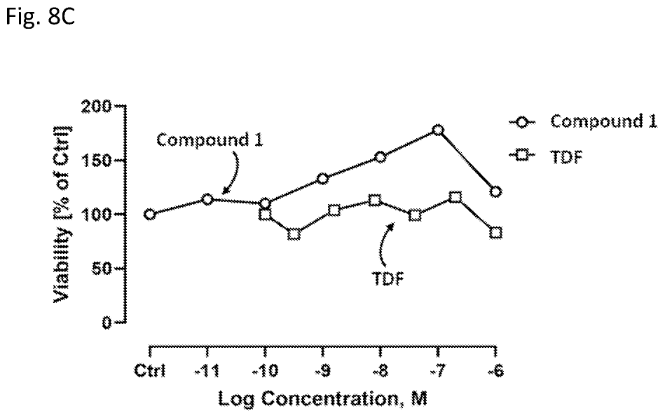

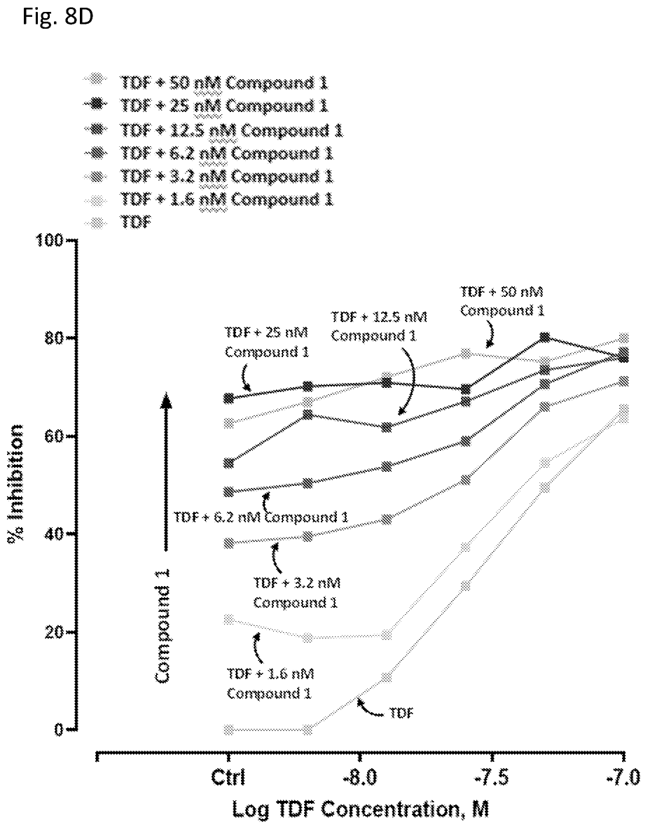

The EC₅₀ values for tenofovir and Compound 1, when giving the compounds 18 h prior to infection (standard conditions), are summarized in Table 4 and illustrated in FIG. 8A. Tenofovir demonstrated expected potency consistent with historical values (EC₅₀: 43 nmol/L). myrcludex B (EC₅₀: 0.4 nmol/L) and Compound 1 (EC₅₀: 4 nmol/L) were more potent than tenofovir. None of the compounds induced cytotoxicity at any concentration tested (highest concentration: 1 μmol/L). See FIG. 8C.

The EC₅₀ values for tenofovir, Compound 1 and myrcludex B, when giving the compounds 18 h post infection, are summarized in Table 4 and illustrated in FIG. 8B. The potency of tenofovir was maintained when giving the compound 18 h post-dosing, consistent with inhibition of HBV replication. By contrast, antiviral activity was lost when Compound 1 and myrcludex B were given 18 h post-infection, consistent with an anti-entry mechanism.

### Study #02

The effect of combining Compound 1 with tenofovir was also studied. The EC₅₀ values for tenofovir and Compound 1 are summarized in Table 4. Tenofovir demonstrated expected potency consistent with historical values (EC₅₀: 64 nmol/L). Compound 1 was more potent than tenofovir and a precise EC₅₀ could not be determined since 58% inhibition was present at the lowest concentration tested (6.25 nmol/L). None of the compounds induced cytotoxicity at any concentration tested (highest concentration: 0.1-0.2 μmol/L). Combinations of the two agents provided very potent anti-viral effects. As Compound 1 was highly effective on its own, judging if the combined effects were additive or synergistic was challenging; an additional study was performed.

### Study #03

The effect of combining lower concentrations of Compound 1 than in Study #02 with tenofovir was studied. The EC₅₀ values for tenofovir and Compound 1 when given alone are summarized in Table 4 and illustrated in FIG. 8D. Tenofovir demonstrated expected potency consistent with historical values (EC₅₀: 59 nmol/L). However, in contrast to previous experiments, the maximal inhibitory effect of tenofovir was ˜60%. Compound 1 was more potent with an EC₅₀ of 11 nmol/L. The maximal inhibitory effect of Compound 1 was also ˜60%. When combined, the data suggested that Compound 1 and tenofovir exerted additive effects.

TABLE 4
Data summary.

| | | | Max | |
|---|---|---|---|---|
| Study | | EC₅₀ | inhibition | TC₅₀ |
| # | Test Item | (nmol/L) | (%) | (μmol/L) |
| 01 | Tenofovir disoproxil fumarate | 43 | 96 | >1 |
| | Compound 1 | 4 | 97 | >1 |
| | myrcludex B | 0.4 | 97 | >1 |
| | 18 h post-infection: | | | |
| | Tenofovir disoproxil fumarate | 20 | 97 | >1 |
| | Compound 1 | >1 | — | >1 |
| | myrcludex B | >1 | — | >1 |
| 02 | Tenofovir disoproxil fumarate | 64 | 68 | >0.1 |
| | Compound 1 | <6.3 | 88 | >0.2 |
| | Tenofovir disoproxil | Additive | | |
| | fumarate + Compound 1 | | | |
| 03 | Tenofovir disoproxil fumarate | 59 | 61 | >0.1 |
| | Compound 1 | 11 | 63 | >0.05 |
| | Tenofovir disoproxil | Additive | | |
| | fumarate + Compound 1 | | | |

Compound 1 consistently inhibited HBV infection of human primary hepatocytes in a concentration-dependent manner. Compound 1 did not affect cell viability at any concentration tested (up to 100 μmol/L). Compound 1 produced additive antiviral effects when given together with tenofovir disoproxil fumarate.

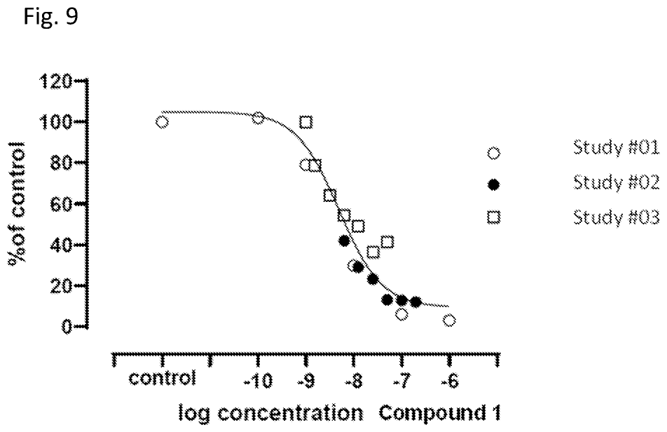

The antiviral effects of Compound 1 from Study #01, Study #02, and Study #03 are summarized in FIG. 9. The data fitted well to a non-linear, four parameter regression curve (R²: 0.93). From the fitted curve, an EC₅₀ of 4.9 nmol/L was determined.

### Example 5. Effect of Compound 1 in HBV-Infected PXB Mice

The plasma exposure after oral dosing of Compound 1 to humanized, non-infected PXB mice and the prophylactic anti-HBV effect of Compound 1 after oral dosing to PXB mice before HBV infection were evaluated.

### Methods

### Study Design of Pharmacokinetic Phase

The study was performed with Compound 1 with the experimental design as shown in Table 5.

TABLE 5

| Group | | Dose | Volume | | Number of |
|---|---|---|---|---|---|
| No. | Frequency | (mg/kg) | (mL/kg) | Route | Animals |
| 1 | Single | 10 | 10 | p.o. | 3 |
| | | | | | (101-103) |
| 2 | Single | 10 | 10 | p.o. | 3 |
| | | | | | (201-203) |
| 3 | Single | 30 | 10 | p.o. | 3 |
| | | | | | (301-303) |
| 4 | Single | 30 | 10 | p.o. | 3 |
| | | | | | (401-403) |

### Study Design of Main study

The study was performed according to experimental design shown in Table 6.

TABLE 6

| Group | | | Dose | Volume | | Number of |
|---|---|---|---|---|---|---|
| No. | Test Item | Frequency | (mg/kg) | (mL/kg) | Route | Animals/ID |
| 1 | Vehicle | Once daily | 0 | 10 | p.o. | 4 |
| | | for 15 days | | | | (101-104) |
| | | (Day 0*-14) | | | | |
| 2 | Compound 1 | Once daily | 10 | 10 | p.o. | 4 |
| | | for 15 days | | | | (201-204) |
| | | (Day 0*-14) | | | | |
| 3 | Compound 1 | Once daily | 30 | 10 | p.o. | 4 |
| | | for 15 days | | | | (301-304) |
| | | (Day 0*-14) | | | | |

*Dosing on Day 0 took place 60 minutes prior to HBV inoculation (0.1 mL HBV C inoculum (108 copies/mL) i.v.).

### Blood Sampling Pharmacokinetic Phase

Thirty microliters (30 μL) of blood were collected from all animals under isoflurane anesthesia via the retro-orbital plexus/sinus using sodium heparin-coated calibrated pipettes according to the collection schedule shown in Table 7.

TABLE 7

| | Dose | Timepoint post-dose |
|---|---|---|
| Group No. | (mg/kg) | (h) |
| 1 (n = 3) | 10 | 0.5, 2, 6 |
| 2 (n = 3) | 10 | 1, 4, 24 |
| 3 (n = 3) | 30 | 0.5, 2, 6 |
| 4 (n = 3) | 30 | 1, 4, 24 |

The individual blood samples of the animals were transferred to labeled microtubes and stored under chilled conditions until centrifugation at 3500×g, 5° C. for 10 minutes to obtain plasma. Individual plasma samples were transferred into separately labeled microtubes and snap frozen in liquid nitrogen.

### Blood Sampling Main Study

Target volume of blood was collected from all surviving animals under isoflurane anesthesia via the retro-orbital plexus/sinus using Intramedic™ Polyethylene Tubing at each time point. Two microliters (2 μL) from the collected blood were used for human albumin measurements. The remaining blood was centrifuged to separate serum to be used for analyzing the indicated biomarkers. Blood samples were collected according to Table 8.

TABLE 8

| | | Blood | Blood | | | |
|---|---|---|---|---|---|---|
| | | sample for | sample for | | | |
| | | human | serum | | | |
| | | albumin | biomarkers | HBV | | |
| Day | Timepoint | (μL) | (μL) | DNA | HBsAg | HBeAg |
| 0 | Pre-dose | 2 | 75 | X | X | X |
| 7 | Pre-dose | 2 | 50 | X | X | |
| 14 | Pre-dose | 2 | 75 | X | X | X |
| 21 | | 2 | 50 | X | X | |
| 28 | | 2 | 75 | X | X | X |
| 35 | | 2 | 50 | X | X | |
| 42 | | 2 | >300* | X | X | X |

*terminal collection

The individual blood samples of the animals were left at room temperature for at least 5 minutes to coagulate and then centrifuged at 13200×g, 4° C. for 3 minutes to obtain serum. The serum was divided into separate labeled microtubes and stored at −80° C. until analysis.

### Necropsy and Tissue Sampling

Whole livers from all animals were harvested and weighed at necropsy. Liver samples from the left lateral were immersed in RNAlater solution®, incubated overnight and stored at −80° C. until use.

### Bioanalysis

The plasma exposure of Compound 1 from the pharmacokinetic phase was measured via LC/MS- MS. The lower limit of quantitation was 1 ng/mL.

### Blood Human Albumin Concentration

The clinical chemistry analyzer (BioMajesty™ Series JCA-BM6050, JEOL Ltd., Tokyo, Japan) was used to measure the blood human albumin concentration using latex agglutination immunonephelometry.

### Serum HBV DNA

Real-time PCR detection was used to measure the serum HBV DNA concentration using the KUBIX HBV qPCR Kit (KUBIX Inc.) and CFX96 Touch™ Real-Time PCR Detection System (Bio-Rad Laboratories, Inc., Hercules, Calif., USA). Ten microliters (10 μL) of the HBV 2×PCR Solution were added into 10 μL of the heated sample. The initial activation was conducted at 95° C. for 2 minutes. Subsequent PCR amplification consisted of 45 cycles of denaturation at 95° C. for 5 seconds and annealing and extension at 54° C. for 30 seconds per cycle in a CFX96 Touch™ Real-Time PCR Detection System. The average serum HBV DNA level was calculated from the values of the two separate wells. The primers and probes are shown in Table 9.

TABLE 9

| Identi- | Target | Sequence Information | | |
|---|---|---|---|---|
| fication | Location | Dye | 5′ Nucleotides 3′ | Dye |
| Forward | 166-186 | n/a | CACATCAGGATTCCTAG | n/a |
| primer | | | GACC | |
| Reverse | 344-325 | n/a | AGGTTGGTGAGTGATTG | n/a |
| primer | | | GAG | |
| TaqMan | 242-267 | 6- | CAGAGTCTAGACTCGTG | TAMRA |
| probe | | FAM | GTGGACTTC | |

The lowest quantification limit of this assay was 4×10⁴ copies/mL serum.

### Serum HBsAg

Serum HBsAg concentration was determined by SRL, Inc. (Tokyo, Japan) based on a Chemiluminescent Enzyme Immuno Assay (CLEIA) developed by Fujirebio (LUMIPULSE HBsAg-HQ, LUMIPULSE® Prestoll). The dilution factor was 30 and 10, and the measurement range of this assay was between 0.005 and 150 IU/mL. For the 30-fold diluted samples, the measurement range was adjusted to be between 0.15 and 4500 IU/mL. And for the 10-fold diluted samples, the measurement range was adjusted to be between 0.05 and 1500 IU/mL. The lowest quantification limit of this assay was 0.15 IU/mL serum.

### Serum HBeAg

Serum HBeAg concentration was determined by SRL, Inc. based on a Chemiluminescent Enzyme Immuno Assay (CLEIA) developed by Fujirebio (LUMIPULSE HBeAg, LUMIPULSE® Prestoll). The dilution factor was 30 and 10, and the measurement range of this assay was between 0.1 and 1590 C.O.I. For the 30-fold diluted samples, the measurement range was adjusted to be between 3 and 47700 C.O.I. And for the 10-fold diluted samples, the measurement range was adjusted to be between 1 and 15900 C.O.I. The lowest quantification limit of this assay was 3.0 C.O.I.

### Liver HBV DNA

HBV DNA was extracted from frozen RNALATER®-preserved liver tissue using the DNEASY® Blood & Tissue Kits (Qiagen K.K., Tokyo, Japan). Hepatic HBV DNA concentration (expressed as copies/100 ng DNA) was determined using the TaqMan Fast Advanced Master Mix and CFX96 Touch™ Real-Time PCR Detection System (Bio-Rad Laboratories, Inc., Hercules, Calif., USA). The DNA was dissolved in 200 μL nuclease-free water, after which the concentration of the DNA solution was determined using SPECTRAMAX® ABS Plus Microplate Readers. The concentration of DNA solution was adjusted to 20 ng/μL using Nuclease-free water.

The standard for the hepatic HBV DNA quantification was extracted from 5 μL of serum using the SMITEST EX-R&D Nucleic Acid Extraction Kit (MEDICAL & BIOLOGICAL LABORATORIES CO., LTD., Nagoya, Japan). The DNA will be dissolved in 20 μL nuclease-free water.

Hepatic HBV DNA concentration (expressed as copies/100 ng DNA) was determined using the TaqMan Fast Advanced Master Mix and CFX96 Touch™ Real-Time PCR Detection System (Bio-Rad Laboratories, Inc., Hercules, Calif., USA). The PCR reaction mixture will be added into 5 μL of the extracted DNA. The PCR reaction mixture was added into 5 μL of the extracted DNA. The initial activation of uracil-N-glycosylase at 50° C. for 2 minutes was followed by the polymerase activation at 95° C. for 20 seconds. Subsequent PCR amplification consisted of 53 cycles of denaturation at 95° C. for 3 seconds and annealing and extension at 60° C. for 32 seconds per cycle in an CFX96 Touch™ Real-Time PCR Detection System. The average hepatitis HBV DNA level was calculated from the values of the two separate wells.

The lowest quantification limit of this assay was 50 copies/100 ng DNA in extracted DNA solution.

### Liver cccDNA

Real-time detection PCR was used to measure liver HBV cccDNA concentration using the TaqMan Fast Advanced Master Mix and CFX96 Touch™ Real-Time PCR Detection System (Bio-Rad Laboratories, Inc., Hercules, Calif., USA). The PCR reaction mixture was added into 5 μL of the extracted DNA. The PCR reaction was conducted based on the Takkenberg's condition). The initial activation of uracil-N-glycosylase at 50° C. for 2 minutes was followed by the polymerase activation at 95° C. for 20 seconds. Subsequent 55 cycles of PCR amplification were conducted at 95° C. for 3 seconds and annealing and extension at 60° C. for 32 seconds per cycle in a CFX96 Touch™ Real-Time PCR Detection System. The average HBV cccDNA level was calculated from the values of the two separate wells. The primers and probes are shown in Table 10.

TABLE 10

| Identi- | Target | Sequence Information | | |
|---|---|---|---|---|
| fication | Location | Dye | 5′ Nucleotides 3′ | Dye |
| Forward | 1545-1563 | n/a | CTCCCCGTCTGTGCCTT | n/a |
| primer | | | CT | |
| Reverse | 1900-1883 | n/a | GCCCCAAAGCCACCCAA | n/a |
| primer | | | G | |
| TaqMan | 1602-1628 | 6- | CGTCGCATGGARACCAC | TAMRA |
| probe | | FAM | CGTGAACGCC | |

The lowest quantification limit of this assay was 100 copies/100 ng DNA in extracted DNA solution.

### Results

### Exposure of Compound 1 after Oral Dosing to Non-Infected PXB Mice

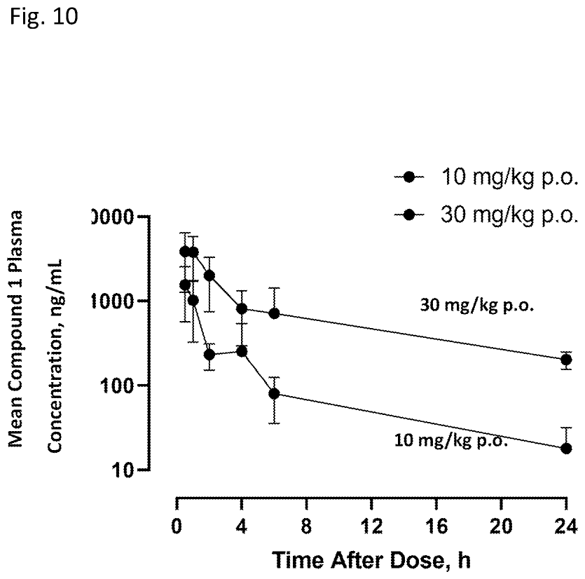

Table 11 summarizes the data from mice exposed to 10 mg/kg p.o. while Table 12 summarizes the data from mice exposed to 30 mg/kg p. o. The plasma vs time profiles are also depicted in FIG. 10. Compound 1 was detected in plasma throughout the 24-hour time period in both groups. Plasma exposure of Compound 1 increased with increased dose.

TABLE 11
Individual and mean Exposures of Compound 1 in non-infected PXB
mice after dosing 10 mg/kg (p.o.) in the pharmacokinetic phase

| Time | | | | | | | |
|---|---|---|---|---|---|---|---|
| (h) | Plasma conc. (ng/mL) | | | | | | Mean ± SD |
| Animal | 101 | 102 | 103 | 201 | 202 | 203 | (ng/mL) |
| 0.5 | 1290 | 2650 | 727 | — | — | — | 1560 ± 990 |
| 1 | — | — | — | 492 | 763 | 1810 | 1020 ± 690 |
| 2 | 194 | 322 | 177 | — | — | — | 231 ± 79 |
| 4 | — | — | — | 111 | 61.4 | 587 | 253 ± 290 |
| 6 | 46.2 | 63.5 | 130 | — | — | — | 80.0 ± 44 |
| 24 | — | — | — | 25.3 | 27.2 | 1.40 | 18.0 ± 14 |

TABLE 12
Individual and mean Exposure of Compound 1 in non-infected PXB
mice after dosing 30 mg/kg (p.o.) in the pharmacokinetic phase

| Time | | | | | | | |
|---|---|---|---|---|---|---|---|
| (h) | Plasma conc. (ng/mL) | | | | | | Mean ± SD |
| Animal | 301 | 302 | 303 | 401 | 402 | 403 | (ng/mL) |
| 0.5 | 1880 | 2910 | 6810 | — | — | — | 3870 ± 2600 |
| 1 | — | — | — | 4560 | 5320 | 1470 | 3780 ± 2000 |
| 2 | 2420 | 590 | 3040 | — | — | — | 2020 ± 1300 |
| 4 | — | — | — | 1120 | 1090 | 217 | 811 ± 510 |
| 6 | 554 | 99.8 | 1490 | — | — | — | 715 ± 700 |
| 24 | — | — | — | 230 | 229 | 148 | 202 ± 47 |

### Effect of Compound 1 in HBV-Infected PXB Mice

On Day 16, animal number 102 and on Day 23, animal number 104 were found dead. Both were from the vehicle-treated group. Cause of death was believed to be intestinal hemorrhage. On Day 33, animal 203 from the 10 mg/kg Compound 1 group was found dead. Cause of death was believed to be metastatic thymoma, which has been reported as being a spontaneous change in SCID mice. No noteworthy observations related to Compound 1 were found.

### Body Weight

Body weight remained constant throughout the study. No differences were found between the groups.

### Human Albumin Blood Concentrations

During the treatment period, the blood concentration of human albumin was stable in the Compound 1-treated animals (10 mg/kg Compound 1, baseline: 10.8±1.0 mg/mL, Day 14: 10.8±0.9 mg/mL; 30 mg/kg Compound 1, baseline: 10.6±1.5 mg/mL, Day 14: 11.3±1.2 mg/mL) while tended to decrease in the vehicle-treated group (baseline: 10.7±0.9 mg/mL, Day 14: 8.1±2.6 mg/mL). The decrease in the vehicle group was mainly due to the two animals subsequently found dead on Days 16 and 23.

### Serum HBV DNA

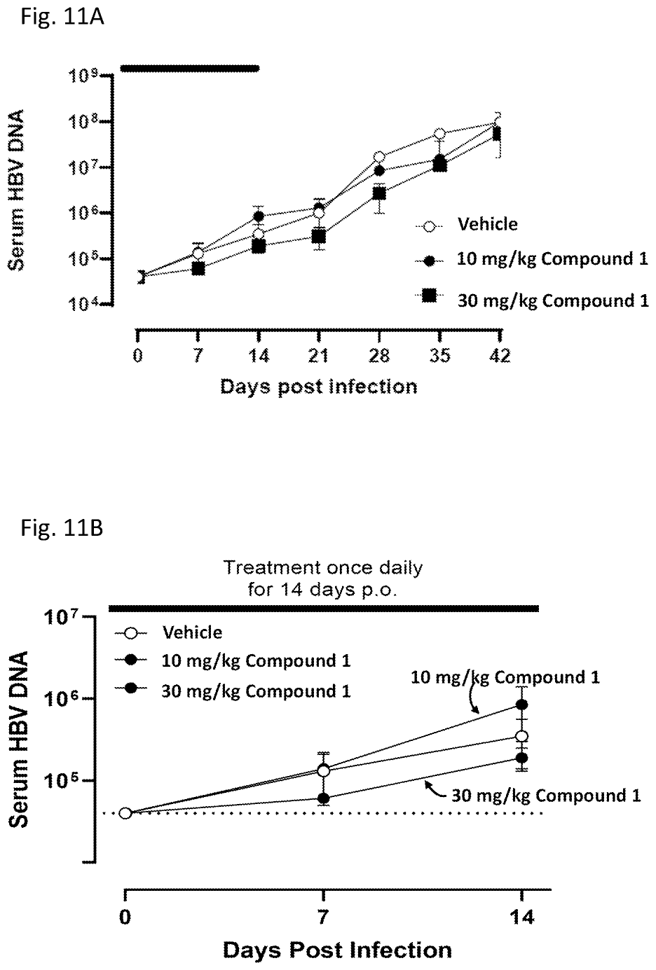

A summary of HBV DNA concentrations is illustrated in FIGS. 11A and 11B. HBV DNA was detected on Day 7 and was lower in mice treated with 30 mg/kg Compound 1 compared to controls. The lower HBV DNA levels in response to Compound 1 were maintained throughout the study until planned termination on Day 42 despite treatment being stopped on Day 14 as planned.

### Serum HBsAg

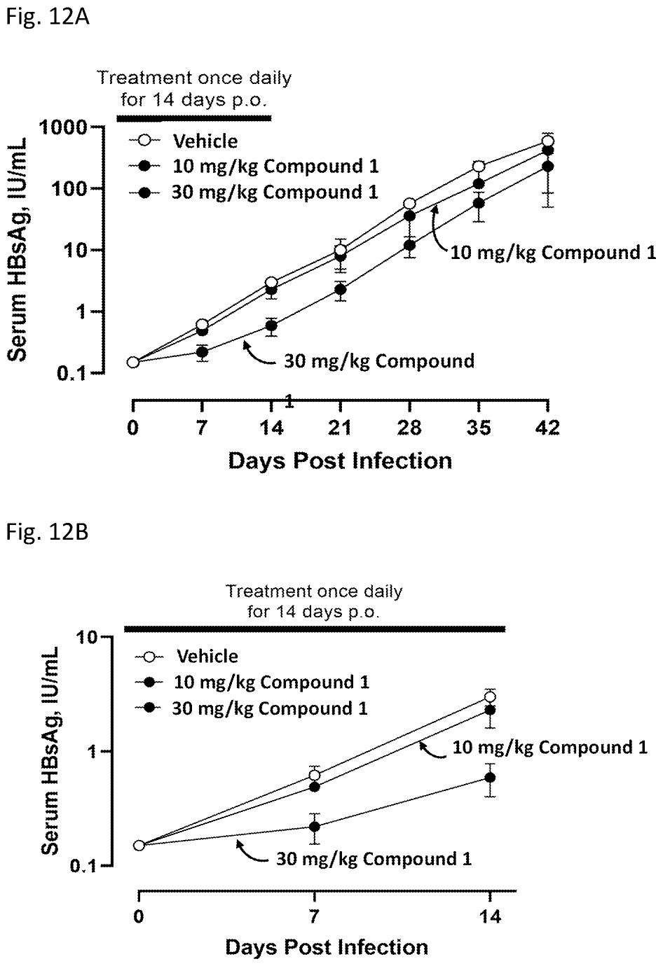

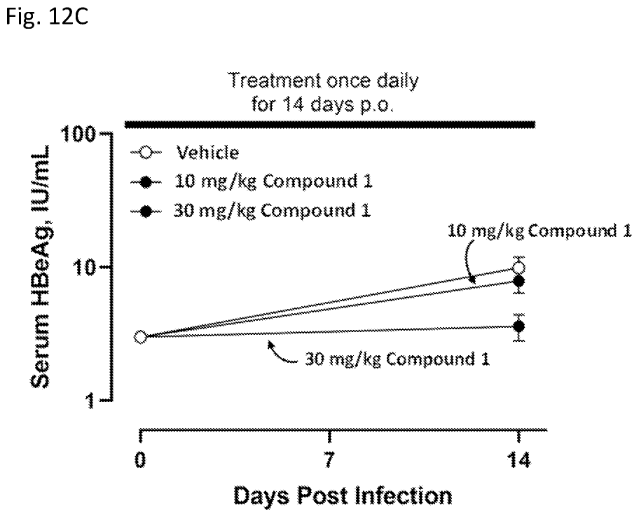

A summary of HBsAg concentrations is illustrated in FIGS. 12A and 12B. HBsAg was detected on Day 7 and was lower in mice treated with 10 mg/kg and 30 mg/kg Compound 1 compared to controls. The lower HBsAg levels in response to both doses of Compound 1 were maintained throughout the study until planned termination on Day 42 despite treatment being stopped on Day 14 as planned.

### Serum HBeAg

A summary of HBeAg concentrations is illustrated in FIG. 12C. HBeAg was detected on Day 14 (first timepoint measured) and was lower in mice treated with 10 mg/kg and 30 mg/kg Compound 1 compared to controls. The lower HBeAg levels in response to both doses of Compound 1 were maintained throughout the study until planned termination on Day 42 despite treatment being stopped on Day 14 as planned.

### Hepatic HBV DNA and HBV cccDNA Levels

A summary of hepatic HBV DNA and HBV cccDNA levels at planned termination on Day 42 can be found in Table 13. There were no differences between the treatment groups.

TABLE 13
Effect of Compound 1 on serum HBV DNA levels.

| | Day | | | | | | |
|---|---|---|---|---|---|---|---|
| Treatment | 0 | 7 | 14 | 21 | 28 | 35 | 42 |
| Vehicle | 4.0 × 10⁴ | 1.3 × 10⁵ | 3.5 × 10⁵ | 1.0 × 10⁶* | 1.7 × 10⁷** | 5.5 × 10⁷** | 9.7 × 10⁷** |
| | (0) | (7.1 × 10⁴) | (3.5 × 10⁵) | (1.2 × 10⁶) | — | — | — |
| Compound 1 | 4.0 × 10⁴ | 1.4 × 10⁵ | 8.5 × 10⁵ | 1.3 × 10⁶ | 8.5 × 10⁶ | 2.7 × 10⁷* | 1.2 × 10⁸* |
| 10 mg/kg | (0) | (9.2 × 10⁴) | (8.8 × 10⁵) | (1.8 × 10⁶) | (2.0 × 10⁷) | (6.2 × 10⁷) | (1.9 × 10⁸) |
| Compound 1 | 4.0 × 10⁴ | 6.1 × 10⁴ | 1.9 × 10⁵ | 3.1 × 10⁵ | 2.7 × 10⁶ | 1.1 × 10⁷ | 5.4 × 10⁷ |
| 30 mg/kg | (0) | (4.2 × 10⁴) | (1.2 × 10⁵) | (3.0 × 10⁵) | (3.4 × 10⁶) | (1.2 × 10⁷) | (7.7 × 10⁷) |

### Discussion

In non-infected PXB mice, Compound 1 was detected in plasma throughout the 24-hour time period when given at 10 and 30 mg/kg (p.o.). Plasma exposure of Compound 1 increased with increased dose. Compound 1 had prophylactic effects against HBV genotype C infection in PXB-mice. Serum levels of HBsAg (FIGS. 12A and 12B), HBeAg (FIG. 12C), and HBV DNA (FIGS. 11A and 11B) were lower in the mice treated with 30 mg/kg Compound 1 (n=4) vs 10 mg/kg Compound 1 (n=4) and vehicle-treated mice (n=2), especially during the 14-day treatment period. After treatment, markers of HBV infection remained consistently lower in the 30 mg/kg Compound 1-treated mice vs the 10 mg/kg Compound 1 and vehicle control groups.

### Example 6. Characterization of Compound 2

Na+-taurocholate co-transporting polypeptide (NTCP) is a hepatocyte sinusoidal membrane bile acid (BA) transporter. In addition to its major role in hepatocellular clearance of BAs, NTCP is used by hepatitis B and D viruses (HBV, HDV) to enter and infect human hepatocytes. The effects of Compound 2 on the inhibition of BA uptake and HBV entry were evaluated.

### Methods

NTCP and the related BA transporter apical sodium-dependent BA transporter (ASBT) from human were expressed in cell lines. Inhibition by Compound 2 was studied using [³]taurocholate as substrate. Cryopreserved human hepatocytes infected with a clinical isolate of HBV were used for infection experiments. Two female non-human primates (NHPs) were dosed with Compound 2 via oral gavage at dose levels of Compound 2 ranging between 3-30 mg/kg once daily for 5 days. Total serum, urine, and fecal BAs were analyzed enzymatically and Compound 2 plasma concentrations were assessed via liquid chromatography-tandem mass spectrometry. The oral pharmacokinetics of Compound 2 was also studied in mice, rats, and dogs.

### Results

Compound 2 had half-maximal inhibitory concentration (IC₅₀) values of 3.4 and 193 nmol/L vs human NTCP and ASBT, respectively. Compound 2 prevented HBV infection of human hepatocytes with an IC₅₀ of 13.5 nmol/L without affecting cell viability. In NHPs, plasma exposure of Compound 2 increased with dose, and serum BAs increased in a dose-dependent manner in response to oral dosing of Compound 2 on both Day 1 and Day 5. Doses of 10 and 30 mg/kg Compound 2 evoked increases in serum BAs lasting for at least 8 hours on both Day 1 and Day 5. Serum BAs returned to baseline at 24 hours post-dose in response to 10 mg/kg A7387 while still being elevated in response to 30 mg/kg Compound 2. Urine excretion of BAs tended to increase in response to Compound 2 compared with vehicle, while the levels of fecal BAs did not change. Levels of the BA synthesis biomarker 7-alpha-hydroxy-4-cholesten-3-one (C4) did not change in response to Compound 2 after 5 days of repeated dosing. Good oral bioavailability was demonstrated in the pharmacokinetic studies across all species. Compound 2 is a highly potent, selective, orally available NTCP inhibitor with potential in HBV/HDV infection and cholestatic diseases.

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

1. A method for treating hepatitis B (HBV) in a subject in need thereof, the method comprising orally administering to the subject a therapeutically effective amount of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

2. A method for preventing or decreasing entry of a hepatitis B viral particle into a hepatocyte in a subject in need thereof, the method comprising administering to the subject a therapeutically effective amount of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

3. A method for decreasing hepatitis B viral replication in a hepatocyte in a subject in need thereof, the method comprising administering to the subject a therapeutically effective amount of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

4. The method of claim 1, wherein the subject has hepatitis D.

5. A method for treating hepatitis D (HDV) in a subject in need thereof, the method comprising orally administering to the subject a therapeutically effective amount of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

6. A method for preventing or decreasing entry of a hepatitis D viral particle into a hepatocyte in a subject in need thereof, the method comprising administering to the subject a therapeutically effective amount of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

7. A method for decreasing hepatitis D viral replication in a hepatocyte in a subject in need thereof, the method comprising administering to the subject a therapeutically effective amount of (Z)-3- ((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

8. The method of claim 1, wherein the method further comprises administering an additional anti-viral agent.

9. A method for treating HBV in a subject in need thereof, the method comprising orally administering to the subject a therapeutically effective amount of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, and an additional anti-viral agent.

10. A method for treating HDV in a subject in need thereof, the method comprising orally administering to the subject a therapeutically effective amount of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, and an additional anti-viral agent.

11. The method of claim 8, wherein the additional anti-viral agent is selected from the group consisting of: entecavir, tenofovir, tenofovir disoproxil, tenofovir alafenamide, lamivudine, adefovir, adefovir dipivoxil, telbivudine, bulevirtide, an interferon, and a combination thereof.

12. The method of claim 11, wherein the interferon is pegylated interferon, interferon alpha, or a combination thereof.

13. The method of claim 8, wherein the additional anti-viral agent is tenofovir disoproxil.

14. The method of claim 5, wherein the subject has hepatitis B.

15. The method of claim 5, wherein the subject has chronic hepatitis B.

16. The method of claim 5, wherein the subject has chronic hepatitis D.

17. The method of claim 1, wherein the concentration of one or more biomarkers selected from HBV DNA, hepatitis B surface antigen (HBsAg), hepatitis B core antigen (HBcAg), hepatitis B e antigen (HBeAg), HDV DNA, and hepatitis D antigen (HDAg) in the serum of the subject decreases after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

18. The method of claim 17, wherein the concentration of HBV DNA in the serum of the subject decreases after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

19. The method of claim 17, wherein the concentration of HBV DNA is determined in a serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

20. The method of claim 18, wherein the concentration of HBV DNA in the serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, is decreased as compared to a reference concentration of HBV DNA.

21. The method of claim 19, wherein the reference concentration of HBV DNA is a level of HBV DNA in a serum sample obtained from the subject prior to administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

22. The method of claim 17, wherein the concentration of HBV DNA in the serum of the subject is decreased by about 10% to about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

23. The method of claim 17, wherein the concentration of HBV DNA in the serum of the subject is decreased by about 5%, about 10%, about 25%, about 50%, about 75%, or about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

24. The method of claim 17, wherein the concentration of HBV DNA in the serum of the subject is undetectable after administration of (Z)-3-((3-butyl-2- methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

25. The method of claim 17, wherein the concentration of hepatitis B surface antigen (HBsAg) in the serum of the subject decreases after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

26. The method of claim 25, wherein the concentration of HBsAg is determined in a serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

27. The method of claim 26, wherein the concentration of HBsAg in the serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, is decreased as compared to a reference concentration of HBsAg.

28. The method of claim 27, wherein the reference concentration of HBsAg is a concentration of HBsAg in a serum sample obtained from the subject prior to administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

29. The method of claim 25, wherein the concentration of HBsAg in the serum of the subject is decreased by about 10% to about to about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

30. The method of claim 25, wherein the concentration of HBsAg in the serum of the subject is decreased by about 5%, about 10%, about 25%, about 50%, about 75%, or about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

31. The method of claim 25, wherein the concentration of HBsAg in the serum of the subject is undetectable after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

32. The method of claim 17, wherein the concentration of hepatitis B core antigen (HBcAg) in the serum of the subject decreases after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

33. The method of claim 32, wherein the concentration of HBcAg is determined in a sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

34. The method of claim 33, wherein the concentration of HBcAg in the serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, is decreased as compared to a reference concentration of HBcAg.

35. The method of claim 34, wherein the reference concentration of HBcAg is a concentration of HBcAg in a serum sample obtained from the subject prior to administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

36. The method of claim 32, wherein the concentration of HBcAg in the serum of the subject is decreased by about 10% to about to about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

37. The method of claim 32, wherein the concentration of HBcAg in the serum of the subject is decreased by about 5%, about 10%, about 25%, about 50%, about 75%, or about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

38. The method of claim 35, wherein the concentration of HBcAg in the serum of the subject is undetectable after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

39. The method of claim 17, wherein the concentration of hepatitis B e antigen (HBeAg) in the serum of the subject decreases after administration of (Z)-3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

40. The method of claim 39, wherein the concentration of HBeAg is determined in a serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

41. The method of claim 40, wherein the concentration of HBeAg in the serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, is decreased as compared to a reference concentration of HBeAg.

42. The method of claim 41, wherein the reference concentration of HBeAg is a concentration of HBeAg in a serum sample obtained from the subject prior to administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

43. The method of claim 39, wherein the concentration of HBeAg in the serum of the subject is decreased by about 10% to about to about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

44. The method of claim 39, wherein the concentration of HBeAg in the serum of the subject is decreased by about 5%, about 10%, about 25%, about 50%, about 75%, or about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

45. The method of claim 39, wherein the concentration of HBeAg in the serum of the subject is undetectable after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

46. The method of claim 17, wherein the concentration of HDV DNA in the serum of the subject decreases after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

47. The method of claim 46, wherein the concentration of HDV DNA is determined in a serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

48. The method of claim 47, wherein the concentration of HDV DNA in the serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, is decreased as compared to a reference concentration of HDV DNA.

49. The method of claim 48, wherein the reference concentration of HDV DNA is a concentration of HDV DNA in a serum sample obtained from the subject prior to administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

50. The method of claim 46, wherein the concentration of HDV DNA in the serum of the subject is decreased by about 10% to about to about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2- fluoroacrylic acid, or a pharmaceutically acceptable salt thereof.

51. The method of claim 46, wherein the concentration of HDV DNA in the serum of the subject is decreased by about 5%, about 10%, about 25%, about 50%, about 75%, or about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

52. The method of claim 46, wherein the concentration of HDV DNA in the serum of the subject is undetectable after administration of (Z)-3-((3-butyl-2- methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

53. The method of claim 17, wherein the concentration of hepatitis D antigen (HDAg) in the serum of the subject decreases after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

54. The method of claim 53, wherein the concentration of HDAg is determined in a serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

55. The method of claim 54, wherein the concentration of HDAg in the serum sample from the subject obtained after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, is decreased as compared to a reference concentration of HDAg.

56. The method of claim 55, wherein the reference concentration of HDAg is a concentration of HDAg in a serum sample obtained from the subject prior to administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

57. The method of claim 53, wherein the concentration of HDAg in the serum of the subject is decreased by about 10% to about to about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

58. The method of claim 53, wherein the concentration of HDAg in the serum of the subject is decreased by about 5%, about 10%, about 25%, about 50%, about 75%, or about 99% after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

59. The method of claim 53, wherein the concentration of HDAg in the serum of the subject is undetectable after administration of (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof.

60. The method of claim 1, wherein the subject is administered (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, within 18 hours of exposure to hepatitis B.

61. The method of claim 1, wherein the subject is administered (Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, within 18 hours of exposure to hepatitis D.

62. The method of claim 1, wherein a therapeutically effective amount of (R)-(Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, is administered to the subject.

63. The method of claim 1, wherein a therapeutically effective amount of (S)-(Z)-3-((3-butyl-2-methyl-7-(methylthio)-1,1-dioxido-5-phenyl-2,3,4,5-tetrahydro-1,2,5-benzothiadiazepin-8-yl)oxy)-2-fluoroacrylic acid, or a pharmaceutically acceptable salt thereof, is administered to the subject.
